(19) United States
(12) Patent Application Publication
(10) Pub. No.: US 2022/0144895 A1
(43) Pub. Date: May 12, 2022

(54) DE NOVO DESIGN OF TUNABLE PH-DRIVEN CONFORMATIONAL SWITCHES

(71) Applicant: University of Washington, Seattle, WA (US)

(72) Inventors: Scott BOYKEN, Seattle, WA (US); David BAKER, Seattle, WA (US); Zibo CHEN, Seattle, WA (US); Alfredo Quijano RUBIO, Seattle, WA (US); Neil P. KING, Seattle, WA (US); Jason C. KLIMA, Seattle, WA (US); Carl WALKEY, Seattle, WA (US)

(21) Appl. No.: 17/602,119

(22) PCT Filed: Apr. 20, 2020
(86) PCT No.: PCT/US2020/028928

§ 371 Date: Oct. 7, 2021

Related U.S. Application Data
(60) Provisional application No. 62/835,651, filed on Apr. 18, 2019.

(51) Int. Cl. C07K 7/08

(52) U.S. Cl. CPC C07K 7/08

(57) ABSTRACT

Disclosed herein are polypeptides or polypeptide oligomers, including a buried hydrogen bond network that includes at least (1, 2, 3, 4, 5, 6, 7, 8, or 9) pH sensitive amino acids located (i) at an intra-chain interface between different: structural elements in one polypeptide, or (it) at an inter-chain interface between structural elements present in different chains of a polypeptide oligomer, wherein the polypeptide or polypeptide oligomer is stable above a given pH, and wherein the polypeptide or polypeptide oligomer undergoes a conformational transition when subjected to a pH at or below the given pH.

### CROSS REFERENCE

This application claims priority to U.S. Provisional Application Ser. No. 62/835,651 filed Apr. 18, 2019, incorporated by reference herein in its entirety.

### REFERENCE TO SEQUENCE LISTING

This application contains a Sequence Listing submitted as an electronic text file named “18-1784-PCT_Sequence-isting_ST25.txt”, having a size in bytes of 205 kb, and created on Apr. 19, 2020. The information contained in this electronic file is hereby incorporated by reference in its entirety pursuant to 37 CFR § 1.52(e)(5).

### BACKGROUND

The ability of naturally occurring proteins to change conformation in response to environmental changes is critical to biological function. While there have been advances in the de novo design of extremely stable proteins, the design of conformational switches remains a major challenge.

### SUMMARY

In one aspect, the disclosure provides non-naturally occurring polypeptides or polypeptide oligomers, comprising a buried hydrogen bond network that comprises at least 1, 2, 3, 4, 5, 6, 7, 8, or 9 pH sensitive amino acids located (i) at an intra-chain interface between different structural elements in one polypeptide, or (ii) at an inter-chain interface between structural elements present in different chains of a polypeptide oligomer, wherein the polypeptide or polypeptide oligomer is stable above a given pH, and wherein the polypeptide or polypeptide oligomer undergoes a conformational transition when subjected to a pH at or below the given pH. In one embodiment, the pH sensitive amino acids are selected from the group consisting of histidine, aspartate, and glutamate residues. In another embodiment, the different structural elements are selected from the group consisting of loops, beta sheets, alpha helices, or combinations thereof. In another embodiment, the at least one pH sensitive amino acid located is at an intra-chain interface between different structural elements in the polypeptide. In a further embodiment, the at least one pH sensitive amino acid located is at an inter-chain interface between structural elements present in different chains of the polypeptide oligomer. In one embodiment, the pH sensitive amino acids comprise histidine residues.

In another embodiment, the disclosure provides non-naturally occurring pH-responsive polypeptides, comprising an oligomeric helical bundle comprising at least four alpha-helical subunits, wherein the oligomeric helical bundle comprises:

one or more interfaces; and

one or more histidine-containing layers that participate in buried hydrogen bond networks, wherein each histidine N_{ε} and N_{δ} atoms are hydrogen-bonded across the one or more interfaces;

wherein the polypeptide is stable above a given pH, and wherein oligomers (including but not limited to dimers or trimers) of the polypeptide undergo a conformational transition when subjected to a pH at or below the given pH.

In a further embodiment, the disclosure provides non-naturally occurring pH-responsive polypeptides or polypeptide oligomers, comprising a helical bundle comprising at least four alpha-helical subunits, wherein the helical bundle comprises:

one or more interfaces; and

one or more histidine-containing layers that participate in buried hydrogen bond networks, wherein each histidine N_{ε} and N_{δ} atoms are hydrogen-bonded across the one or more interfaces;

wherein the polypeptide or polypeptide oligomer is stable above a given pH, and wherein the polypeptide or polypeptide oligomer undergoes a conformational transition when subjected to a pH at or below the given pH.

In various embodiments, the polypeptides comprise a polypeptide of general formula 1, 2, 3, or 4, as disclosed herein. In one embodiment, the polypeptide or polypeptide oligomers of any embodiment or combination of embodiments further comprises a functional subunit. In some embodiments, the functional subunit comprises a detectable protein or functional fragment thereof, including but not limited to a fluorescent protein or functional fragment thereof. In another embodiment, the polypeptides of the disclosure comprise the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to the polypeptide of any one of SEQ ID NOS:1-40, 45-46, 60-66, 69-76, and 81-86.

In another aspect, the disclosure provides non-naturally occurring polypeptides, comprising the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to the amino acid sequence of one of SEQ ID NOS:1-77 and 81-86. In another embodiment, the disclosure provides oligomeric polypeptides comprising two or more polypeptides of ay embodiment or combination of embodiments disclosed herein. In one embodiment, the oligomeric polypeptides comprise hetero-oligomers, including but not limited to a heterodimer of two different polypeptides. In another embodiment, the oligomeric polypeptides comprise homo-oligomers, including but not limited to a homotrimer.

The disclosure further comprises nucleic acids encoding the polypeptide of any embodiment or combination of embodiments disclosed herein, recombinant expression vectors comprising the nucleic acids operatively linked to a control sequence, cells comprising the nucleic acid ad/or the recombinant expression vector of the disclosure, uses of the polypeptides or the oligomeric polypeptides for any methods as disclosed herein, and methods for designing the polypeptides or the oligomeric polypeptides disclosed herein.

### DESCRIPTION OF THE FIGURES

FIG. 1A-G. Design of pH-responsive oligomers (pRO's). Design models indicate cross-sections that contain the histidine hydrogen bond networks. (A) Design strategy: pre-organized histidine residues destabilize intermolecular interfaces upon protonation at low pH. (B) The histidine-containing hydrogen bond networks of design pRO-2 (top) are replaced in pRO-2-noHis with networks with no histidines, but all buried polar atoms satisfied by hydrogen bonds (blue box, bottom). (C) pRO-2(top), but not pRO-2-noHis (bottom) undergoes cooperative pH-dependent quaternary structure disassociation when the pH is dropped below 5.5. Native mass spectrometry was carried out at indicated pH values at 5 μM trimer. (D) The stability of pRO-2 (top) but not pRO-2-noHis (bottom) is strongly pH dependent, as indicated by chemical denaturation with GdmCl monitored by circular dichroism (CD) mean residue ellipticity (MRE) at 222 nm. (E) pRO-2 CD wavelength scan and temperature met monitoring 222 am (Inset) for pRO-2 in Na₂HPO₄-Citrate buffer pH 7.0 (black), PBS pH 7.4 (dark), and PBS pH 7.4 with 10 mM EDTA (light). (F) Designed homotrimer pRO-3 and heterodimers pRO-4 and pRO-S. (G) pH-induced disassembly of designs in (F) monitored by native mass spectrometry; L23A/V130A mutation designed to weaken the interface of pRO-4 increase pH-sensitivity (dashed lines) compared to the parent design (solid lines). In (C) and (G), % oligomer is plotted as the percentage of that species relative to all oligomeric species observed at each pH value; for clarity, not all species are shown, and in several cases, other oligomeric species were observed at intermediate pH values during the transition to monomer (FIG. 20).

FIG. 2A-B. High resolution X-ray crystal structures are very close to design models. (A) Design models of pRO-2.3 and pRO-2.5 are in close agreement with (B) X-ray crystal structures (white); electron density (mesh) shown at a level of 1.0 Å; RMSD values between crystal structure and design model are given for heavy-atom superposition of the side chains shown in the boxes, and for all backbone atoms (right). Cross-section (layer) labels m, n, and l correspond to Eq. 1 and FIG. 3. Protein Data Bank (PDB) accession codes are 6MSQ (pRO-2.3) and 6MSR (pRO-2.5).

FIG. 3A-E. High Systematic tuning of pH transition point and cooperativity. (A) Schematics of designs with different combinations of hydrophobic layers (n, black), histidine network layers (m), and polar network layers lacking histidine (l); the number of each type of layer is given in parenthesis as (n, m, l). (B) Chemical denaturation by guanidinium chloride (GdmCl) at pH 7.4 measured by circular dichroism (CD) mean residue ellipticity (MRE) monitoring helicity at 222 nm. (C) Theoretical pH-dependence of trimer abundance according to Eq. 1; each curve corresponds to the values of m, n, and l for a design in (A) and are colored accordingly. ΔG_{hydrophobic}, ΔG_{polar_m}, and ΔG_{polar_I} were estimated from chemical denaturation experiments (B and FIG. 11). (D) Native mass spectrometry monitoring pH-induced quaternary structure disruption of the designs in (A) at 1.67 μM or 5 μM with respect to the trimeric species; curves were fit to the experimental data using Eq. 2. (E) The higher the ratio of m to n (x-axis), the higher the pH transition point pH₀ (y-axis).

FIG. 4A-E. pH-dependent membrane disruption. Proteins were added to synthetic liposomes encapsulating quenched sulforhodamine B (SRB) fluorescent dye; activity is measured by normalized dequenching of dye that leaks out from disrupted membranes. (A) Design pRO-2 disrupts liposomes in a pH-dependent manner, colors correspond to different pH values (shown on right). (B) pRO-2-noHis, which is not pH-responsive (FIG. 1C-1D), shows no detectable liposome activity at pH 5. (C) Design pRO-3 shows liposome disruption activity at pH 4.75, whereas pRO-3.1 does not, despite pRO-3.1 being more pH-responsive (FIG. 3D). (D) Comparison between pRO-2, pRO-3, pRO-3.1 suggests that the membrane interacting region is the contiguous hydrophobic stretch at the termini. Top to bottom: SEQ ID NOS:78, 79, and 80. (E) pRO-2170N mutation attenuates liposome activity. All liposome experiments used a final protein concentration of 2.5 μM with respect to monomer. All data shown on same plot was collected using the same batch of liposomes.

FIG. 5A-G. Imaging of pH-induced membrane permeabilization. (A) Tuning ΔG_{hydrophobic} by mutagenesis to increase the pH-sensitivity of pRO-2; (left) theoretical curves (Eq. 1) for pRO-2 compared to I56V and A54M mutants; (right) native mass spectrometry of pRO-2 compared to I56V and A54M mutants. The pH set point is shifted as predicted without affecting cooperativity; data are fit to Eq. 2 as in FIG. 3. (B) pRO-2 I56V has increased membrane permeabilization activity (assay as in FIG. 4). (C) Cryo-electron microscopy using purified proteins conjugated to gold-nanoparticles: design pRO-2 I56V interacts directly with liposomes at pH 5 but not pH 8, whereas pRO-2-noHis does not interact with liposomes at either pH. At low pH, design pRO-2 I56V deforms liposomes and induces the formation of tight extended interfaces between liposomes (white arrow in top middle panel; density between membranes is likely pRO-2 I56V). In all control conditions, liposomes were unperturbed and free protein conjugated gold-nanoparticles were well dispersed. All scale bars are equal to 100 nm. (D) Electron tomography of +36GFP fusions to pRO-2 and pRO-2-noHis at pH 5 or g. (E) Fluorescence imaging of +36GFP fusions to designs pRO-2, pRO-2 I56V, and pRO-2-noHis and composite correlation with lysosome membrane staining in U2-OS cells. pRO-2 I56V but not pRO-2-noHis is clearly localized within lysosomes; the pRO-2-noHis staining is likely from protease resistant aggregates. (F) Manders' colocalization coefficients representing the fraction +360FP fusion proteins colocalizing with lyscsomal membrane. (G) Ratios of yellow emission and blue emission on U2-OS loaded with LysoSensor™ Yellow/Blue DND-160 after 1 br incubation of pRO-2 (5 μM), pRO-2 I56V (3 μM), pRO-2-noHis (5 μM), Bafilomycin A (1 μM, Baf A), Chloroquine (50 μM), and medium (normal). The lower the ratio, the higher the lysosome pH; pRO-2 I56V increases the lysosomal pH more than the small molecule drugs.

FIG. 6A-B. (A) Homotrimer design pRO-1 was shown to be primarily dimeric at 7.5 μM dimer concentration by (B) native mass spectrometry. The mass spectrum was acquired on an Exactive Plus EMR Orbitrap™ mass spectrometer (Thermo Scientific) modified with a quadrupole mass filter and an SID device (56). Unlike successful designs pRO-2 to 5, which have contiguous, extensive histidine networks at each cross section. pRO-1 consists of three separate disjoint networks at each cross section, each with only a single histidine.

FIG. 7A-B. Designed homotrimer 2L6C3_13 has no histidine networks and is not pH-sensitive. (A) Native mass spectrometry was carried out at indicated pH values at 5 μM trimer concentration as in FIG. 1. (B) GdmCl denaturation experiment by CD monitoring the helical signal at 222 nM; compared to phosphate buffered saline (PBS) at pH 7.4 (gray), the same experiment in Na₂PO₄-Citrate at lower pH showed no destabilization, and in fact, lower pH seems to have a modest stabilizing effect for this particular design.

FIG. 8. Design pRO-2 is pH-responsive by size-exclusion chromatography (SEC), whereas design pRO-2-noHis met: SEC chromatograms using a Superdex™ 75 column and 25 mM Tris pH 8.0 at room temperature (black) or Na₂PO₄-Citrate buffer at pH 4 (red). Design pRO-2 is a soluble aggregate at pH 4 under these conditions, whereas by native mass spectrometry, pRO-2 is predominantly monomeric at pH 4 (FIG. 1C); differences could be explained by different buffer systems or the vacuum conditions of the native mass spectrometry.

FIG. 9. Reversibility of disassembly as determined by native MS. 5 μM pRO-2 and pRO-3.1 trimer were measured in 200 mM NH₄Ac (pH 6.8). Acetic acid was added to lower the pH and cause dissociation into monomers (pH 6.8→2.4). Subsequent addition of ammonia (pH 2.4→9.1) results in re-association of monomers into trimer. 6.67 μM pRO-2.3, pRO-2.4 and pRO-2.5 trimer were measured in 200 mM NH₄Ac/50 mM TEAA (pH 7.0). Acetic acid was added to decrease the pH and cause dissociation into monomers (pH 7.0→3.0). Re-association was induced via buffer-exchange to 200 mM NH₄Ac/50 mM TEAA (pH 7.0) by ultrafiltration (Amicon Ultra, MWCO 3 kDa).

FIG. 10. 1.2 Å X-ray crystal structure of design pRO-2 (PDB ID 6MSQ): (left) during refinement, positive (green) density was observed from the difference map where the proton is supposed to be in the designed hydrogen bond network. (right) The non-histidine polar network, layer l, extends to make additional hydrogen bonds with resolved water molecules as part of a very extensive hydrogen bond network.

FIG. 11. ΔG estimates (top) from GdmCl denaturation experiments (bottom); from this data, ΔG for each individual layer type (n, m, l) were estimated by solving a set of linear equations given the ΔG of folding for each design and its corresponding number of layers of each type; these values were used for the ΔG values in the theoretical model (Eq. 1) used to generate the theoretical dissociation curves in FIG. 3.

FIG. 12A-D. Small-angle X-ray scattering (SAXS) to assess flexibility. SAXS profiles of (A) designs pRO-2, pRO-2.1, pRO-2.3, pRO-2.4, pRO-2.5, and pRO-2-noHis: (B) experimental scattering data (black) at pH 8.0 is in close agreement with theoretical profiles computed from design models (red) using FoXS(41, 42); radius of gyration (Rg), maximum distance (dmax), and other metrics are also largely in agreement to the design models (Table 5). However, there are differences noticeable differences between designs that have a histidine network close to the termini (pRO-2 and pRO-2.4) compared to those that do not (pRO-2.1, pRO-2.3, pRO-2.5, and pRO-2-noHis): (C) Scaled Log 10 intensity plots (left) and Kratky plots (right) show that pRO-2 and pRO-2.4 are very similar, with spectra consistent with increased flexibility as compared to pRO-2.3 and pRO-2.5. (D) pRO-2-noHis at pH 4.0 shows subtle differences in the high q region, but is still in close agreement in the low q. Gunier region, and consistent with a trimeric species. Plots in (C) made using ScAtter™ software.

FIG. 13. Other factors that affect cooperativity; the role of the helical hairpin loop. Replacing the structured hairpin loop connecting the helices of the monomer with a flexible GS linker results in less cooperativity, as assessed by native mass spectrometry at different pH values. (left) Design pRO-2-GS loses its homogenous trimeric assembly at neutral pH when the flexible loop is introduced. (right) Design pRO-2.3.-GS retains its trimeric assembly at neutral pH, but disassembles with less cooperativity (steepness of transition) in response to lower pH than its parent design (FIG. 3D).

FIG. 14. Liposome disruption assay (as in FIG. 4) for design pRO-2 at pH 5.0 using liposomes with more native-like lipid compositions.

FIG. 15A-C. CD data for pRO-2 mutants I56V and A54M. (A-B) GdmCl denaturation experiments performed at pH 5.89 in Na₂PO₄-Citrate buffer. (A) Letting the samples sit at low pH for different amounts of time before starting experiments affected results; for this reason, all native MS and CD data at varying pH's in this study were incubated for the same short amount of time before starting each experiment to ensure consistency. (B) I56V and A54M show subtle, but reproducible, changes in stability (data shown is representative from three independent experiments). (C) Free energy of folding calculations from denaturation experiments as in FIG. 11.

FIG. 16A-B. (A) Representative electron micrographs of DOPC liposomes and purified designed proteins pRO-2 I56V and pRO-2-NoHis conjugated to 10 nm gold nanoparticles at pH 5. Free and gold conjugated pRO-2 I56V are membrane active and associate with liposomes at pH 5. Two primary modes of interaction are observed (Indicated by white arrows): liposome disruption, where the lipid bilayer appears ruptured and discontinuous, and bilayer bridging, where a tight and extended interface is formed between two liposomes. Density that likely corresponds to pRO-2 I56V can be seen at the interface. Design pRO-2 I56V does not perturb liposomes at pH 8 and the protein conjugated gold nanoparticles are well dispersed and not associated with liposomes. Design pRO-2-NoHis was similarly membrane inactive at pH 5 and 8. (B) Reconstructed cryo-electron tomograms of DOPC liposomes with designs pRO-2 I56V (left) or pRO-2-NoHis (right) at pH 5. At pH 5, pRO-2 I56V helps create extended interfaces between adjacent liposomes. Design pRO-2-NoHis does not exhibit any membrane activity at pH 5. All scale bars are 100 nm.

FIG. 17. Images of U2-OS cells loaded with LysoSensor Yellow/Blue DND-160 that are incubated with pRO-2 (5 μM, top left), pRO-2 I56V (5 μM, middle left), Untreated (bottom left), pRO-2-No His (5 μM, top right), Chloroquine (50 μM, middle right), Bafilomycin A (1 μM, bottom right) for 1 hr. Blue images represent intensities of emission acquired in the region of 410-499 nm upon 405 nm excitation. Yellow images represent intensities of emission acquired in the region of 500-600 nm upon 405 nm excitation. Intensity of excitation laser was same for all images and images are scaled to the same maximum intensity value.

FIG. 18. Normalized fluorescence measurements plotted verses pH of buffer from a fluorescent plate reader. The increase in fluorescence between pH 8.0 and 5.3 is shifted towards lower pH for the 163.2(2+1)-cpmoxCerulean3_v2 construct (cyan) compared with the (I56V)163.2(2+1)-cpmoxCerulean3_v2 construct (blue), which supports the theoretical model that reduced interface energy of hydrophobic layers (ΔG_{hydrophobic}) in the helical bundle due to the isoleucine-to-valine mutations increase the pH at which the helical bundle unfolding transition occurs. Proteins are at 5 μg/mL concentration in phosphate-citrate buffer of varying pH with 148.75 mM NaCl and 0.975 mM dithiothreitol (DTT). Data is background-subtracted from blank buffer wells. Error bars represent the standard deviation of 3 technical replicates with propagated error through analysis.

FIG. 19. Topology of de novo circularly-permuted fluorescent protein (cpFP)-based fluorescent pH biosensor construct 163.2(2+1)-cpmoxCerulean3_v2-cWSGFP2 depicted at high pH. At high pH, the helical bundle trimer (grey) is associated, and the cpmoxCeulean3_v2 (cyan) acts as a FRET donor to the C-terminal cfSGFP2(green), which acts as a FRET acceptor, producing a quantifiable FRET signal. At low pH, the helical bundle trimer dissociates due to histidine residues at the trimer interface becoming protonated, the conformational change of which is coupled to the cpmoxCerulean3_v2 FRET donor increasing in fluorescence brightness. The cpmoxCerulean3_v2 has a low pKₐ of unfolding, while the cSGFP2 has a high pKₐ of unfolding, so at low pH the cpmoxCerulean3_v2 remains folded and the cfSGFP2 unfolds reducing its ability to act as a FRET acceptor. Thus, at low pH, because the FRET donor increases in fluorescence brightness while the FRET acceptor decreases in fluorescence brightness, the overall FRET signal is reduced at low pH. The described mechanism allows the designed conformational change of the helical bundle upon pH change to be coupled to measureable fluorescence readouts.

FIG. 20A-T. pH-induced changes in oligomeric state as determined by native MS: Mass spectra are shown at the indicated pH to illustrate differences in dissociation pathways for the designs; the number of subunits in each observed oligomeric complex is denoted by n (e.g. n=3 indicates trimer, and n=1 indicates monomer). Trimers 2L6HC3_13 (A), pRO-2-noHis (B), and pRO-2.2 (E, O) show no significant pH response within pH ˜7.0 to ˜3.0. Trimers pRO-2(C, W), pRO-2.1 (D, N), pRO-2.4 (G, Q), pRO-3 (I), pRO-3.1 (J), pRO-2 I56V (S) and pRO-2 A54M (T) disassemble via tetramer as intermediate, whereas pRO-2.5 (H, R) seems to directly dissociate into monomer at low pH. pRO-2.3(F, P) forms multiple higher-order oligomers besides tetramer at low pH prior to dissociation into monomer. Dimers pRO-4 (K) and pRO-5 (L) predominantly directly dissociate into monomer at low pH. The occurrence of characteristic intermediates in pH-dependent dissociation of the designs was observed to be independent of concentration, although concentration does somewhat affect the relative percentages of the different intermediate states observed, concentrations are with respect to the initial oligomeric state at neutral pH (e.g. 5 μM pRO-2 indicates 5 μM of trimer species in the sample).

### DETAILED DESCRIPTION

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ole: I), leucine (Leu L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the disclosure provides non-naturally occurring polypeptides or polypeptide oligomers, comprising a buried hydrogen bond network that comprises at least one pH sensitive amino acid located (i) at an intra-chain interface between different structural elements in one polypeptide, or (ii) at an inter-chain interface between structural elements present in different chains of a polypeptide oligomer, wherein the polypeptide or polypeptide oligomer is stable above a given pH, and wherein the polypeptide or polypeptide oligomer undergoes a conformational transition when subjected to a pH at or below the given pH.

As disclosed in the examples, the inventors present a general strategy to design pH-polypeptides or polypeptide oligomers by precisely pre-organizing histidine residues in buried hydrogen bond networks that span across the polypeptide interface or oligomeric interface. The pH range at which disassembly occurs, as well as the cooperativity of the transition, can be programmed by balancing the number of histidine-containing networks and the strength of the surrounding hydrophobic interactions. In non-limiting embodiments, the polypeptides or polypeptide oligomers (including but not limited to homotrimers and heterodimers) are stable above pH 6.5, but undergo cooperative, large-scale conformational transitions when the pH is lowered and electrostatic and steric repulsion builds up as the network histidines involved in the buried hydrogen bond network become protonated. The repeating geometric cross-sections allow hydrogen bond networks to be added or subtracted in a modular fashion.

In one embodiment, the pH sensitive amino acids are selected from the group consisting of histidine, aspartate, and glutamate residues. In a specific embodiment, the pH sensitive amino acids comprise histidine residues.

In other embodiments, the buried hydrogen bond network comprises at least 2, 3, 4, 5, 6, or more pH sensitive amino acids.

The polypeptides or polypeptide oligomers may include any suitable “structural element”. In non-limiting embodiments, the different structural elements are selected from the group consisting of loops, beta sheets, alpha helices, or combinations thereof. In a specific embodiment the structural elements comprise alpha helices.

In another embodiment, the polypeptides or polypeptide oligomers may include at leas 2, 3, 4, 5, 6, 7, 8, 9, or more structural elements. The different structural elements in a given polypeptide or polypeptide oligomer may comprise different structural elements linked via an amino acid linker, or different structural elements present on separate polypeptides present in a polypeptide oligomer.

In one embodiment, the at least one pH sensitive amino acid located is at an intra-chain interface between different structural elements in the polypeptide. In another embodiment, the at least one pH sensitive amino acid located is at an inter-chain interface between structural elements present in different chains of the polypeptide oligomer.

In one embodiment, the buried hydrogen-bond network comprises one or more histidine-containing layers, wherein each histidine N_{ε} and N_{δ} atoms are hydrogen-bonded across the one or more interfaces.

As used herein, “layers” refer to an interaction between different structural elements in the polypeptide or polypeptide oligomer. The interaction(s) may comprise hydrogen-bonding between different structural elements, hydrophobic interactions between different structural elements, or combinations thereof.

In some embodiments, the polypeptide or polypeptide oligomer comprises a polypeptide monomer, as described herein (i.e.: the buried hydrogen bond network comprises at least one pH sensitive amino acid is located at an intra-chain interface between different structural elements in one polypeptide). In another embodiment, the polypeptide or polypeptide oligomer comprises a homo-oligomer, including but not limited to homo-trimers, or a hetero-oligomer, including but not limited to hetero-dimers as described herein (i.e.: the buried hydrogen bond network comprises at least one pH sensitive amino acid located at an inter-chain interface between structural elements present in different chains of the polypeptide oligomer).

In another embodiment, the disclosure provides non-naturally occurring pH-responsive polypeptides, comprising an oligomeric helical bundle comprising at least four alpha-helical subunits, wherein the oligomeric helical bundle comprises

one or more interfaces; and

one or more histidine-containing layers that participate in buried hydrogen bond networks, wherein each histidine N_{ε} and N_{δ} atoms are hydrogen-bonded across the one or more interfaces;

wherein the polypeptide is stable above a given pH, and wherein oligomers (including but not limited to dimers or trimers) of the polypeptide undergo a conformational transition when subjected to a pH at or below the given pH.

As will be understood by those of skill in the art, the helical bundle will include the alpha-helical subunits and a single hairpin loop per subunit; as used herein, a “helical bundle subunit” includes the alpha-helix and the hairpin loop.

In one embodiment, each alpha helix is connected to the next helix along the primary amino acid sequence via an amino acid linker. The linker may be any suitable amino acid length and composition. In various embodiments, the amino acid linker is between 4-8, 4-7, 5-8, 5-7, or 5-6 amino acids in length. Each inner helix can connect to an outer helix through a short designed loop to produce helix-turn-helix monomer subunits. The short designed loop may be any polypeptide sequence or domain that permits formation of the alpha-helical hairpin, including any functional domain insertions of interest.

In one embodiment, the polypeptide comprises two or more (i.e.: 2, 3, 4, 5, 6, or more) histidine-containing layers.

In one embodiment, the given pH is between about pH 4.5 to about pH 6.5. As described below, modification of hydrophobic layers shift the “given pH” transition point lower. As the number of hydrophobic layers increases, therefore the number of hydrophobic layers modulates the pH-responsiveness. Thus, the number of hydrophobic layers can be modified to tune pH responsiveness as deemed appropriate for an intended use.

In one embodiment, polypeptide comprises a polypeptide of formula I:

X1-X2-X3-X4-X5-X6-X7-X8-X9-X10-X11-X12-X13-X14-X15-X16-X17, wherein:

X1 and X17 are independently absent or comprise peptides;

X2, X4, X6, X8, X10, X12, X14, and X16 are each 1-2 amino acids that may be comprised of either hydrophobic residues or polar residues, forming a helical secondary structure, wherein at least 1, 2, 3, 4, 5, 6, 7, or all 8 of X2, X4, X6, X8, X10, X12, X14, and X16 include a histidine residue;

X3, X5, X7, X11, X13, and X15 Se 5-6 residue variable amino acid linkers forming a helical secondary structure; and

X9 comprises a loop, including but not limited to a hairpin loop, of variable amino acids.

The polypeptides are thus composed of a helix-loop-helix secondary structure and hairpin-shaped tertiary structure.

In this embodiment, X2, X4, X6, and X8, X10, X12, X14, and X16 are always buried in the oligomeric interface upon homo-trimerization of the polypeptide. Since a canonical alpha-helix has ˜3.6 residues per 360 degree turn, the residues in X2, X4, X6, and X8, as well as X10, X12, X14, and X16 are defined every two complete turns of the alpha-helix (i.e. since they are each 1-2 amino acids in length and domains X3, X5, X7, X11, X13, and X15 segments contain the 5-6 intervening residues. In this embodiment, the buried hydrogen bond network comprises at least one pH sensitive His residue. The polypeptides of this embodiment form homotrimers as described in the examples that follow. In this embodiment, domains X8 and X10, X6 and X12, X4 and X14, and X2 and X16 segment pairs interact in the homo-trimer to form part of a single “layer” (i.e.: the interaction between domains X8 and X10 constitutes one layer; the interaction between domains X6 and X12 constitutes a second layer, the interaction between domains X4 and X14 constitutes a third layer, and the interaction between domains X2 and X16 constitutes a fourth layer). The interactions in each layer may comprise purely hydrophobic interactions, a mix of hydrophobic and polar interactions, and/or a mix of hydrophobic and His interactions. The interactions may occur at an inter-chain interface between domains present in different subunits of the polypeptide oligomer, at an intra-chain interface between different domains in one polypeptide subunit, or both. In one embodiment, the interactions primarily may occur at an inter-chain interface between domains present in different subunits of the polypeptide oligomer.

As will be understood by those of skill in the art based on the teachings herein, other embodiments are possible and described below. For example, other polypeptides or polypeptide oligomers (including homo-trimers) may comprise 1, 2, 3 or 4 such layers. Increased numbers of such layers are also possible.

In another embodiment, the polypeptide comprises a polypeptide of formula 2:

X6-X7-X8-X9-X10-X11-X12, wherein;

X6-X8 form a first helical secondary structure;

X10-X12 form a second helical structure;

X9 comprises a loop of variable amino acid length and sequence; and

wherein at least 1, 2, 3, 4, 5, or all 6 of X6, X7, X8, X10, X11, and X12 include a pH sensitive amino acid residue;

wherein the polypeptide or an oligomer comprising the polypeptide undergoes a conformational transition when subjected to a pH at or below a given pH.

In a further embodiment, the polypeptide comprises a polypeptide of formula 3:

X4-X5-X6-X7-X8-X9-X10-X11-X12-X13-X14, wherein;

X4-X8 form a first helical secondary structure;

X10-X14 form a second helical structure;

X9 comprises a loop of variable amino acid length and sequence; and

wherein at least 1, 2, 3, 4, 5, 6, 7, 8, 9, or all 10 of X4, X5, X6, X7, X8, X10, X11, X12, X13, and X14 include a pH sensitive amino acid residue;

wherein the polypeptide or an oligomer comprising the polypeptide undergoes a conformational transition when subjected to a pH at or below a given pH.

In another embodiment, the polypeptide comprises a polypeptide of formula 4:

X2-X3-X4-X5-X6-X7-X8-X9-X10-X11-X12-X13-X14-X15-X16, wherein:

X2-X8 form a first helical secondary structure;

X10-X16 form a second helical structure;

X9 comprises a loop of variable amino acid length and sequence and

wherein at least 1, 2, 3, 4, 5, 6, 7, 8, 9, 10, 11, 12, 13, or all 14 of X2, X3, X4, X5, X6, X7, X8, X10, X11, X12, X13, X14, X15, and X16 include a pH sensitive amino acid residue;

wherein the polypeptide or an oligomer comprising the polypeptide undergoes a conformational transition when subjected to a pH at or below a given pH.

In each of these embodiments, the polypeptide, or polypeptide oligomers comprising the polypeptide comprise a buried hydrogen bond network that comprises at least one pH sensitive amino acid located (i) at an intra-chain interface between different domains in one polypeptide, or (ii) at an inter-chain interface between domains present in different chains of a polypeptide oligomer, wherein the polypeptide or polypeptide oligomer is stable above a given pH, and wherein the polypeptide or polypeptide oligomer undergoes a conformational transition when subjected to a pH at or below the given pH.

In one embodiment, the pH sensitive amino acids are selected from the group consisting of histidine, aspartate, and glutamate residues. In a specific embodiment, the pH sensitive amino acids comprise histidine residues.

In other embodiments, the buried hydrogen bond network comprises at least 2, 3, 4, 5, 6, or more pH sensitive amino acids.

The various X domains in these embodiments may comprise any length or content of amino acids so long as the recited limitations are met. In one embodiment of any of these embodiments, 1, 2, 3, 4, 5, 6, 7, or all 8 of X2, X4, X6, X8, X10, X12, X14, and X16 (when present) are 1-2 amino acids that may be comprised of hydrophobic residues, polar residues or both, wherein at least 1, 2, 3, 4, 5, 6, 7, or all 8 of X2, X4, X6, X8, X10, X12, X14, and X16 (when present) include a pH sensitive amino acid.

In another embodiment that can be combined with any of these embodiments, 1, 2, 3, 4, 5, or all 6 of X3, X5, X7, X11, X13, and X15 (when present) are 5-6 residue variable amino acid linkers.

In a further embodiment of any of these embodiments, X9 nay comprise a hairpin loop, or may comprise a flexible linker including but not limited to a flexible GS-based linker.

In a further embodiment of any of these embodiments, additional amino acid residues or functional domains may be present, such as at the N- or C-terminus, as deemed appropriate for an intended use.

As used herein, amino acid residues in a polar layer could be any of the following: C, D, E, G, K, N, Q, R, S, T, Y, W, and H. Amino acid residues in a hydrophobic layer could be any of the following: A, F, G, I, L, M, P, V, W and norleucine.

Hydrophobic layers shift the “given pH” transition point lower as the number of hydrophobic layers increases, therefore the number of hydrophobic layers does modulate the pH-responsiveness. Thus, the number of hydrophobic layers can be modified to tune pH responsiveness as deemed appropriate for an intended use.

In one embodiment, 1, 2, 3, 4, 5, 6, or 7 of X2, X4, X6, X8, X10, X12, X14, and X16 are comprised of hydrophobic residues, as deemed suitable for an intended use. For example, to shift the “given pH” lower, the number of hydrophobic domains is increased and the number of polar domains is decreased; to shift the “given pH” higher, the number of hydrophobic domains is decreased and the number of polar domains is increased.

In another embodiment X9 is at least 2, 3, 4, 5, 6, 7, 8, 9, 10, 11, 12, 13, 14, 15, 16, 17, 18, 19, 20, 21, 22, 23, 24, 25 or more amino acids in length.

In a further embodiment, each of X1 and X17, when present, are the same length.

In one embodiment, one or more of X1, X9 and X17 comprise a functional subunit, or the polypeptide further comprises a functional domain at the N-terminus or C-terminus. A “functional subunit” is any domain that can be add functionality to the polypeptide. Any functional domain may be used as suitable for an intended purpose. In one embodiment, the functional subunit comprises a detectable protein or functional fragment thereof, including but not limited to a fluorescent protein or functional fragment thereof. For example, a functional subunit comprising a fluorescent protein or functional fragment thereof permits coupling of the conformational change due to protonation of the buried histidines in the hydrogen bond networks at the interface of the helical bundle to conformational changes in the chromophore environment of the fused fluorescent protein. This provides a fluorescent readout of the conformation change. As will be understood by those of skill in the art, other functional subunits could be used in a similar manner to link the pH-based conformational change with a readout based on the function of the functional subunit.

In another embodiment, the polypeptide comprises the amino acid sequence at least 25%, 30%, 35%, 40%, 50%, 55%, 60%, 65%, 70%, 75%, 80%, 85%, 90%, 95%, 96%, 97%, 98%, 99%, or 100% identical to a polypeptide selected from the group consisting of SEQ ID NOs: 1-40, 45-46, 60-66, 69-76 and 81-86.

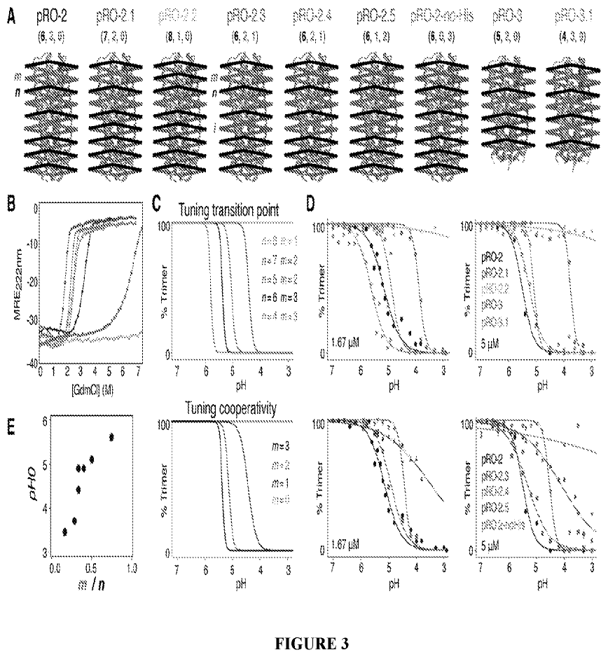

TABLE 1
In this table, the bold residues show the
differences between the modular designs of
FIG. 3 in the manuscript, which allows
mapping of how the layers can be swapped.
Underlined region is not part of the
design but hexahistidine tag and TEV
cleavage site for purification
((i.e.: the residues are optional)

| | |
|---|---|
| pRO-2 | MGSHHHHHHGSGSENLYFQGSEYEIRKALEELKAATAE |
| | LKRATASLRAITEELKKNPSEDALVEHNRAIVEHNAII |
| | VEHNRIIAAVLELIVRAIE (SEQ ID NO: 1) |
| | SEYEIRKALEELKAATAELKRATASLRAITEELKKNPS |
| | EDALVEHNRAIVEHNAIIVEHNRIIAAVLELIVRAIK |
| | (SEQ ID NO: 2) |
| pRO- | MGSHHHHHHGSGSENLYFQGSEYEIRKALEELKAALAE |
| 2.1 | LKRATASLRAITEELKKNPSEDALVEHNRAIVEHNAII |
| | VEVLRIIAAVLELIVRAIE (SEQ ID NO: 3) |
| | SEYEIRKALEELKAALAELKRATASLRAITEELKKNPS |
| | EDALVEHNRAIVEHNAIIVEVLRIIAAVLELIVRAIK |
| | (SEQ ID NO: 4) |
| pRO- | MGSHHHHHHGSGSENLYFQGSEYEIRKALEELKAALAE |
| 2.2 | LKRATASLRAILEELKKNPSEDAIVEAIRAIVEHNAII |
| | VEVLRIIAAVLELIVRAIE (SEQ ID NO: 5) |
| | SEYEIRKALEELKAALAELKRATASLRAILEELKKNPS |
| | EDAIVEAIRAIVEHNAIIVEVLRIIAAVLELIVRAIE |
| | (SEQ ID NO: 6) |
| pRO- | MGSHHHHHHGSGSENLYFQGSEYEIRKALEELKASTAE |
| 2.3 | LKRATASLRAITEELKKNPSEDALVEHNRAIVEHNAII |
| | VENNRIIAAVLELIVRAIE (SEQ ID NO: 7) |
| | SEYEIRKALEELKASTAELKRATASLRAITEELKKNPS |
| | EDALVEHNRAIVEHNAIIVENNRIIAAVLELIVRAIE |
| | (SEQ ID NO: 8) |
| pRO- | MGSHHHHHHGSGSENLYFQGSEYEIRKALEELKAATAE |
| 2.4 | LKRATASLRASTEELKKNPSEDALVENNRLIVEHNAII |
| | VEHNRIIAAVLELIVRAIE (SEQ ID NO: 9) |
| | SEYEIRKALEELKAATAELKRATASLRASTEELKKNPS |
| | EDALVENNRLIVEHNAIIVEHNRIIAAVLELIVRAIE |
| | (SEQ ID NO: 10) |
| pRO- | MGSHHHHHHGSGSENLYFQGSEYEIRKALEELKASTAE |
| 2.5 | LKRATASLRASTEELKKNPSEDALVENNRLIVEHNAII |
| | VENNRIIAAVLELIVRAIE (SEQ ID NO: 11) |
| | SEYEIRKALEELKASTAELKRATASLRASTEELKKNPS |
| | EDALVENNRLIVEHNAIIVENNRIIAAVLELIVRAIE |
| | (SEQ ID NO: 12) |

TABLE 2
In this table:
Histidine-containing hydrogen bond network
residues are bolded
Non-histidine hydrogen bond network
residues are highlighted and underlined
Longer underlined region is not part of the deisgn
but hexahistidine tag and TEV cleavage site for
purification (i.e., it is optional).

| | |
|---|---|
| pRO-2 | MGSHHHHHHGSGSENLYFQGSEYEIRKALEELKAATAE |
| | LKRATASLRAITEELKKNPSEDALVEHNRAIVEHNAII |
| | VEHNRIIAAVLELIVRAIK (SEQ ID NO: 1) |
| | SEYEIRKALEELKAATAELKRATASLRAITEELKKNPS |
| | EDALVEHNRAIVEHNAIIVEHNRIIAAVLELIVRAIK |
| | (SEQ ID NO: 2) |
| pRO- | MGSHHHHHHGSGSENLYFQGSEYEIRKALEELKAALAE |
| 2.1 | LKRATASLRAITEELKKNPSEDALVEHNRAIVEHNAII |
| | VEVLRIIAAVLELIVRAIK (SEQ ID NO: 3) |
| | SEYEIRKALEELKAALAELKRATASLRAITEELKKNPS |
| | EDALVEHNRAIVEHNAIIVEVLRIIAAVLELIVRAIK |
| | (SEQ ID NO: 4) |
| pRO- | MGSHHHHHHGSGSENLYFQGSEYEIRKALEELKAALAE |
| 2.2 | LKRATASLRAILEELKKNPSEDALVEAIRAIVEHNAII |
| | VEVLRIIAAVLELIVRAIK (SEQ ID NO: 5) |
| | SEYEIRKALEELKAALAELKRATASLRAILEELKKNPS |
| | EDAIVEAIRAIVEHNAIIVEVLRIIAAVLELIVRAIK |
| | (SEQ ID NO: 6) |
| pRO- | MGSHHHHHHGSGSENLYFQGSEYEIRKALEELKASTAE |
| 2.3 | LKRATASLRAITEELKKNPSEDALVEHNRAIVEHNAII |
| | VENNRIIAAVLELIVRAIK (SEQ ID NO: 7) |
| | SEYEIRKALEELKASTAELKRATASLRAITEELKKNPS |
| | EDALVEHNRAIVEHNAIIVENNRIIAAVLELIVRAIK |
| | (SEQ ID NO: 8) |
| pRO- | MGSHHHHHHGSGSENLYFQGSEYEIRKALEELKAATAE |
| 2.4 | LKRATASLRASTEELKKNPSEDALVENNRAIVEHNAII |
| | VEHNRIIAAVLELIVRAIK (SEQ ID NO: 9) |
| | SEYEIRKALEELKAATAELKRATASLRASTEELKKNPS |
| | EDALVENNRLIVEHNAIIVEHNRIIAAVLELIVRAIK |
| | (SEQ ID NO: 10) |
| pRO- | MGSHHHHHHGSGSENLYFQGSEYEIRKALEELKASTAE |
| 2.5 | LKRATASLRASTEELKKNPSEDALVENNRLIVEHNAII |
| | VENNRIIAAVLELIVRAIK (SEQ ID NO: 11) |
| | SEYEIRKALEELKASTAELKRATASLRASTEELKKNPS |
| | EDALVENNRLIVEHNAIIVENNRIIAAVLELIVRAIK |
| | (SEQ ID NO: 12) |

TABLE 3
Amino acid sequences of all designs tested. All constructs were
cloned into pET21-NESG plasmid except for design pRO-1, which was cloned in
PET28b. Heterodimers pRO-4 and pRO-5 were ordered as [symbol]
constructs; DNA sequence containing stop codon, additional ribosome binding
sequence, and second start codon is shown by the lower case letters in
parenthesis (this sequence is not included in the amino acid sequence or
associate SEQ ID NO). Underlined regions are removed after hexahistidine
tag cleavage (i.e.: they are optional). Bold positions indicate
mutations/differences between a design variant and its parent design.

| Design name | Amino acid sequences of designed proteins in this study |
|---|---|
| pRO-1 | MGSSHHHHHHSSGLVPRGSHMGTLKEVLERLEEVLRRHREVAREHQRWAREHEQWVRDDP |
| | NSAKWIAESTRWILETTDAISRTADVLAEAIRVLAESD (SEQ ID NO: 13) |
| | GSHMGTLKEVLERLEEVLRRHREVAREHQRWAREHEQWVRDDPNSAKWIAESTRWILETT |
| | DAISRTADVLAEAIRVLAESD (SEQ ID NO: 14) |
| pRO-2 | MGSHHHHHHGSGSENLYFQGSEYEIRKALEELKAATAELKRATASLRAITEELKKNPSED |
| | ALVEHNRAIVEHNAIIVEHNRIIAAVLELIVRAIK (SEQ ID NO: 1) |
| | SEYEIRKALEELKAATAELKRATASLRAITEELKKNPSEDALVEHNRAIVEHNAIIVEHN |
| | RIIAAVLELIVRAIK (SEQ ID NO: 2) |
| pRO-2 H45N/ | MGSHHHHHHGSGSENLYFQGSEYEIRKALEELKAATAELKRATASLRAITEELKKNPSED |
| H52N/H59N | ALVENNRAIVENNAIIVENNRIIAAVLELIVRAIK (SEQ ID NO: 15) |
| | GSEYEIRKALEELKAATAELKRATASLRAITEELKKNPSEDALVENNRAIVENNAIIVEN |
| | NRIIAAVLELIVRAIK (SEQ ID NO: 16) |
| pRO-2- | MGSHHHHHHGSGSENLYFQGSEYEIRKALEELKASTAELKRSTASLRASTEELKKNPSED |
| noHis | ALVENNRLIVENNAIIVENNRIIAAVLELIVRAIK (SEQ ID NO: 75) Inactive |
| | control |
| | GSEYEIRKALEELKASTAELKRSTASLRASTEELKKNPSEDALVENNRLIVENNAIIVEN |
| | NRIIAAVLELIVRAIK (SEQ ID NO: 76) Inactive control |
| pRO-2.1 | MGSHHHHHHGSGSENLYFQGSEYEIRKALEELKAALAELKRATASLRAITEELKKNPSED |
| | ALVEHNRAIVEHNAIIVEVLRIIAAVLELIVRAIK (SEQ ID NO: 3) |
| | SEYEIRKALEELKAALAELKRATASLRAITEELKKNPSEDALVEHNRAIVEHNAIIVEVL |
| | RIIAAVLELIVRAIK (SEQ ID NO: 4) |
| pRO-2.2 | MGSHHHHHHGSGSENLYFQGSEYEIRKALEELKAALAELKRATASLRAILEELKKNPSED |
| | AIVEAIRAIVEHNAIIVEVLRIIAAVLELIVRAIK (SEQ ID NO: 5) |
| | SEYEIRKALEELKAALAELKRATASLRAILEELKKNPSEDAIVEAIRAIVEHNAIIVEVL |
| | RIIAAVLELIVRAIK (SEQ ID NO: 6) |
| pRO-2.3 | MGSHHHHHHGSGSENLYFQGSEYEIRKALEELKASTAELKRATASLRAITEELKKNPSED |
| | ALVEHNRAIVEHNAIIVENNRIIAAVLELIVRAIK (SEQ ID NO: 7) |
| | SEYEIRKALEELKASTAELKRATASLRAITEELKKNPSEDALVEHNRAIVEHNAIIVENN |
| | RIIAAVLELIVRAIK (SEQ ID NO: 8) |
| pRO-2.4 | MGSHHHHHHGSGSENLYFQGSEYEIRKALEELKAATAELKRATASLRASTEELKKNPSED |
| | ALVENNRLIVEHNAIIVEHNRIIAAVLELIVRAIK (SEQ ID NO: 9) |
| | SEYEIRKALEELKAATAELKRATASLRASTEELKKNPSEDALVENNRLIVEHNAIIVEHN |
| | RIIAAVLELIVRAIK (SEQ ID NO: 10) |
| pRO-2.5 | MGSHHHHHHGSGSENLYFQGSEYEIRKALEELKASTAELKRATASLRASTEELKKNPSED |
| | ALVENNRLIVEHNAIIVENNRIIAAVLELIVRAIK (SEQ ID NO: 11) |
| | SEYEIRKALEELKASTAELKRATASLRASTEELKKNPSEDALVENNRLIVEHNAIIVENN |
| | RIIAAVLELIVRAIK (SEQ ID NO: 12) |
| pRO-2 I56V | MGSHHHHHHGSGSENLYFQGSEYEIRKALEELKAATAELKRATASLRAITEELKKNPSED |
| | ALVEHNRAIVEHNAIVVEHNRIIAAVLELIVRAIK (SEQ ID NO: 17) |
| | GSEYEIRKALEELKAATAELKRATASLRAITEELKKNPSEDALVEHNRAIVEHNAIVVEH |
| | NRIIAAVLELIVRAIK (SEQ ID NO: 18) |
| pRO-2 A54M | MGSHHHHHHGSGSENLYFQGSEYEIRKALEELKAATAELKRATASLRAITEELKKNPSED |
| | ALVEHNRAIVEHNMIIVEHNRIIAAVLELIVRAIK (SEQ ID NO: 19) |
| | GSEYEIRKALEELKAATAELKRATASLRAITEELKKNPSEDALVEHNRAIVEHNMIIVEH |
| | NRIIAAVLELIVRAIK (SEQ ID NO: 20) |
| pRO-2 I70N | MGSHHHHHHGSGSENLYFQGSEYEIRKALEELKAATAELKRATASLRAITEELKKNPSED |
| | ALVEHNRAIVEHNAIIVEHNRIIAAVLELNVRAIK (SEQ ID NO: 21) |
| | GSEYEIRKALEELKAATAELKRATASLRAITEELKKNPSEDALVEHNRAIVEHNAIIVEH |
| | NRIIAAVLELNVRAIK (SEQ ID NO: 22) |
| pRO-3 | MGSHHHHHHGSGSENLYFQGSEALYELEKALRELKKATAALERATAELKKNPSEDALVEH |
| | NRLIAAHNKIIAEVLRIIAKVLK (SEQ ID NO: 23) |
| | GSEALYELEKALRELKKATAALERATAELKKNPSEDALVEHNRLIAAHNKIIAEVLRIIA |
| | KVLK (SEQ ID NO: 24) |
| pRO-3.1 | MGSHHHHHHGSGSENLYFQGSEALYELEKATRELKKATDELERATEELEKNPSEDALVEH |
| | NRLIAEHNKIIAEHNRIIAKVLK (SEQ ID NO: 25) |
| | GSEALYELEKATRELKKATDELERATEELEKNPSEDALVEHNRLIAEHNKIIAEHNRIIA |
| | KVLK (SEQ ID NO: 26) |
| pRO-4 | MDEEDHLKKLKTHLEKLERHLKLLEDHAKKLEDILKERPEDSAVKESIDELRRSIELVRE |
| internal | SIEIFRQSVEEEE(taagaaggagatatcatcatg)GSSHHHHHHSSGENLYFQGDVKEL |
| ribosome | TKILDTLTKILETATKVIKDATKLLEEHRKSDKPDPRLIETHKKLVEEHETLVRQHKELA |
| binding | EEHLKRTR (SEQ ID NO: 27) |
| site | MDEEDHLKKLKTHLEKLERHLKLLEDHAKKLEDILKERPEDSAVKESIDELRRSIELVRE |
| | SIEIFRQSVEEEE(taagaaggagatatcatcatg)GDVKELTKILDTLTKILETATKVI |
| | KDATKLLEEHRKSDKPDPRLIETHKKLVEEHETLVRQHKELAEEHLKRTR (SEQ ID |
| | NO: 28) |
| | Chain A |
| | MDEEDHLKKLKTHLEKLERHLKLLEDHAKKLEDILKERPEDSAVKESIDELRRSIELVRE |
| | SIEIFRQSVEEEE (SEQ ID NO: 81) |
| | Chain B |
| | GSSHHHHHHSSGENLYFQGDVKELTKILDTLTKILETATKVIKDATKLLEEHRKSDKPDP |
| | RLIETHKKLVEEHETLVRQHKELAEEHLKRTR (SEQ ID NO: 82) |
| pRO-4 L23A/ | MDEEDHLKKLKTHLEKLERHLKLAEDHAKKLEDILKERPEDSAVKESIDELRRSIELVRE |
| V130A | SIEIFRQSVEEEE(taagaaggagatatcatcatg)GSSHHHHHHSSGENLYFQGDVKEL |
| | TKILDTLTKILETATKVIKDATKLLEEHRKSDKPDPRLIETHKKLVEEHETLARQHKELA |
| | EEHLKRTR (SEQ ID NO: 29) |
| | MDEEDHLKKLKTHLEKLERHLKLAEDHAKKLEDILKERPEDSAVKESIDELRRSIELVRE |
| | SIEIFRQSVEEEE(taagaaggagatatcatcatg)GDVKELTKILDTLTKILETATKVI |
| | KDATKLLEEHRKSDKPDPRLIETHKKLVEEHETLARQHKELAEEHLKRTR (SEQ ID |
| | NO: 30) |
| | MDEEDHLKKLKTHLEKLERHLKLAEDHAKKLEDILKERPEDSAVKESIDELRRSIELVRE |
| | SIEIFRQSVEEEE (SEQ ID NO: 83) |
| | GSSHHHHHHSSGENLYFQGDVKELTKILDTLTKILETATKVIKDATKLLEEHRKSDKPDP |
| | RLIETHKKLVEEHETLARQHKELAEEHLKRTR (SEQ ID NO: 84) |
| pRO-5 | MTKEDILERQRKIIERAQEIHRRQQEILKEQEKIIRKPGSSEEAMKRSLKLIEESLRLLK |
| | ELLELSEESAQLLYEQR(taagaaggagatatcatcatgGSSHHHHHHSSGENLYFQGTE |
| | KRLLEEAERAHREQKEIIKKAQELHKELTKIHQQSGSSEEAKKRALKISQEIRELSKRSL |
| | ELLREILYLSQEQK (SEQ ID NO: 31) |
| | MTKEDILERQRKIIERAQEIHRRQQEILKEQEKIIRKPGSSEEAMKRSLKLIEESLRLLK |
| | ELLELSEESAQLLYEQR(taagaaggagatatcatcatg)GTEKRLLEEAERAHREQKEI |
| | IKKAQELHKELTKIHQQSGSSEEAKKRALKISQEIRELSKRSLELLREILYLSQEQK |
| | (SEQ ID NO: 32) |
| | MTKEDILERQRKIIERAQEIHRRQQEILKEQEKIIRKPGSSEEAMKRSLKLIEESLRLLK |
| | ELLELSEESAQLLYEQR (SEQ ID NO: 85) |
| | GSSHHHHHHSSGENLYFQGTEKRLLEEAERAHREQKEIIKKAQELHKELTKIHQQSGSSE |
| | EAKKRALKISQEIRELSKRSLELLREILYLSQEQK (SEQ ID NO: 86) |
| pRO-2-GS | MGSHHHHHHGSGSENLYFQGSEYEIRKALEELKAATAELKRATASLRAITEELKKGGSGS |
| | GSEDALVEHNRAIVEHNAIIVEHNRIIAAVLELIVRAIK (SEQ ID NO: 33) |
| | GSEYEIRKALEELKAATAELKRATASLRAITEELKKGGSGSGSEDALVEHNRAIVEHNAI |
| | IVEHNRIIAAVLELIVRAIK (SEQ ID NO: 34) |
| pRO-2.3-GS | MGSHHHHHHGSGSENLYFQGSEYEIRKALEELKASTAELKRATASLRAITEELKKGGSGS |
| | GSEDALVEHNRAIVEHNAIIVENNRIIAAVLELIVRAIK (SEQ ID NO: 35) |
| | GSEYEIRKALEELKASTAELKRATASLRAITEELKKGGSGSGSEDALVEHNRAIVEHNAI |
| | IVENNRIIAAVLELIVRAIK (SEQ ID NO: 36) |

[symbol]

indicates data missing or illegible when filed

The polypeptides of SEQ ID NOS:1-26 and 33-36 all form homotrimers and the polypeptides of SEQ ID NOS:27-32 and 81-86 form heterodimers. In these embodiments, the buried hydrogen bond network comprises at least one pH sensitive amino acid located at an inter-chain interface between structural elements present in different chains of the polypeptide oligomer.

The following embodiments of the polypeptides of the disclosure (SEQ ID NOS: 37-40, 45-46, 60-66, and 69-76) are single chain monomers, and the buried hydrogen bond network comprises at least one pH sensitive amino acid is located at an intra-chain interface between different structural elements in the polypeptide. The underlined regions of the following sequences are not part of the design but hexahistidine tag and thrombin or TEV cleavage site for purification (i.e.: the underlined regions are optional). In many of these sequences the monomeric subunits of the homotrimer are fused by linkers/loops and function domains into a single polypeptide sequence

pRO2.3, single-chain, with GS linkers on all the loops, asymmetrized, and a TEV site opposite to the termini direction. This allows the pH responsive trimer to be fused at its n-terminus to other proteins, such as a nanoparticle, and confer membrane disruption. Based on the liposome assay described below, the kinetics of dissociation of linked-pH trimer is slower but achieves the same membrane disruption levels as measured by dye leakage over time (on the order of minutes). This performs as well as pRO2.3 as measured by the liposome disruption assay in the context of a nanoparticle (i.e. fused at its n-terminus to a nanoparticle).

(SEQ ID NO: 37)
GSEEEIKRLLEELRKSSEELRRITKELDDLSKELRVGGSGSGSEMLVEH
NKLISEHNRTIVENNRIIVEILEAIARVGGSGSGSVEVERILDELRKSS
EELDRVTKELKKLTEELDVGGSENLYFQGSGSVEALVRHNVLITRHNDI
IVKNNDIINKILKLIAEAVGGSGSGSELERILRELEESTKELRKATEEL
RRLSEELKVGGSGSGSVEALVRHNEAIVEHNKIIVKNNDIIVKILELIT
ERI

The next polypeptide is similar to pRO2.3, with the TEV site parallel to the termini such that a monomer is released upon cleavage. This monomer is modified to have aromatic residues (phenylalanine and tryptophan) on the n-terminal helix to enhance membrane disruption. This performs slightly (5-10%) better than the pRO2.3 homotrimer in the liposome disruption assay.

(SEQ ID NO: 38)
GSEEEIKRLLEELRKSSEELRRITKELDDLSKELRVGGSGSGSEMLVEH
NKLISEHNRIIVENNRIIVEILEAIARVGGSGSGSVEVERILDELRKSS
EELDRVTKELKKLTEELDVGGSGSGSVEALVRHNVLITRHNDIIVKNND
IINKILKLIGEAVGGSENLYFQGSGSEFERWLRQLEESTKELRKFTEEL
RRFSEELKVGGSGSGSVEALVRHNEAIVEHNKAIVKNNDIIVKILELVT
ERI

Similar to pRO2.3, with Thrombin cleavage sites on each loop opposite to the termini. Also has the destabilizing I56V mutation to shift the pH disassembly to a higher pH. This performs close as well as pRO2.3 as measured by the liposome disruption assay in the context of a nanoparticle (i.e., fused at its n-terminus to a nanoparticle) but with slower kinetics.

(SEQ ID NO: 39)
GSEEEIKRLLEELRKSSEELRRITKELDDLSKELRVGGSGSGSLVPRGS
GSGSGSHALVEHNKLISEHNRIVVENNRIIVEILEAIARVGGSGSGSVE
VERILDELRKSSEELDRVTKELKKLTEELDVGGSGSGSLVPRGSGSGSG
SVEALVRHNVLITRHNDIVVKNNDIINKILKLIAEAVGGSGSGSELERI
LRELEESTKELRKATEELRRLSEELKVGGSGSGSLVPRGSGSGSGSHEA
LVRHNEAIVEHNKIVVKNNDIIVKILELITERI

Same as above, but with the third asparagine network mutated such that it is all hydrophobics to destabilize the linked-trimer and increase hydrophobic content for better membrane interaction. This performs 5-10% better than pRO2.3 as measured by the liposome disruption assay in the context of a nanoparticle (i.e., fused at its n-terminus to a nanoparticle) but with slower kinetics.

(SEQ ID NO: 40)
GSEEEIKRLLEELRKALEELRRITKELDDLSKELRVGGSGSGSLVPRGS
GSGSGSHALVEHNKLISEHNRIVVEVLRIIAEILEAIARVGGSGSGSVE
VERILDELRKALEELDRVTKELKKLTEELDVGGSGSGSLVPRGSGSGSG
SVEALVRHNVLITRHNDIVVKVLDIIAKILKLIAEAVGGSGSGSELERI
LRELEEALKELRKATEELRRLSEELKVGGSGSGSLVPRGSGSGSGSHEA
LVRHNEAIVEHNKIVVKVLDIIAKILELITERI

Additional polypeptides of the disclosure and inactive controls (i.e.: not pH responsive) are shown below. Underlined residues and/or residues in parentheses are optional.

single_chain_noHis_asym_163
(SEQ ID NO: 41)
(MGSSHHHHHHSSGLVPRGS)HMGSDELKYELEKSTRELQKSTDELEKSTEELERNPSKDVLVENNELIVRNNKI
IVKNNIIIVRTEKKGSGGSGDELKEELEKSTRELDKSTKKLERSTEELKRNPSKDALVENNKLIVENNTIIVRNN
DIIVRTRKKGSGGSGDELKEELEKSTRELKKSTKELQKSTEELERNPSKDALVKNNKLIADNNRIIVRNNTIIVR
DIKAS
Inactive control
single_chain_noHis_asym_163
(SEQ ID NO: 42)
HMGSDELKYELEKSTRELQKSTDELEKSTEELERNPSKDVLVENNELIVRNNKIIVKNNIIIVRTEKKGSGGSGD
ELKEELEKSTRELDKSTKKLERSTEELKRNPSKDALVENNKLIVENNTIIVRNNDIIVRTRKKGSGGSGDELKEE
LEKSTRELKKSTKELQKSTEELERNPSKDALVKNNKLIADNNRIIVRNNTIIVRDIKAS
Inactive control
single_chain_noHis_asym_162
(SEQ ID NO: 43)
(MGSSHHHHHHSSGLVPRGS)HMGSDDEDLDRVLEELRRSTEELDRSTKDLERSTQELRRNPSVDALVKNNNAIV
RNNEIIVENNRIILEVLELLLRSIKGSGGSGDREEIKKVLDELRESTERLERSTEELRRSTEELKKNPAVEVLVR
NNTIIVKNNKIIVDNNRIIVRVLELLEKTIKGSGGSGDKYEIRKVLKELKDSTEELRNSTKNLTDSTEELKRNPS
VEILVKNNILIVENNKIIVENNRIIVDVLELIRKAIAS
Inactive control
single_chain_noHis_asym_162
(SEQ ID NO: 44)
HMGSDDEDIDRVLEELRRSTEELDRSTKDLERSTQELRRNPSVDALVKNNNAIVRNNEIIVENNRIILEVLELLL
RSIKGSGGSGDREEIKKVLDELRESTERLERSTEELRRSTEELKKNPAVEVLVRNNTIIVKNNKIIVDNNRIIVR
VLELLEKTIKGSGGSGDKYEIRKVLKELKDSTEELRNSTKNLTDSTEELKRNPSVEILVKNNILIVENNKIIVEN
NRIIVDVLELIRKAIAS
Inactive control
single_chain_asym_162
(SEQ ID NO: 45)
(MGSSHHHHHHSSGLVPRGS)HMGSDDEDLDRVLEELRRITEELDRITKDLERLTQELRRNPSVDALVKHNNAIV
RHNEIIVEHNRIILEVLELLLRSIKGSGGSGDREEIKKVLDELREATERLERATEELRRLTEELKKNPAVEVLVR
HNTIIVKHNKIIVDHNRIIVRVLELLEKTIKGSGGSGDKYEIRKVLKELKDITEELRNMTKNLTDLTEELKRNPS
VEILVKHNILIVEHNKIIVEHNRIIVDVLELIRKAIAS
single_chain_asym_162
(SEQ ID NO: 46)
HMGSDDEDIDRVLEELRRITEELDRITKDLERLTQELRRNPSVDALVKHNNAIVRHNEIIVEHNRIILEVLELLL
RSIKGSGGSGDREEIKKVLDELREATERLERATEELRRSTEELKKNPAVEVLVRHNTIIVKHNKIIVDHNRIIVR
VLELLEKTIKGSGGSGDKYEIRKVLKELKDITEELRNMTKNLTDLTEELKRNPSVEILVKHNILIVEHNKIIVEH
NRIIVDVLELIRKAIAS
TagGFP2-TEV-TagBFP: Two fluorescent proteins TagGFP2 and TagBFP fused
together by a TEV protease site linker.
(SEQ ID NO: 47)
(MGSSHHHHHHSSGLVPRGS)HMSGGEELFAGIVPVLIELDGDVHGHKFSVRGEGEGDADYGKLEIKFICTTGKL
PVPWPTLVTTLCYGIQCFARYPEHMKMNDFFKSAMPEGYIQERTIQFQDDGKYKTRGEVKFEGDTLVNRIELKGK
DFKEDGNILGHKLEYSFNSHNVYIRPDKANNGLEANFKTRHNIEGGGVQLADHYQTNVPLGDGPVLIPINHYLST
QTKISKDRNEARDHMVLLESFSACCHTGGSGGSENLYFQGASGGSGSELIKENMHMKLYMEGTVDNHHFKCTSEG
EGKPYEGTQTMRIKVVEGGPLPFAFDILATSFLYGSKTFINHTQGIPDFFKQSFPEGFTWERVTTYEDGGVLTAT
QDTSLQDGCLIYNVKIRGVNFTSNGPVMQKKTLGWEAFTETLYPADGGLEGRNDMALKLVGGSHLIANIKTTYRS
KKPAKNLKMPGVYYVDYRLERIKEANNETYVEQHEVAVARY
(SEQ ID NO: 48)
HMSGGEELFAGIVPVLIELDGDVHGHKFSVRGEGEGDADYGKLEIKFICTTGKLPVPWPTLVTTLCYGIQCFARY
PEHMKMNDFFKSAMPEGYIQERTIQFQDDGKYKTRGEVKFEGDTLVNRIELKGKDFKEDGNILGHKLEYSFNSHN
VYIRETKANNGLEANFKTRHNIEGGGVQLADHYQTNVPLGDGPVLIPINHYLSTQTKISKDRNEARDHMVLLESF
SACCHTGGSGGSENLYFQGASGGSGSELIKENMHMKLYMEGTVDNHHFKCTSEGEGKPYEGTQTMRIKVVEGGPL
PFAFDILATSFLYGSKTFINHTQGIPDFFKQSFPEGFTWERVTTYEDGGVLTATQDTSLQDGCLIYNVKIRGVNF
TSNGPVMQKKTLGWEAFTETLYPADGGLEGRNDMALKLVGGSHLIANIKTTYRSKKPAKNLKMPGVYYVDYRLER
IKEANNETYVEQHEVAVARY
TagGFP2-single_chain_noHis_asym_163-TagBFP
(SEQ ID NO: 49)
(MGSSHHHHHHSSGLVPRGS)HMSGGEELFAGIVFVLIELDGDVHGHKFSVRGEGEGDADYGKLEIKFICTTGKL
PVPWPTLVTTLCYGIQCFARYPEHMKMNDFFKSAMPEGYIQERTIQFQDDGKYKTRGEVKFEGDTLVNRIELKGK
DFKEDGNILGHKLEYSFNSHNVYIRPDKANNGLEANFKTRHNIEGGGVQLADHYQTNVPLGDGPVLIPINHYLST
QTKISKDRNEARDHMVLLESFSACCHTGGSGGSDELKYELEKSTRELQKSTDELEKSTEELERNPSKDVLVENNE
LIVRNNKIIVKNNIIIVRTEKKGSGGSGDELKEELEKSTRELDKSTKKLERSTEELKRNPSKDALVENNKLIVEN
NTIIVRNNDIIVRTRKKGSGGSGDELKEELEKSTRELKKSTKELQKSTEELERNPSKDALVKNNKLIADNNRIIV
RNNTIIVRDIKASGGSGSELIKENMHMKLYMEGTVDNHHFKCTSEGEGKPYEGTQTMRIKVVEGGPLPFAFDILA
TSFLYGSKTFINHTQGIPDFFKQSFPEGFTWERVTTYEDGGVLTATQDTSLQDGCLIYNVKIRGVNFTSNGPVMQ
KKTLGWEAFTETLYPADGGLEGRNDMALKLVGGSHLIANIKTTYRSKKPAKNLKMPGVYYVDYRLERIKEANNET
YVEQHEVAVARY
Inactive control
TagGFP2_single_chain_noHis_asym_163-TagBFP
(SEQ ID NO: 50)
HMSGGEELFAGIVPVLIELDGDVHGHKFSVRGEGEGDADYGKLEIKFICTTGKLPVPWPTLVTTLCYGIQCFARY
PEHMKMNDFFKSAMPEGYIQERTIQFQDDGKYKTRGEVKFEGDTLVNRIELKGKDFKEDGNILGHKLEYSFNSHN
VYIRPDKANNGLEANFKTRHNIEGGGVQLADHYQTNVPLGDGPVLIPINHYLSTQTKISKDRNEARDHMVLLESF
SACCHTGGSGGSDELKYELEKSTRELQKSTDELEKSTEELERNPSKDVLVENNELIVRNNKIIVKNNIIIVRTEK
KGSGGSGDELKEELEKSTRELDKSTKKLERSTEELKRNPSKDALVENNKLIVENNTIIVRNNDIIVRTRKKGSGG
SGDELKEELEKSTRELKKSTKELQKSTEELERNPSKDALVKNNKLIADNNRIIVRNNTIIVRDIKASGGSGSELI
KENMHMKLYMEGTVDNHHFKCTSEGEGKPYEGTQTMRIKVVEGGPLPFAFDILATSFLYGSKTFINHTQGIPDFF
KQSFPEGFTWERVTTYEDGGVLTATQDTSLQDGCLIYNVKIRGVNFTSNGPVMQKKTLGWEAFTETLYPADGGLE
GRNDMALKLVGGSHLIANIKTTYRSKKPAKNLKMFGVYYVDYRLERIKEANNETYVEQHEVAVARY
Inactive control
TagGFP2-single_chain_noHis_asym_163-TagBFP
(SEQ ID NO: 51)
(MGSSHHHHHHSSGLVPRGS)HMSGGEELFAGIVPVLIELDGDVHGHKFSVRGEGEGDADYGRLEIKFICTTGKL
PVPWPTLVTTLCYGIQCFARYPEHMKMNDFFKSAMPEGYIQERTIQFQDDGKYKTRGEVKFEGDTLVNRIELKGK
DFKEDGNILGHKLEYSFNSHNVYIRPDKANNGLEANFKTRHNIEGGGVQLADHYQTNVPLGDGPVLIPINHYLST
QTKISKDRNEARDHMVLLESFSACCHTGGSGGSDELKYELEKSTRELQKSTDELEKSTEELERNPSKDVLVENNE
LIVRNNKIIVKNNIIIVRTEKKGSGGSGDELKEELEKSTRELDKSTKKLERSTEELKRNPSKDALVENNKLIVEN
NTIIVRNNDIIVRTRKKGSGGSGDELKEELEKSTRELKKSTKELQKSTEELERNPSKDALVKNNKLIADNNRIIV
RNNTIIVRDIKASGGSGSELIKENMHMKLYMEGTVDNHHFKCTSEGEGKPYEGTQTMRIKVVEGGPLPFAFDILA
TSFLYGSKTFINHTQGIPDFFKQSFPEGFTWERVTTYEDGGVLTATQDTSLQDGCLIYNVKIRGVNFTSNGPVMQ
KKTLGWEAFTETLYPADGGLEGRNDMALKLVGGSHLIANIKTTYRSKKPAKNLKMPGVYYVDYRLERIKEANNET
YVEQHEVAVARY
Inactive control
TagGFP2-single_chain_noHis_asym_163-TagBFP
(SEQ ID NO: 52)
HMSGGEELFAGIVPVLIELDGDVHGHKFSVRGEGEGDADYGKLEIKFICTTGKLPVPWPTLVTTLCYGIQCFARY
PEHMKMNDFFKSAMPEGYIQERTIQFQDDGKYKTRGEVKFEGDTLVNRIELKGKDFKEDGNILGHKLEYSFNSHN
VYIRPDKANNGLEANFKTRHNIEGGGVQLADHYQTNVPLGDGPVLIPINHYLSTQTKISKDRNEARDHMVLLESF
SACCHTGGSGGSDELKYELEKSTRELQKSTDELEKSTEELERNPSKDVLVENNELIVRNNKIIVKNNIIIVRTEK
KGSGGSGDELKEELEKSTRELDKSTKKLERSTEELKRNPSKDALVENNKLIVENNTIIVRNNDIIVRTRKKGSGG
SGDELKEELEKSTRELKKSTKELQKSTEELERNPSKDALVKNNKLIADNNRIIVRNNTIIVRDIKASGGSGSELI
KENMHMKLYMEGTVDNHHFKCTSEGEGKPYEGTQTMRIKVVEGGPLPFAFDILATSFLYGSKTFINHTQGIPDFF
KQSFPEGFTNERVTTYEDGGVLTATQDTSLQDGCLIYNVKIRGVNFTSNGPVMQKKTLGWEAFTETLYPADGGLE
GRNDMALKLVGGSHLIANIKTTYRSKKPAKNLKMPGVYYVDYRLERIKEANNETYVEQHEVAVARY
Inactive control
cpmoxCerulean_v2
(SEQ ID NO: 53)
(MGSSHHHHHHSSGENLY)FQGSGSGGIHGNVYITADKQKNGIKANFGLNSNVEDGSVQLADHYQQNTPIGDGPV
LLPDNHYLSTQSALSKDPNEKRDHMVLLEFVTAAGITLGMDELYKGGTGGSMVSKGEELFTGVVPILVELDGDVN
GHKFSVRGEGEGDATNGKLTLKFISTTGKLPVPWPTLVTTLSWGVQSFARYPDHMKQHDFFKSAMPEGYVQERTI
FFKDDGTYKTRAEVKFEGDTLVNRIELKGIDFKEDGNILGHKLEY*
Inactive
cpmoxCerulean_v2
(SEQ ID NO: 54)
FQGSGSGGIHGNVYITADKQKNGIKANFGLNSNVEDGSVQLADHYQQNTPIGDGPVLLPDNHYLSTQSALSKDPN
EKRDHMVLLEFVTAAGITLGMDELYKGGTGGSMVSKGEELFTGVVPILVELDGDVNGHKFSVRGEGEGDATNGKL
TLKFISTTGKLPVPWPTLVTTLSWGVQSFARYPDHMKQHDFFKSAMPEGYVQERTIFFKDDGTYKTRAEVKFEGD
TLVNRIELKGIDFKEDGNILGHKLEY*
Inactive
SB13(2 + 1)-cpmoxCerulean3_v2
(SEQ ID NO: 55)
(MGSSHHHHHHSSGENLY)FQGSGSGSTKYELRRALEELEKALRELKKSLDELERSLEELEKNPSEDALVENNRL
NVENNKIIVEVLRIIAEVLKINAKSDGSGSGSTKYELRRALEELEKALRELKKSLDELERSLEELEKNPSEDALV
ENNRLNVENNKIIVEVLRTIAEVLKINAKSDGSGIHGNVYITADKQKNGIKANFGLNSNVEDGSVQLADHYQQNT
PIGDGPVLLPDNHYLSTQSALSKDPNEKRDHMVLLEFVTAAGITLGMDELYKGGTGGSMVSKGEELFTGVVPILV
ELDGDVNGHKFSVRGEGEGDATNGKLTLKFISTTGKLPVPWPTLVTTLSWGVQSFARYPDHMKQHDFFKSAMPEG
YVQERTIFFKDDGTYKTRAEVKFEGDTLVNRIELKGIDFKEDGNILGHKLEYGGSTKYELRRALEELEKALRELK
KSLDELERSLEELEKNPSEDALVENNRLNVENNKIIVEVLRIIAEVLKINAKSD*
Inactive control
SB13(2 + 1)-cpmoxCerulean3_v2
(SEQ ID NO: 56)
FQGSGSGSTKYELRRALEELEKALRELKKSLDELERSLEELEKNPSEDALVENNRLNVENNKIIVEVLRIIAEVL
KINAKSDGSGSGSTKYELRRALEELEKALRELKKSLDELERSLEELEKNPSEDALVENNRLNVENNKIIVEVLRT
IAEVLKINAKSDGSGIHGNVYITADKQKNGIKANFGLNSNVEDGSVQLADHYQQNTPIGDGPVLLPDNHYLSTQS
ALSKDPNEKRDHMVLLEFVTAAGITLGMDELYKGGTGGSMVSKGEELFTGVVPILVELDGDVNGHKFSVRGEGEG
DATNGKLTLKFISTTGKLPVPWPTLVTTLSWGVQSFARYPDHMKQHDFFKSAMPEGYVQERTIFFKDDGTYKTRA
EVKFEGDTLVNRIELKGIDFKEDGNILGHKLEYGGSTKYELRRALEELEKALRELKKSLDELERSLEELEKNPSE
DALVENNRLNVENNKIIVEVLRIIAEVLKINAKSD*
Inactive control
SB13(2 + 1)-cpmoxCerulean3_v2-cfSGFP2
(SEQ ID NO: 77)
(MGSSHHHHHHSSGENLY)FQGSGSGSTKYELRRALEELEKALRELKKSLDELERSLEELEKNPSEDALVENNRL
NVENNKIIVEVLRIIAEVLKINAKSDGSGSGSTKYELRRALEELEKALRELKKSLDELERSLEELEKNPSEDALV
ENNRLNVENNKIIVEVLRIIAEVLKINAKSDGSGIHGNVYITADKQKNGIKANFGLNSNVEDGSVQLADHYQQNT
PIGDGPVLLPDNHYLSTQSALSKDPNEKRDHMVLLEFVTAAGITLGMDELYKGGTGGSMVSKGEELFTGVVPILV
ELDGDVNGHKFSVRGEGEGDATNGKLTLKFISTTGKLPVPWPTLVTTLSWGVQSFARYPDHMKQHDFFKSAMPEG
YVQERTIFFKDDGTYKTRAEVKFEGDTLVNRIELKGIDFKEDGNILGHKLEYGGSTKYELRRALEELEKALRELK
KSLDELERSLEELEKNPSEDALVENNRLNVENNKIIVEVLRIIAEVLKINAKSDMVSKGEELFTGVVPILVELDG
DVNGHKFSVSGEGEGDATYGKLTLKFISTTGKLPVPWPTLVTTLTYGVQMFARYPDHMKQHDFFKSAMPEGYVQE
RTIFFKDDGNYKTRAEVKFEGDTLVNRIELKGIDFKEDGNILGHKLEYNYNSHNVYITADKQKNGIKANFKIRHN
IEDGGVQLADHYQQNTPIGDGPVLLPDNHYLSTQSKLSKDPNEKRDHMVLLEFVTAAGITLGMDELYK
Inactive control
SB13(2 + 1)-cpmoxCerulean3_v2-cfSGFP2
(SEQ ID NO: 57)
FQGSGSGSTKYELRRALEELEKALRELKKSLDELERSLEELEKNPSEDALVENNRLNVENNKIIVEVLRIIAEVL
KINAKSDGSGSGSTKYELRRALEELEKALRELKKSLDELERSLEELEKNPSEDALVENNRLNVENNKIIVEVLRI
IAEVLKINAKSDGSGIHGNVYITADKQKNGIKANFGLNSNVEDGSVQLADHYQQNTPIGDGPVLLPDNHYLSTQS
ALSKDPNEKRDHMVLLEFVTAAGITLGMDELYKGGTGGSMVSKGEELFTGVVPILVELDGDVNGHKFSVRGEGEG
DATNGKLTLKFISTTGKLPVPWPTLVTTLSWGVQSFARYPDHMKQHDFFKSAMPEGYVQERTIFFKDDGTYKTRA
EVKFEGDTLVNRIELKGIDFKEDGNILGHKLEYGGSTKYELRRALEELEKALRELKKSLDELERSLEELEKNPSE
DALVENNRLNVENNKIIVEVLRIIAEVLKINAKSDMVSKGEELFTGVVPILVELDGDVNGHKFSVSGEGEGDATY
GKLTLKFISTTGKLPVPWPTLVTTLTYGVQMFARYPDHMKQHDFFKSAMPEGYVQERTIFFKDDGNYKTRAEVKF
EGDTLVNRIELKGIDFKEDGNILGHKLEYNYNSHNVYITADKQKNGIKANFKIRHNIEDGGVQLADHYQQNTPIG
DGPVLLPDNHYLSTQSKLSKDPNEKRDHMVLLEFVTAAGITLGMDELYK
Inactive control
SB13.2(2 + 1)-cpmoxCerulean3_v2-cfSGFP2
(SEQ ID NO: 58)
(MGSSHHHHHHSSGENLY)FQGSGSGSTKYELRRALEELEKALRELKKSLDELERSLEELEKNPSEDALVENNRL
NVENNKIIVEVLRIIAEVLKINAKSDGSGSGSTKYELRRALEELEKALRELKKSLDELERSLEELEKNPSEDALV
ENNRLNVENNKIIVEVLRIIAEVLKINAKEDGSGIHGNVYITADKQKNGIKANFGLNSNVEDGSVQLADHYQQNT
PIGDGPVLLPDNHYLSTQSALSKDPNEKRDHMVLLEFVTAAGITLGMDELYKGGTGGSMVSKGEELFTGVVPILV
ELDGDVNGHKFSVRGEGEGDATNGKLTLKFISTTGKLPVPWPTLVTTLSWGVQSFARYPDHMKQHDFFKSAMPEG
YVQERTIFFKDDGTYKTRAEVKFEGDTLVNRIELKGIDFKEDGNILGHKLEYGSGSTKYELRRALEELEKALREL
KKSLDELERSLEELEKNPSEDALVENNKIIVEVLRIIVEVLRIIAEVLKINAKSDMVSKGEELFTGVVPILVELD
GDVNGHKFSVSGEGEGDATYGKLTLKFISTTGKLPVPWPTLVTTLTYGVQMFARYPDHMKQHDFFKSAMPEGYVQ
ERTIFFKDDGNYKTRAEVKFEGDTLVNRIELKGIDFKEDGNILGHKLEYNYNSHNVYITADKQKNGIKANFKIRH
NIEDGGVQLADHYQQNTPIGDGPVLLPDNHYLSTQSKLSKDPNEKRDHMVLLEFVTAAGITLGMDELYK
Inactive control
SB13.2(2 + 1)-cpmoxCerulean3_v2-cfSGFP2
(SEQ ID NO: 59)
FQGSGSGSTKYELRRALEELEKALRELKKSLDELERSLEELEKNPSEDALVENNRLNVENNKIIVEVLRIIAEVL
KINAKSDGSGSGSTKYELRRALEELEKALRELKKSLDELERSLEELEKNPSEDALVENNRLNVENNKIIVEVLRI
IAEVLKINAKEDGSGIHGNVYITADKQKNGIKANFGLNSNVEDGSVQLADHYQQNTPIGDGPVLLPDNHYLSTQS
ALSKDPNEKRDHMVLLEFVTAAGITLGMDELYKGGTGGSMVSKGEELFTGVVPILVELDGDVNGHKFSVRGEGEG
DATNGKLTLKFISTTGKLPVPWPTLVTTLSWGVQSFARYPDHMKQHDFFKSAMPEGYVQERTIFFKDDGTYKTRA
EVKFEGDTLVNRIELKGIDFKEDGNILGHKLEYGSGSTKYELRRALEELEKALRELKKSLDELERSLEELEKNPS
EDALVENNKIIVEVLRIIVEVLRIIAEVLKINAKSDMVSKGEELFTGVVPILVELDGDVNGHKFSVSGEGEGDAT
YGKLTLKFISTTGKLPVPWPTLVTTLTYGVQMFARYPDHMKQHDFFKSAMPEGYVQERTIFFKDDGNYKTRAEVK
FEGDTLVNRIELKGIDFKEDGNILGHKLEYNYNSHNVYITADKQKNGIKANFKIRHNIEDGGVQLADHYQQNTPI
GDGPVLLPDNHYLSTQSKLSKDPNEKRDHMVLLEFVTAAGITLGMDELYK
Inactive control
163(2 + 1)-cpmoxCerulean3_v2
This embodiment shows pH-responsive fluorescence intensity modulation
due to fused helical bundle pH-responsive conformational switching that
is allosterically coupled to chromophore environment.
(SEQ ID NO: 60)
(MGSSHHHHHHSSGENLY)FQGSGSGSEALYELEKATRELKKATDELERATEELEKNPSEDALVEHNRLIAEHNK
IIAEHNRIIAKVLKGSGSGSEALYELEKATRELKKATDELERATEELEKNPSEDALVEHNRLIAEHNKIIAEHNR
IIAKVLKGSGIHGNVYITADKQKNGIKANFGLNSNVEDGSVQLADHYQQNTPIGDGPVLLPDNHYLSTQSALSKD
PNEKRDHMVLLEFVTAAGITLGMDELYKGGTGGSMVSKGEELFTGVVPILVELDGDVNGHKFSVRGEGEGDATNG
KLTLKFISTTGKLPVPWPTLVTTLSWGVQSFARYPDHMKQHDFFKSAMPEGYVQERTIFFKDDGTYKTRAEVKFE
GDTLVNRIELKGIDFKEDGNILGHKLEYGGSEALYELEKATRELKKATDELERATEELEKNPSEDALVEHNRLTA
EHNKIIAEHNRIIAKVLK
163(2 + 1)-cpmoxCerulean3_v2:
This embodiment shows pH-responsive fluorescence intensity modulation
due to fused helical bundle pH-responsive conformational switching that
is allosterically coupled to chromophore environment.
(SEQ ID NO: 61)
FQGSGSGSEALYELEKATRELKKATDELERATEELEKNPSEDALVEHNRLIAEHNKIIAEHNRIIAKVLKGSGSG
SEALYELEKATRELKKATDELERATEELEKNPSEDALVEHNRLIAEHNKIIAEHNRIIAKVLKGSGIHGNVYITA
DKQKNGIKANFGLNSNVEDGSVQLADHYQQNTPIGDGPVLLPDNHYLSTQSALSKDPNEKRDHMVLLEFVTAAGI
TLGMDELYKGGTGGSMVSKGEELFTGVVPILVELDGDVNGHKFSVRGEGEGDATNGKLTLKFISTTGKLPVPWPT
LVTTLSWGVQSFARYPDHMKQHDFFKSAMPEGYVQERTIFFKDDGTYKTRAEVKFEGDTLVNRIELKGIDFKEDG
NILGHKLEYGGSEALYELEKATRELKKATDELERATEELEKNPSEDALVEHNRLTAEHNKIIAEHNRIIAKVLK
163.2(2 + 1)-cpmoxCerulean3_v2:
This embodiment shows pH-responsive fluorescence intensity modulation
due to fused helical bundle pH-responsive conformational switching that
is allosterically coupled to chromophore environment.
(SEQ ID NO: 62)
(MGSSHHHHHHSSGENLY)FQGSGSGSEALYELEKATRELKKATDELERATEELEKNPSEDALVEHNRLIAEHNK
IIAEHNRIIAKVLKGSGSGSEALYELEKATRELKKATDELERATEELEKNPSEDALVEHNPLIAEHNKIIAEHNR
IIAKVLKGSGIHGNVYITADKQKNGIKANFGLNSNVEDGSVQLADHYQQNTPIGDGPVLLPDNHYLSTQSALSKD
PNEKRDHMVLLEFVTAAGITLGMDELYKGGTGGSMVSKGEELFTGVVPILVELDGDVNGHKFSVRGEGEGDATNG
KLTLKFISTTGKLPVPWPTLVTTLSWGVQSFARYPDHMKQHDFFKSAMPEGYVQERTIFFKDDGTYKTRAEVKFE
GDTLVNRIELKGIDFKEDGNILGHKLEYGSGSEALYELEKATRELKKATDELERATEELEKNPSEDALVEHNRLI
AEHNKIIAEHNRIIAKVLK
163.2(2 + 1)-cpmoxCerulean3_v2:
This embodiment shows pH-responsive fluorescence intensity modulation
due to fused helical bundle pH-responsive conformational switching that
is allosterically coupled to chromophore environment.
(SEQ ID NO: 63)
FQGSGSGSEALYELEKATRELKKATDELERATEELEKNPSEDALVEHNRLIAEHNKIIAEHNRIIAKVLKGSGSG
SEALYELEKATRELKKATDELERATEELEKNPSEDALVEHNPLIAEHNKIIAEHNRIIAKVLKGSGIHGNVYITA
DKQKNGIKANFGLNSNVEDGSVQLADHYQQNTPIGDGPVLLPDNHYLSTQSALSKDPNEKRDHMVLLEFVTAAGI
TLGMDELYKGGTGGSMVSKGEELFTGVVPILVELDGDVNGHKFSVRGEGEGDATNGKLTLKFISTTGKLPVPWPT
LVTTLSWGVQSFARYPDHMKQHDFFKSAMPEGYVQERTIFFKDDGTYKTRAEVKFEGDTLVNRIELKGIDFKEDG
NILGHKLEYGSGSEALYELEKATRELKKATDELERATEELEKNPSEDALVEHNRLIAEHNKIIAEHNRIIAKVLK
(I56V)163.2(2 + 1)-cpmoxCerulean3_v2:
This embodiment shows pH-responsive fluorescence intensity modulation
due to fused helical bundle pH-responsive conformational switching that
is allosterically coupled to chromophore environment.
(SEQ ID NO: 64)
(MGSSHHHHHHSSGENLY)FQGSGSGSEALYELEKATRELKKATDELERATEELEKNPSEDALVEHNRLIAEHNK
IVAEHNRIIAKVLKGSGSGSEALYELEKATRELKKATDELERATEELEKNPSEDALVEHNRLIAEHNKIVAEHNR
IIAKVLKGSGIHGNVYITADKQKNGIKANFGLNSNVEDGSVQLADHYQQNTPIGDGPVLLPDNHYLSTQSALSKD
PNEKRDHMVLLEFVTAAGITLGMDELYKGGTGGSMVSKGEELFTGVVPILVELDGDVNGHKFSVRGEGEGDATNG
KLTLKFISTTGKLPVPWPTLVTTLSWGVQSFARYPDHMKQHDFFKSAMPEGYVQERTIFFKDDGTYKTRAEVKFE
GDTLVNRIELKGIDFKEDGNILGHKLEYGSGSEALYELEKATRELKKATDELERATEELEKNPSEDALVEHNRLI
AEHNKIVAEHNRIIAKVLK
(I56V)163.2(2 + 1)-cpmoxCerulean3_v2:
This embodiment shows pH-responsive fluorescence intensity modulation
due to fused helical bundle pH-responsive conformational switching that
is allosterically coupled to chromophore environment.
(SEQ ID NO: 65)
FQGSGSGSEALYELEKATRELKKATDELERATEELEKNPSEDALVEHNRLIAEHNKIVAEHNRIIAKVLKGSGSG
SEALYELEKATRELKKATDELERATEELEKNPSEDALVEHNRLIAEHNKIVAEHNRIIAKVLKGSGIHGNVYITA
DKQKNGIKANFGLNSNVEDGSVQLADHYQQNTPIGDGPVLLPDNHYLSTQSALSKDPNEKRDHMVLLEFVTAAGI
TLGMDELYKGGTGGSMVSKGEELFTGVVPILVELDGDVNGHKFSVRGEGEGDATNGKLTLKFISTTGKLPVPWPT
LVTTLSWGVQSFARYPDHMKQHDFFKSAMPEGYVQERTIFFKDDGTYKTRAEVKFEGDTLVNRIELKGIDFKEDG
NILGHKLEYGSGSEALYELEKATRELKKATDELERATEELEKNPSEDALVEHNRLIAEHNKIVAEHNRIIAKVLK
163.2(2 + 1)-cpmoxCerulean3_v2-cfSGFP2:
This embodiment shows pH-responsive fluorescence intensity modulation
due to fused helical bundle pH-responsive conformational switching that
is allosterically coupled to chromophore environment.
(SEQ ID NO: 66)
MGSGSEALYELEKATRELKKATDELERATEELEKNPSEDALVEHNRLIAEHNKIIAEHNRIIAKVLKGSGSGSEA
LYELEKATRELKKATDELERATEELEKNPSEDALVEHNRLIAEHNKIIAEHNRIIAKVLKGSGIHGNVYITADKQ
KNGIKANFGLNSNVEDGSVQLADHYQQNTPIGDGPVLLPDNHYLSTQSALSKDPNEKRDHMVLLEFVTAAGITLG
MDELYKGGTGGSMVSKGEELFTGVVPILVELDGDVNGHKFSVRGEGEGDATNGKLTLKFISTTGKLPVPWPTLVT
TLSWGVQSFARYPDHMKQHDFFKSAMPEGYVQERTIFFKDDGTYKTRAEVKFEGDTLVNRIELKGIDFKEDGNIL
GHKLEYGSGSEALYELEKATRELKKATDELERATEELEKNPSEDALVEHNRLIAEHNKIIAEHNRIIAKVLKMVS
KGEELFTGVVPILVELDGDVNGHKFSVSGEGEGDATYGKLTLKFISTTGKLPVPWPTLVTTLTYGVQMFARYPDH
MKQHDFFKSAMPEGYVQERTIFFKDDGNYKTRAEVKFEGDTLVNRIELKGIDFKEDGNILGHKLEYNYNSHNVYI
TADKQKNGIKANFKIRHNIEDGGVQLADHYQQNTPIGDGPVLLPDNHYLSTQSKLSKDPNEKRDHMVLLEFVTAA
GITLGMDELYK
Control fusion of cpmoxCerulean3_v2 (a novel cpFP) and cfSGFP2
(SEQ ID NO: 67)
(MGSSHHHHHHSSGENLY)FQGSGSGIHGNVYITADKQKNGIKANFGLNSNVEDGSVQLADHYQQNTPIGDGPVL
LPDNHYLSTQSALSKDPNEKRDHMVLLEFVTAAGITLGMDELYKGGTGGSMVSKGEELFTGVVPILVELDGDVNG
HKFSVRGEGEGDATNGKLTLKFISTTGKLPVPWPTLVTTLSWGVQSFARYPDHMKQHDFFKSAMPEGYVQERTIF
FKDDGTYKTRAEVKFEGDTLVNRIELKGIDFKEDGNILGHKLEYGSGSGMVSKGEELFTGVVPILVELDGDVNGH
KFSVSGEGEGDATYGKLTLKFISTTGKLPVPWPTLVTTLTYGVQMFARYPDHMKQHDFFKSAMPEGYVQERTIFF
KDDGNYKTRAEVKFEGDTLVNRIELKGIDFKEDGNILGHKLEYNYNSHNVYITADKQKNGIKANFKIRHNIEDGG
VQLADHYQQNTPIGDGPVLLPDNHYLSTQSKLSKDPNEKRDHMVLLEFVTAAGITLGMDELYK
Inactive control
Control fusion of cpmoxCerulean3_v2 (a novel cpFP) and cfSGFP2
(SEQ ID NO: 68)
FQGSGSGIHGNVYITADKQKNGIKANFGLNSNVEDGSVQLADHYQQNTPIGDGPVLLPDNHYLSTQSALSKDPNE
KRDHMVLLEFVTAAGITLGMDELYKGGTGGSMVSKGEELFTGVVPILVELDGDVNGHKFSVRGEGEGDATNGKLT
LKFISTTGKLPVPWPTLVTTLSWGVQSFARYPDHMKQHDFFKSAMPEGYVQERTIFFKDDGTYKTRAEVKFEGDT
LVNRIELKGIDFKEDGNILGHKLEYGSGSGMVSKGEELFTGVVPILVELDGDVNGHKFSVSGEGEGDATYGKLTL
KFISTTGKLPVPWPTLVTTLTYGVQMFARYPDHMKQHDFFKSAMPEGYVQERTIFFKDDGNYKTRAEVKFEGDTL
VNRIELKGIDFKEDGNILGHKLEYNYNSHNVYITADKQKNGIKANFKIRHNIEDGGVQLADHYQQNTPIGDGPVL
LPDNHYLSTQSKLSKDPNEKRDHMVLLEFVTAAGITLGMDELYK
Inactive control
pH-reponsive cpFP pH sensor with optimized linker, with C-terminal
cfSGFP2. This embodiment shows pH-responsive fluorescence intensity
modulation due to fused helical bundle pH-responsive conformational
switching that is allosterically coupled to chromophore environment.
(SEQ ID NO: 69)
(MGSSHHHHHHSSGENLY)FQGSGSGDDEDIDRVLEELRRITEELDRITKDLERLTQELRRNPSVDALVKHNNAI
VRHNEIIVEHNRIILEVLELLLRSIGSGSGDREEIKKVLDELREATERLERATEELRRLTEELKKNPAVEVLVRH
NTIIVKHNKIIVDHNRIIVRVLELLEKTIGSGIHGNVYITADKQKNGIKANFGLNSNVEDGSVQLADHYQQNTPI
GDGPVLLPDNHYLSTQSALSKDPNEKRDHMVLLEFVTAAGITLGMDELYKGGTGGSMVSKGEELFTGVVPILVEL
DGDVNGHKFSVRGEGEGDATNGKLTLKFISTTGKLPVPWPTLVTTLSWGVQSFARYPDHMKQHDFFKSAMPEGYV
QERTIFFKDDGTYKTRAEVKFEGDTLVNRIELKGIDFKEDGNILGHKLEYGSGDKYEIRKVLKELKDITEELRNM
TKNLTDLTEELKRNPSVEILVKHNILIVEHNKIIVEHNRIIVDVLELIRKAIMVSKGEELFTGVVPILVELDGDV
NGHKFSVSGEGEGDATYGKLTLKFISTTGKLPVPWPTLVTTLTYGVQMFARYPDHMKQHDFFKSAMPEGYVQERT
IFFKDDGNYKTRAEVKFEGDTLVNRIELKGIDFKEDGNILGHKLEYNYNSHNVYITADKQKNGIKANFKIRHNIE
DGGVQLADHYQQNTPIGDGPVLLPDNHYLSTQSKLSKDPNEKRDHMVLLEFVTAAGITLGMDELYK
pH-reponsive cpFP pH sensor with optimized linker, with C-terminal
cfSGFP2. This embodiment shows pH-responsive fluorescence intensity
modulation due to fused helical bundle pH-responsive conformational
switching that is allosterically coupled to chromophore environment.
(SEQ ID NO: 70)
FQGSGSGDDEDIDRVLEELRRITEELDRITKDLERLTQELRRNPSVDALVKHNNAIVRHNEIIVEHNRIILEVLE
LLLRSIGSGSGDREEIKKVLDELREATERLERATEELRRLTEELKKNPAVEVLVRHNTIIVKHNKIIVDHNRIIV
RVLELLEKTIGSGIHGNVYITADKQKNGIKANFGLNSNVEDGSVQLADHYQQNTPIGDGPVLLPDNHYLSTQSAL
SKDPNEKRDHMVLLEFVTAAGITLGMDELYKGGTGGSMVSKGEELFTGVVPILVELDGDVNGHKFSVRGEGEGDA
TNGKLTLKFISTTGKLPVPWPTLVTTLSWGVQSFARYPDHMKQHDFFKSAMPEGYVQERTIFFKDDGTYKTRAEV
KFEGDTLVNRIELKGIDFKEDGNILGHKLEYGSGDKYEIRKVLKELKDITEELRNMTKNLTDLTEELKRNPSVEI
LVKHNILIVEHNKIIVEHNRIIVDVLELIRKAIMVSKGEELFTGVVPILVELDGDVNGHKFSVSGEGEGDATYGK
LTLKFISTTGKLPVPWPTLVTTLTYGVQMFARYPDHMKQHDFFKSAMPEGYVQERTIFFKDDGNYKTRAEVKFEG
DTLVNRIELKGIDFKEDGNILGHKLEYNYNSHNVYITADKQKNGIKANFKIRHNIEDGGVQLADHYQQNTPIGDG
PVLLPDNHYLSTQSKLSKDPNEKRDHMVLLEFVTAAGITLGMDELYK
pH-responsive cpFP pH sensor with optimized linker using heterodimer
ZCON133, with C-terminal cfSGFP2. This embodiment shows pH-responsive
fluorescence intensity modulation due to fused helical bundle pH-responsive
conformational switching that is allosterically coupled to chromophore
environment.
(SEQ ID NO: 71)
(MGSSHHHHHHSSGENLY)FQGSGSGSDKEYKLDRILRRLDELIKQLSRILEEIERLVDELEREPLDDKEVQDVT
ERIVELIDEHLELLKEYIKLLEEYIKTTKGSGTHGNVYITADKQKNGIKANFGLNSNVEDGSVQLADHYQQNTPI
GDGPVLLPDNHYLSTQSALSKDPNEKRDHMVLLEFVTAAGITLGMDELYKGGTGGSMVSKGEELFTGVVPILVEL
DGDVNGHKFSVRGEGEGDATNGKLTLKFISTTGKLPVPWPTLVTTLSWGVQSFARYPDHMKQHDFFKSAMPEGYV
QERTIFFKDDGTYKTRAEVKFEGDTLVNRIELKGIDFKEDGNILGHKLEYGSGSPSKEYQEKSAERQKELLHEYE
KLVRHLRELVEKLQRRELDKEEVLRRLVEILERLKDLHKKIEDAHRKNEEAHKENKMVSKGEELFTGVVPILVEL
DGDVNGHKFSVSGEGEGDATYGKLTLKFISTTGKLPVPWPTLVTTLTYGVQMFARYPDHMKQHDFFKSAMPEGYV
QERTIFFKDDGNYKTRAEVKFEGDTLVNRIELKGIDFKEDGNILGHKLEYNYNSHNVYITADKQKNGIKANFKIR
HNIEDGGVQLADHYQQNTPIGDGPVLLPDNHYLSTQSKLSKDPNEKRDHMVLLEFVTAAGITLGMDELYK
pH-responsive cpFP pH sensor with optimized linker using heterodimer
ZCON133, with C-terminal cfSGFP2. This embodiment shows pH-responsive
fluorescence intensity modulation due to fused helical bundle pH-responsive
conformational switching that is allosterically coupled to chromophore
environment.
(SEQ ID NO: 72)
FQGSGSGSDKEYKLDRILRRLDELIKQLSRILEEIERLVDELEREPLDDKEVQDVTERIVELIDEHLELLKEYIK
LLEEYIKTTKGSGTHGNVYITADKQKNGIKANFGLNSNVEDGSVQLADHYQQNTPIGDGPVLLPDNHYLSTQSAL
SKDPNEKRDHMVLLEFVTAAGITLGMDELYKGGTGGSMVSKGEELFTGVVPILVELDGDVNGHKFSVRGEGEGDA
TNGKLTLKFISTTGKLPVPWPTLVTTLSWGVQSFARYPDHMKQHDFFKSAMPEGYVQERTIFFKDDGTYKTRAEV
KFEGDTLVNRIELKGIDFKEDGNILGHKLEYGSGSPSKEYQEKSAERQKELLHEYEKLVRHLRELVEKLQRRELD
KEEVLRRLVEILERLKDLHKKIEDAHRKNEEAHKENKMVSKGEELFTGVVPILVELDGDVNGHKFSVSGEGEGDA
TYGKLTLKFISTTGKLPVPWPTLVTTLTYGVQMFARYPDHMKQHDFFKSAMPEGYVQERTIFFKDDGNYKTRAEV
KFEGDTLVNRIELKGIDFKEDGNILGHKLEYNYNSHNVYITADKQKNGIKANFKIRHNIEDGGVQLADHYQQNTP
IGDGPVLLPDNHYLSTQSKLSKDPNEKRDHMVLLEFVTAAGITLGMDELYK
pH-responsive cpFP pH sensor with optimized linker using heterodimer
ZCON133 with subunits in reverse order in primary sequence, with C-terminal
cfSGFP2. This embodiment shows pH-responsive fluorescence intensity
modulation due to fused helical bundle pH-responsive conformational
switching that is allosterically coupled to chromophore environment.
(SEQ ID NO: 73)
(MGSSHHHHHHSSGENLY)FQGSGSGSPSKEYQEKSAERQKELLHEYEKLVRHLRELVEKLQRRELDKEEVLRRL
VEILERLKDLHKKIEDAHRKNEEAHKENKGSGIHGNVYITADKQKNGIKANFGLNSNVEDGSVQLADHYQQNTPI
GDGPVLLPDNHYLSTQSALSKDPNEKRDHMVLLEFVTAAGITLGMDELYKGGTGGSMVSKGEELFTGVVPILVEL
DGDVNGHKFSVRGEGEGDATNGKLTLKFISTTGKLPVPWPTLVTTLSWGVQSFARYPDHMKQHDFFKSAMPEGYV
QERTIFFKDDGTYKTRAEVKFEGDTLVNRIELKGIDFKEDGNILGHKLEYGSGSDKEYKLDRILRRLDELIKQLS
RILEEIERLVDELEREPLDDKEVQDVIERIVELIDEHLELLKEYIKLLEEYIKTTKMVSKGEELFTGVVPILVEL
DGDVNGHKFSVSGEGEGDATYGKLTLKFISTTGKLPVPWPTLVTTLTYGVQMFARYPDHMKQHDFFKSAMPEGYV
QERTIFFKDDGNYKTRAEVKFEGDTLVNRIELKGIDFKEDGNILGHKLEYNYNSHNVYITADKQKNGIKANFKIR
HNIEDGGVQLADHYQQNTPIGDGPVLLPDNHYLSTQSKLSKDPNEKRDHMVLLEFVTAAGITLGMDELYK
pH-responsive cpFP pH sensor with optimized linker heterodimer
ZCON133 with subunits in reverse order in primary sequence, with C-terminal
cfSGFP2. This embodiment shows pH-responsive fluorescence intensity
modulation due to fused helical bundle pH-responsive conformational
switching that is allosterically coupled to chromophore environment.
(SEQ ID NO: 74)
FQGSGSGSPSKEYQEKSAERQKELLHEYEKLVRHLRELVEKLQRRELDKEEVLRRLVEILERLKDLHKKIEDAHR
KNEEAHKENKGSGIHGNVYITADKQKNGIKANFGLNSNVEDGSVQLADHYQQNTPIGDGPVLLPDNHYLSTQSAL
SKDPNEKRDHMVLLEFVTAAGITLGMDELYKGGTGGSMVSKGEELFTGVVPILVELDGDVNGHKFSVRGEGEGDA
TNGKLTLKFISTTGKLPVPWPTLVTTLSWGVQSFARYPDHMKQHDFFKSAMPEGYVQERTIFFKDDGTYKTRAEV
KFEGDTLVNRIELKGIDFKEDGNILGHKLEYGSGSDKEYKLDRILRRLDELIKQLSRILEEIERLVDELEREPLD
DKEVQDVIERIVELIDEHLELLKEYIKLLEEYIKTTKMVSKGEELFTGVVPILVELDGDVNGHKFSVSGEGEGDA
TYGKLTLKFISTTGKLPVPWPTLVTTLTYGVQMFARYPDHMKQHDFFKSAMPEGYVQERTIFFKDDGNYKTRAEV
KFEGDTLVNRIELKGIDFKEDGNILGHKLEYNYNSHNVYITADKQKNGIKANFKIRHNIEDGGVQLADHYQQNTP
IGDGPVLLPDNHYLSTQSKLSKDPNEKRDHMVLLEFVTAAGITLGMDELYK

In one embodiment, the polypeptide includes changes to the highlighted residues (i.e., residues involved in hydrogen-bind networks) in Table 1, 2, or 3 of the polypeptides of 1-36 only to other polar amino acids.

In another embodiment, the polypeptide includes no changes to the highlighted residues of the polypeptides of SEQ ID NOs:1-36. In a further embodiment, all amino acid substitutions relative to the amino acid sequence of SEQ ID NOs: 1-40, 45-46, 60-66, 69-76, and 81-86 are conservative amino acid substitutions. In various embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

Alternatively, naturally occurring residues can be divided into groups based on common sidechain properties: (1) hydrophobic: Norleucine, Met, Ala, Val, Leo, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into G; Ile into Leu or into Val; Leu into Ile or into Val; Lys into Arg, into Gln or into Glu; Met into Leu, into Tyr or into Ile; Phe into Met, into Lea or into Tyr, Ser into Thr; Thr into Ser; Trp into Tyr; Tyr into Trp; and/or Phe into Val, into Ile or into Leu.

In another aspect, the disclosure provides non-naturally occurring polypeptide, comprising the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to the amino acid sequence selected from the group consisting of SEQ ID NOS:1-77 and 81-86. In one embodiment, the polypeptide includes changes to the highlighted residues in Table 1, 2, or 3 of the amino acid sequence selected from the group consisting of SEQ ID NOS:1-36 only to other polar amino acids. In a further embodiment, the polypeptide includes no changes to the highlighted residues in Table 1, 2, or 3 of the amino acid sequence selected from the group consisting of SEQ ID NOS:1-36. In a further embodiment, all amino acid substitutions relative to the amino acid sequence selected from the group consisting of SEQ ID NOS:1-77 and 81-86 are conservative amino acid substitutions.

In another embodiment, the disclosure comprises oligomeric polypeptide comprising two or more polypeptides of any embodiment or combination of embodiments disclosed herein. In one embodiment, the oligomeric polypeptides comprise a hetero-oligomer. The hetero-oligomer may be any suitable hetero-oligomer, including but not limited to heterodimers. Exemplary heterodimers provided herein include heterodimers between polypeptides comprises the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to:

(a) the amino acid sequence of SEQ ID NO:81 and the amino acid sequence of SEQ ID NO:82 (pro4);

(b) the amino acid sequence of SEQ ID NO:81 and the amino acid sequence of SEQ ID NO:84 (pro4);

(c) the amino acid sequence of SEQ ID NO:83 and the amino acid sequence of SEQ ID NO:82 (pro4);

(d) the amino acid sequence of SEQ ID NO:83 and the amino acid sequence of SEQ ID NO:84 (pro4); or

(e) the amino acid sequence of SEQ ID NO:85 and the amino acid sequence of SEQ ID NO:86 (pro5).

In another embodiment, the oligomeric polypeptides comprise a homo-oligomer. The homo-oligomer may be any suitable homo-oligomer, including but not limited to homotrimers. Exemplary heterodimers provided herein include homotrimers of the polypeptide comprising the amino acid sequence at least 25%, 30%, 35%, 40%, 45%, 50%, 55%, 60%, 65%, 70%, 75%, 80%, 83%, 90%, 95%, 96%, 97%, 98%, 99%, or 100% identical to a pRO-1 polypeptide (SEQ ID NOs:13-14), a prO-2 polypeptides (SEQ ID NOs: 1-12, 15-22, and 33-36), or a pRO-3 polypeptide (SEQ ID NOs:23-26).

The polypeptides of the disclosure may include additional residues at the N-terminus, C-terminus, internal to the polypeptide, or a combination thereof; these additional residues are not included in determining the percent identity of the polypeptides of the invention relative to the reference polypeptide. Such residues may be any residues suitable for an intended use, including but not limited to detectable proteins or fragments thereof (also referred to as “tags”). As used herein, “tags” include general detectable moieties (i.e.: fluorescent proteins, antibody epitope tags, etc.), therapeutic agents, purification tags (His tags, etc.), linkers, ligands suitable for purposes of purification, ligands to drive localization of the polypeptide, peptide domains that add functionality to the polypeptides, etc. Examples are provided herein.

For example, by fusing the polypeptide to a fluorescent protein, we are coupling the conformational change due to protonation of the buried histidines in the hydrogen bond networks at the interface of the helical bundle to conformational changes in the chromophore environment of the fused fluorescent protein. This provides a fluorescent readout of the conformation change. As will be understood by those of skill in the art, other functional subunits could be used in a similar manner to link the pH-based conformational change with a readout based on the function of the functional subunit.

As used throughout the present application, the term “polypeptide”, “peptide” and “protein” are used interchangeably in their broadest sense to refer to a sequence of subunit amino acids of any length, which can include genetically coded and non-genetically coded amino acids, chemically or biochemically modified or derivatized amino acids, and polypeptides having modified peptide backbones. The polypeptides of the invention may comprise L-amino acids+glycine, D-amino acids+glycine (which am resistant to L-amino acid-specific proteases in vivo), or a combination of D- and L-amino acids+glycine. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent n is understood by those of skill in the art.

In another aspect, the disclosure provides nucleic acids encoding the polypeptide of any embodiment or combination of embodiments of each aspect disclosed herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure we nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the expression vectors disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection. A method of producing a polypeptide according to the disclosure is an additional part of the disclosure. In one embodiment, the method comprises the steps of (a) culturing a host according to this aspect of the disclosure under conditions conducive to the expression of the polypeptide, and (b) optionally, recovering the expressed polypeptide. The expressed polypeptide can be recovered from the cell free extract or recovered from the culture medium. In another embodiment, the method comprises chemically synthesizing the polypeptides.

In another aspect, the disclosure provides methods for use of the polypeptides or the oligomeric polypeptides of any embodiment or combination of embodiments of the disclosure, for any suitable purpose, including but not limited to delivery of biologics into the cytoplasm through endosomal escape. Delivery methods relying on cell penetrating peptides, supercharged proteins, and lipid-fusing chemical reagents can be toxic because of nonspecific interactions with many types of membranes in a pH-independent manner. Thus, the disclosed polypeptides and oligomeric polypeptides provide a significant improvement over currently available tools.

In another aspect, the disclosure provides methods for designing the polypeptides or the oligomeric polypeptide of any embodiment or combination of embodiments of the disclosure, comprising a method as described in the examples that follow.

### Examples

Abstract:

The ability of naturally occurring proteins to change conformation in response to environmental changes is critical to biological function. The design of conformational switches remains a major challenge. Here we present a general strategy to design pH-responsive protein conformational switches by precisely pre-organizing histidine residues in buried hydrogen bond networks. We design homotrimers and heterodimers that are stable above pH 6.5, but undergo cooperative, large-scale conformational transitions when the pH is lowered and electrostatic and steric repulsion builds up as the network histidines become protonated. The pH range at which disassembly occurs, as well as the cooperativity of the transition, can be programmed by balancing the number of histidine-containing networks and the strength of the surrounding hydrophobic interactions. Upon disassembly, the designed proteins disrupt lipid membranes both in vitro and in vivo after being endocytosed in mammalian cells; the extent of disruption and the pH-dependence of membrane activity can be tuned such that no membrane activity is observed at pH 7 and substantial membrane activity is observed at and below pH 6. Our results are dynamic de novo proteins with switchable, conformation-dependent functions that provide a new route to addressing the endosomal escape challenge for intracellular delivery.

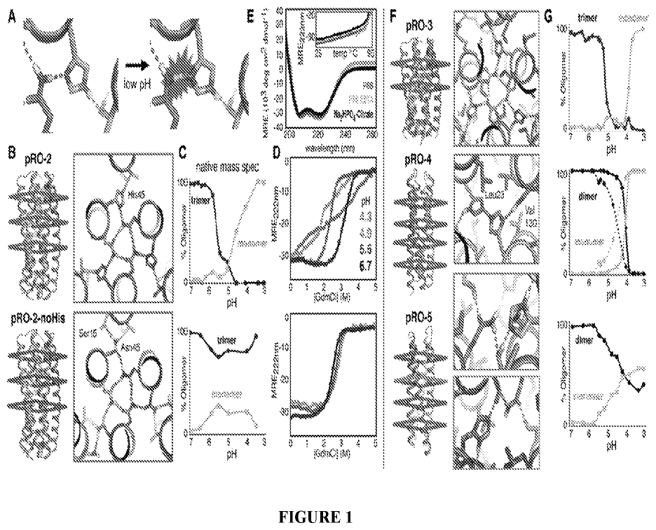

We explored the de novo design of protein systems undergoing pH-dependent conformation changes both because the subtlety of the protonation slate changes makes pH-dependence an excellent model problem and a challenging test of our understanding of protein energetics, and because programmable pH-induced conformational changes could have applications for engineering pH-dependent materials and intracellular delivery agents of biological cargo. We set out to create tunable pH-responsive oligomers (pRO's) by de novo designing parametric helical bundles with extensive histidine-containing networks in which the histidine N_{ε} and N_{δ} atoms are each making hydrogen bonds (FIG. 1). We hypothesized that designing networks with histidine residues that hydrogen bond across the oligomeric interface would result in disassembly at low pH because histidine side chain protonation would disrupt the hydrogen bond network, energetically destabilizing the assembled protein because of both the resultant steric and electrostatic repulsion and buried polar atoms that are unable to make hydrogen bonds (FIG. 1A). The repeating geometric cross-sections of parametric helical bundles allows hydrogen bond networks to be added or subtracted in a modular fashion, and we hypothesized that the pH range of disassembly, as well as the cooperativity, could be tuned by varying the number of histidine networks relative to the surrounding hydrophobic contacts.

We used a three-step procedure to computationally design helical bundles with extensive histidine-containing hydrogen bond networks that span inter-helical interfaces. First, oligomeric protein backbones with an inner and outer ring of α-helices were produced by systematically varying helical parameters using the Crick generating equations. Each inner helix was connected to an outer helix through a short designed loop to produce helix-turn-helix monomer subunits. Second, the HBNet™ method in Rosetta™ was extended to computationally design networks with buried histidine residues that accept a hydrogen bond across the oligomeric interface, and then used to select the very small fraction of backbones that accommodate multiple histidine networks (see Computational Design Methods). Third, the sequence of the rest of the protein (surface residues and the hydrophobic contacts surrounding the networks) was improved while keeping the histidine networks constrained. Synthetic genes encoding five parent designs (named pRO-1 to pRO-S) with multiple histidine-containing hydrogen bond networks and tight, complementary hydrophobic packing around the networks, along with variants (named pRO-2.1, pRO-2.2, etc.) were constructed (table 3).

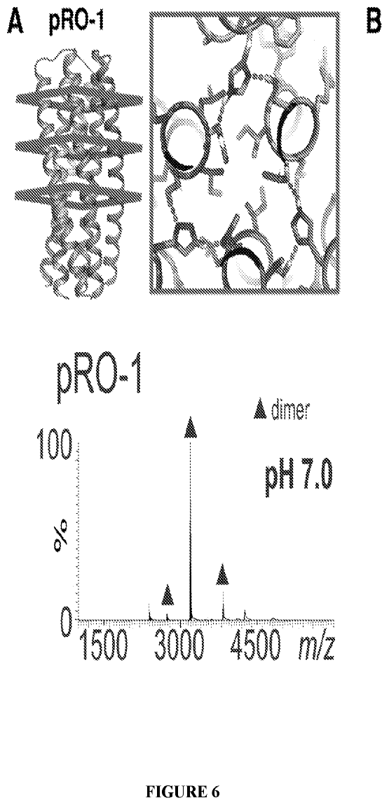

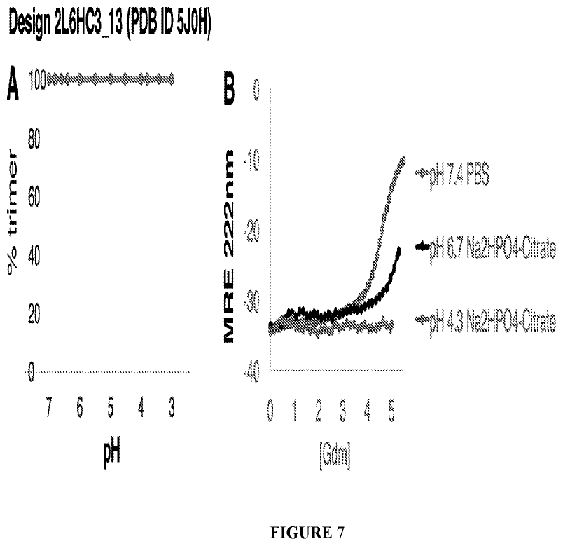

All of the designed proteins were well-expressed, soluble, and readily purified by Ni-NTA affinity chromatography, hexahistidine tag cleavage, and a second Ni-NTA step followed by gel filtration. Oligomeric state was assessed by size-exclusion chromatography (SEC) and native mass spectrometry (24). All parent designs assembled to the intended oligomeric state at pH 7 (FIG. 1) except for homotrimer design pRO-1, which appeared to be trimeric at high concentration by SEC but was primarily dimeric by native mass spectrometry at lower concentrations (FIG. 6); pRO-1 contains smaller, disjoint networks, each with a single histidine, whereas the successful parent designs all have highly-connected hydrogen bond networks that span across all helices of the bundle cross section. To assess the effectiveness of the design strategy, we used native mass spectrometry to study the effect of pH on oligomerization state(25, 26), evaluating each protein from pH 7 down to pH 3 (see Experimental Methods); designs pRO-2 through pRO-5 all exhibited pH-induced loss of the initial oligomeric state (FIG. 1). As a control, we subjected a previous design (2L6HC3_13(18); PDB ID 5J0H) with a structure similar to pRO-2 but lacking buried histidines to the same assays: changing buffer pH from 7 to as low as pH 3 resulted in no change in oligomeric state (FIG. 7A) or stability (FIG. 78). Design pRO-2 was chosen for further characterization, as it exhibited pH-induced disassembly between pH 5 and 6, which is within the range of endosomal pH(27, 28).

The pH-Dependent Conformational Switching is Due to the Designed Histidine Networks

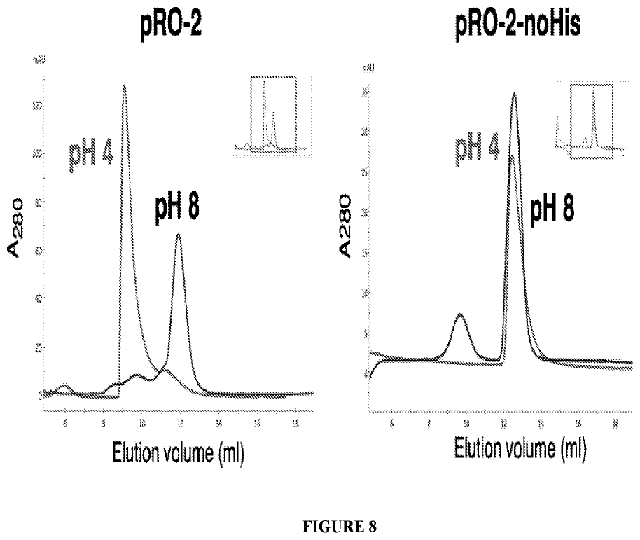

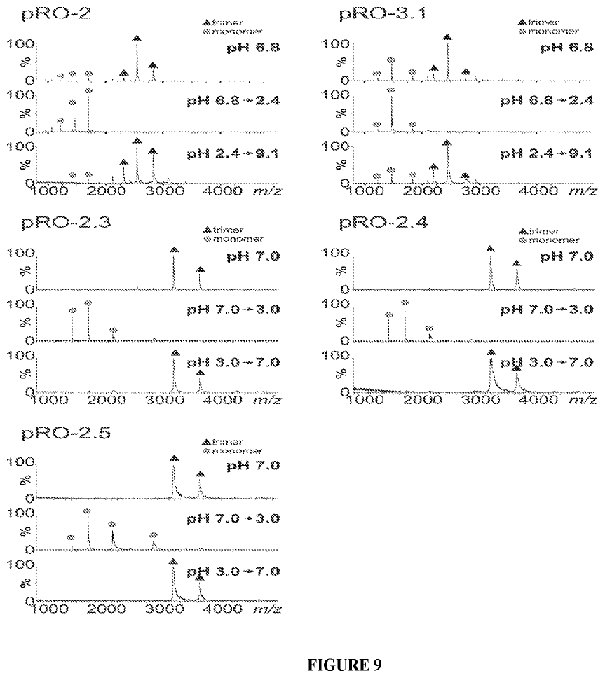

To specifically evaluate the role of the histidine networks in the pH-induced transition of pRO-2, we sought to design a variant that lacked the histidine residues but was otherwise identical in sequence. Mutating all histidine residues to asparagine resulted in poor soluble expression and aggregation, likely because the buried asparagine residues are unable to participate in hydrogen bonds; using HBNet™, we rescued the histidine to asparagine mutations by generating networks in which all buried polar atoms participate in hydrogen bonds (FIG. 1B, blue cross-sections). This new design (pRO-2-noHis), which differs by only six amino acids in each monomeric subunit, is well-behaved in solution and assembled to the intended trimeric state, but unlike pRO-2, remained trimeric at low pH (FIG. 1C and FIG. 8). Circular dichroism (CD) experiments showed that both proteins were helical and well-folded, and chemical denaturation by guanidinium chloride (GdmCl) showed that pRO-2 has decreased folding stability at low pH, whereas pRO-2-noHis stability was unaffected by change in pH (FIG. 1D). The histidines of pRO-2 do not participate in unintended metal interactions that contribute to assembly/disassembly, as addition of 10 mM EDTA had no effect on the helical fold or thermostability of design pRO-2 (FIG. 1E). Collectively, these results indicate that the observed pH-response is due to the designed histidine networks.

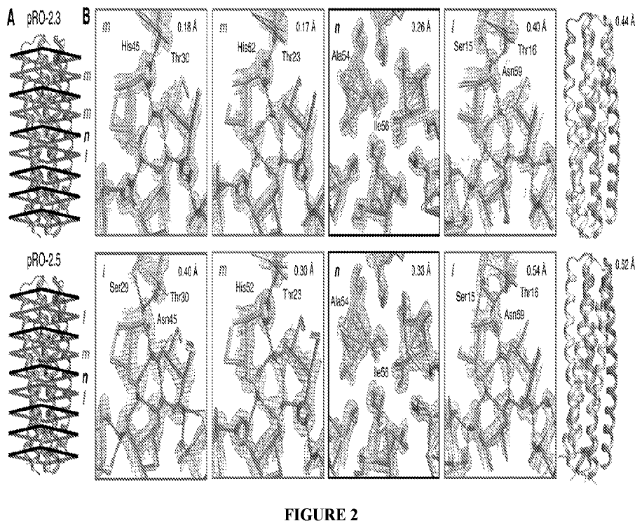

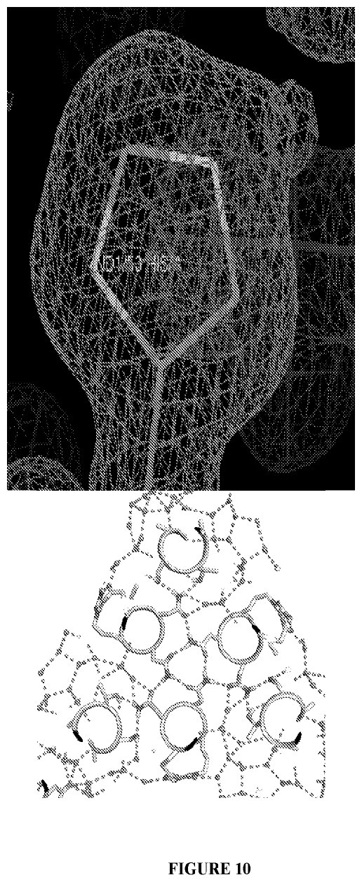

We set out to structurally characterize these designs, but both pRO-2 and pRO-2-noHis were resistant to crystallization efforts. To both test the modularity of our design strategy, as well as to generate additional constructs for crystallization, designs were made that combined networks from each of pRO-2 and pRO-2-noHis (Table 3). These variants remained soluble after disassembling and reassembled to their designed oligomeric state upon subsequent increase back to pH 7 (FIG. 9). Designs pRO-2.3 and pRO-2.5 (FIG. 2A) readily crystallized and X-ray crystal structures were determined at 1.28 Å and 1.55 Å resolution, respectively (FIG. 2B, FIG. 10, and Table 4). Design pRO-2.3, which differs from parent design pRO-2 by only two amino acids in each subunit, contains two histidine networks (red cross-sections) and one non-histidine network (blue cross-section); design pRO-2.5 differs from pRO-2 by five amino acids in each subunit and contains one histidine network and two non-histidine networks. In all cases, the hydrogen bond networks were nearly identical between the experimentally determined structures and the design models (FIG. 2). The ability to swap different types and placements of hydrogen bond networks at each layer without sacrificing structural accuracy highlights the modularity of our design strategy.

### Tuning of pH Set Point and Cooperativity

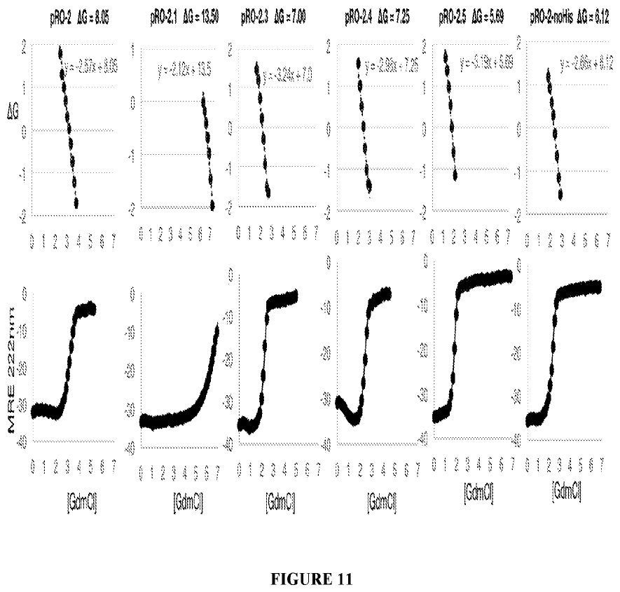

We take advantage of this modularity to systematically tune the pH response by developing a model of the pH-dependence of the free energy of assembly for a homotrimer with n pH-independent hydrophobic layers, m pH-dependent hydrogen bond network layers each containing three histidine residues, and l hydrogen bond network layers lacking histidine. We assume that the protonation of individual histidine residues within a network layer is cooperative—this is plausible since the protonation of one histidine residue will likely destabilize its surrounding interface, making the remaining histidine residues more accessible and substantially reducing the free energy cost of protonation. The pH-dependence of homotrimer assembly for such a system is then

$$\% trimer = \frac{100}{1 + {e}^{- \frac{1}{RT} [ \begin{matrix}n \cdot Δ {G}_{hydrophobic} + m \cdot Δ {G}_{polar\_m} \\ + l \cdot Δ {G}_{polar\_l} - 3 \cdot m \cdot ln ( 10 ) RT ( {pKa}_{His} - pH )\end{matrix} ]}} \qquad Eq . 1$$

where ΔG_{hydrophobic}, ΔG_{polar_m}, and ΔG_{polar_l} are the free energies of formation of hydrophobic layers, pH-responsive polar layers, and pH-independent polar layers respectively; R is the gas constant, and pKa_{His} (the pKa of solvent-exposed histidine) is taken to be 6.0. Equation I requires estimates of ΔG_{hydrophobic}, ΔG_{polar_m}, and ΔG_{polar_l}, which we obtained from guanidine denaturation experiments (FIG. 3B and FIG. 11). In this model, increases in n shift the pH of disassembly to lower pH values without affecting cooperativity (FIG. 3C top), and varying m while n and (m+l) are kept constant changes the cooperativity (steepness) of the transition without as large of an effect on the midpoint (FIG. 3C bottom).

To test the tuning of the pH-dependence of disassembly, we generated additional designs based on pRO-2 with different values of m, n and l by swapping one or two of the histidine networks (red cross-sections) for either hydrophobic-only interactions (black cross-sections) or the equivalent hydrogen bond network lacking histidine (blue cross-sections) in different combinations (FIG. 3A). These new designs were assessed by native mass spectrometry and found to assemble to the intended trimeric state at pH 7 and disassemble at a range of pH values (FIG. 3D). Because of the context-dependent effects discussed below, we did not directly fit these data to Eq. 1; instead the cooperativity of the transition (k) and the pH set point (pH0) were assessed by fitting the experimental data to a simple sigmoid model that assumes that the starting point is 100% trimer and the endpoint is 0% trimer:

$$\% trimer = \frac{100}{1 + {e}^{- k \cdot ( pH - pHo )}} \qquad Eq . 2$$

We compare the observed dependence of k and pH0 on m, n and l with the predictions of the model (Eq. 1) in the following sections.

### Tuning pH Set Point (FIG. 3C-D Top)

In Equation 1, the pH set point (pH0) is the pH at which the free energy of assembly (the quantity in square brackets) is zero. Designs with histidine networks replaced by hydrophobic layers have higher stability as assessed by chemical denaturation experiments (FIG. 3B); thus as expected, ΔG_{hydrophobic} is greater than ΔG_{polar_m}. The free energy of assembly at the pKa of histidine is given by the sum of the first three terms, and since ΔG_{hydrophobic} is greater than ΔG_{polar_m}, this sum can be increased by increasing the number of hydrophobic layers and reducing the number of histidine layers. The larger the sum, the greater the pH change required for the net free energy of assembly to be zero—hence pH0 can be lowered by increasing n (the number of hydrophobic layers) and/or reducing m (the number of histidine networks). Consistent with this prediction, replacing a single histidine network with a hydrophobic network (design pRO-2.1, purple curves) shifts the transition pH from above 5 down to ˜3.5, and replacing two histidine networks with hydrophobic networks (design pRO-2.2, pink curves) eliminates the pH response altogether (FIG. 3D top). Designs pRO-3 (red curves) and pRO-3.1 (orange curves) have two fewer total layers than pRO-2 and also behave as predicted: replacing a single histidine network layer with hydrophobics in these shorter designs increases the pH set point (FIG. 3D top). The Equation 1 model holds over the full set of designs tested: the larger the ratio of m to n, the higher the transition pH (FIG. 3E).

### Tuning Cooperativity (FIG. 3C-D Bottom)

In Equation 1, the transition cooperativity (k) is simply 3m, and replacing the histidine networks (m) with polar networks lacking histidines (l) with roughly equal contribution to stability at the pKa of histidine (ΔG_{polar_m} roughly equal to ΔG_{polar_l}) allows for tuning of the cooperativity of disassembly with little effect on stability (FIGS. 3B and 3C). At 5 μM trimer (FIG. 3D, bottom right panel), the cooperativity decreases through the series (m=3, l=0)(black) through (m=2, l=1)(cyan) to (m=1, l=2) (green), consistent with the model. Indeed, design pRO-2.5 (green curves), which has only one histidine network, is the least cooperative design tested and disassembles at approximately pH 4 (FIG. 3D bottom), despite having the lowest stability in chemical denaturation experiments (FIG. 3B).

### Context-Dependence

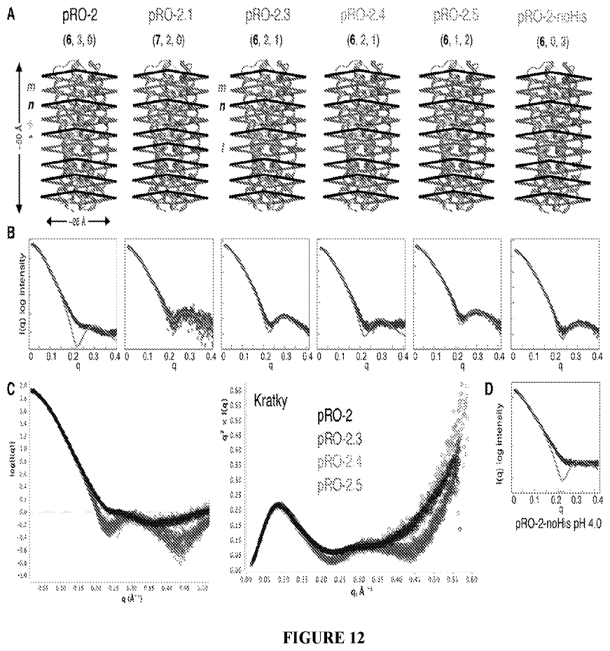

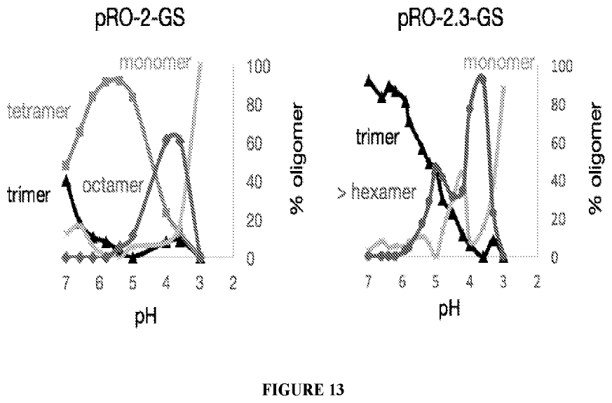

While Equation 1 qualitatively accounts for the dependence of disassembly and cooperativity on m, n and l, the location of the histidine network layers also contributes. For example, pRO-2.3 and pRO-2.4 have identical layer compositions (FIG. 3A) and nearly identical sequence compositions (Table 3), but pRO-2.4 disassembles at a higher transition pH and is less cooperative (FIG. 3D). Overall, designs with a histidine network close to the termini have higher transition pH values and less cooperative transitions. Histidine residues close to the termini are likely more accessible and hence easier to protonate, and this dynamic accessibility could better accommodate the destabilizing effect of protonation. Consistent with this hypothesis, designs pRO-2 and pRO-2.4, which have histidine networks closer to the termini, have higher flexibility as assessed by small-angle X-ray scattering (SAXS) measurements (29, 30) compared to designs pRO-2.1, pRO-2.3, pRO-2.5, and pRO-2-noHis, which do not have histidine networks close to the termini (FIG. 12 and Table 5); a correlation between flexibility and reduced cooperativity is also observed when the ordered helix-connecting loops are replaced by a flexible GS-linker (FIG. 13). Designs with histidine networks further away from the termini (and closer to the loop in the helical hairpin subunit) are presumably harder to initially protonate, but once protonated have a greater destabilizing effect that increases the accessibility of the other histidine positions, resulting in a more cooperative transition.

pH-Dependent Membrane Disruption

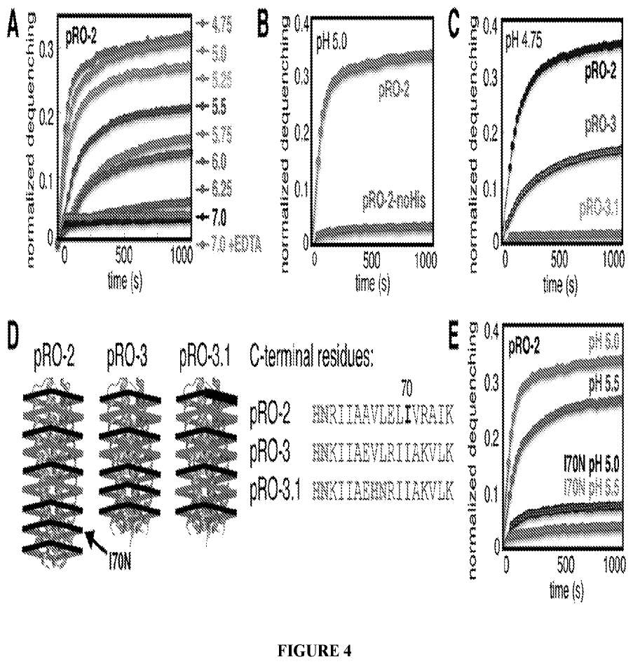

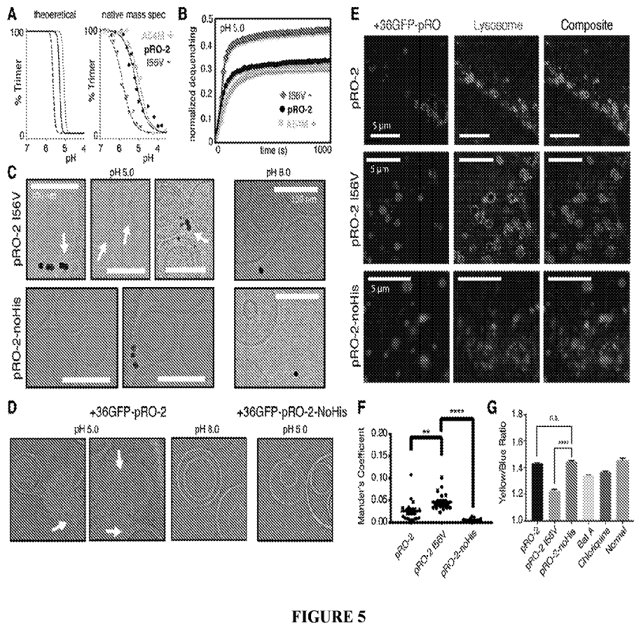

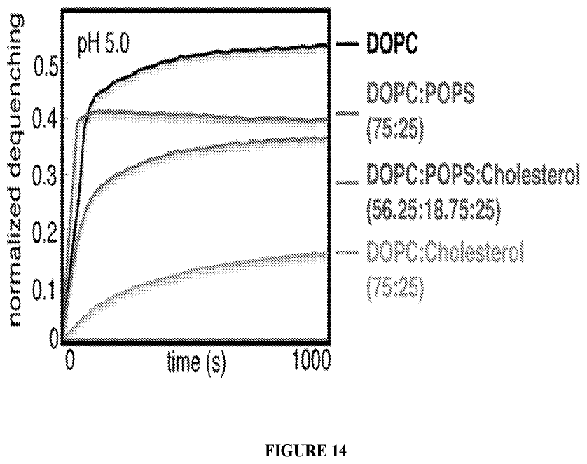

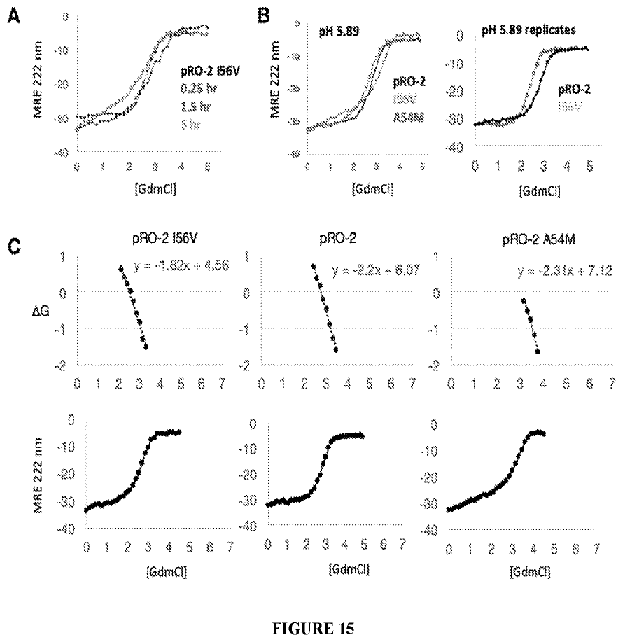

The trimer interface contains a number of hydrophobic residues that become exposed upon pH-induced disassembly; because amphipathic helices can disrupt membranes (17, 31), we investigated whether the designed proteins exhibit pH-dependent interactions with membranes. Purified protein with hexahistidine tag removed was added to synthetic liposomes containing the pH-insensitive fluorescent dye sulforhodamine B (SRB) at self-quenching concentrations over a range of pH values; leakage of liposome contents following disruption of the lipid membrane can be monitored through dequenching of the dye (32). Design pRO-2 caused pH-dependent liposome disruption at pH values as high as 6, with maximal activity around pH 5 (FIG. 4A). Design pRO-2-noHis which did not disassemble at low pH (FIG. 1C-D), showed no liposome activity at pH 5 (FIG. 4B). Design pRO-2 also caused pH-dependent disruption of liposomes with more native-like lipid compositions, although increased cholesterol resulted in decreased activity (FIG. 14). Design pRO-3 also caused pH-dependent liposome disruption (FIG. 4C); however, design pRO-3.1, which is even more pH-sensitive than design pRO-3 (FIG. 3D), did not exhibit any liposome disruption (FIG. 4C). The one major difference between pRO-3.1 compared to pRO-3 and pRO-2 is the lack of a contiguous stretch of hydrophobic amino acids at the C-terminus (FIG. 4D). These putative membrane-interacting residues are sequestered in the designed oligomeric state but likely exposed after pH-induced disassembly. To test this hypothesis, a central isoleucine in this region of pRO-2 was mutated to asparagine (I70N), which resulted in attenuation of pH-induced liposome disruption (FIG. 4E). Our designs mirror the behavior of naturally occurring membrane fusion proteins, such as influenza HA, in undergoing conformational rearrangements that expose the hydrophobic faces of amphipathic α-helices, allowing them to interact with membranes(4-6).

To further increase the pH of disassembly without altering the putative membrane interacting residues, we tuned the pH-sensitivity by increasing or decreasing the overall interface affinity through mutations in the hydrophobic layers (tuning ΔG_{hydrophobic}) of design pRO-2. Consistent with Eq. 1, increasing ΔG_{hydrophobic} through the A54M substitution decreases the transition pH, whereas weakening ΔG_{hydrophobic} with the I56V substitution increases the transition pH to approximately 5.8 (FIG. 5A). Neither of the mutations substantially affect the cooperativity of the transition (FIG. 5B). CD monitored denaturation experiments showed that A54M increases stability and I56V decreases stability, as expected (FIG. 15). Similar tuning of the heterodimer design pRO-4 with the destabilizing mutations L23A/V130A increased the pH transition point of disassembly from pH ˜4 to pH ˜4.6 (FIG. 10).

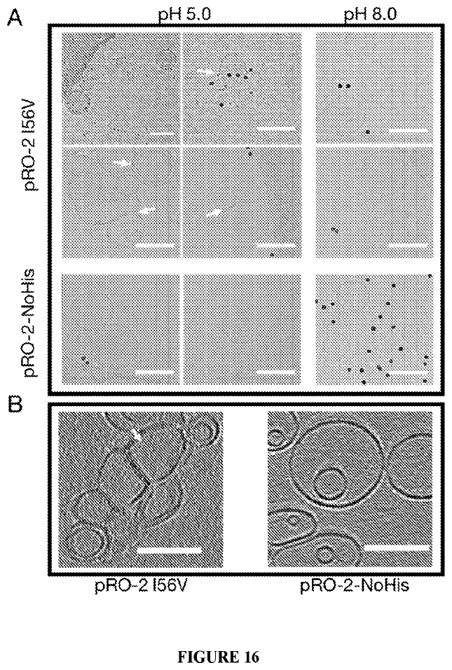

To characterize the physical interactions between protein and membranes, and the mechanism of membrane disruption, purified proteins were chemically conjugated to gold nanoparticles and visualized by cryo-electron microscopy and tomography. Design pRO-2 I56V, which has a higher transition pH (FIG. 5A), also has increased liposome permeabilization activity (Figure SB); it directly interacts with liposomes at pH 5 but not at pH 8, while the non-pH-responsive design pRO-2-noHis shows no interactions with liposomes at either pH (FIG. 5C and FIG. 16). We observed widespread membrane deformation and disruption of the lipid bilayer with design pRO-2 I56V and pRO-2 at pH 5 along with association of protein conjugated gold nanoparticles to liposomes (FIG. 5C and FIG. 16). At either pH, pRO-2-noHis and pRO-2 I56V at pH 8, there were no signs of membrane deformation or disruption and protein conjugated gold nanoparticles wee well dispersed and did not associate to the membrane (FIG. 5C and FIG. 16). At pH 5, design pRO-2 I56V causes significant deformation of the liposomal membrane and induces formation of tight extended interfaces between liposomes, we observed density at these interfaces that likely corresponds to pRO-2 I56V (FIG. 5C and FIG. 16).

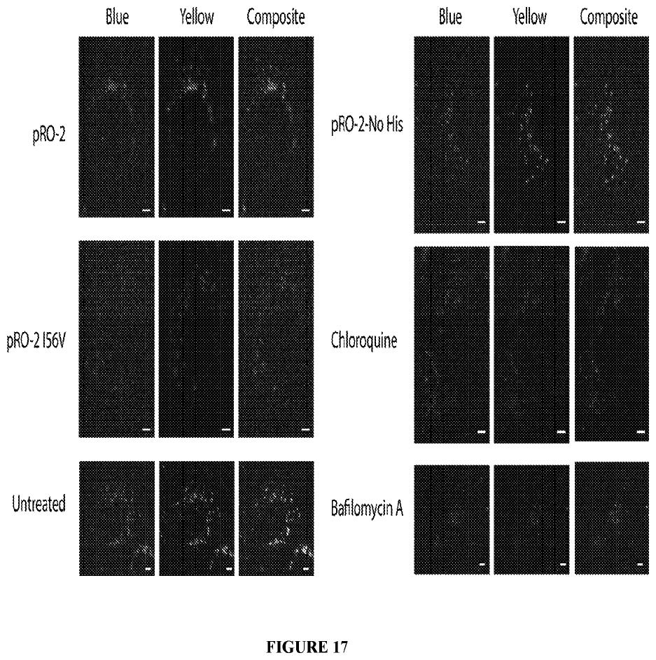

We next investigated the behavior of the designed proteins in the low pH environment of the mammalian cell endocytic pathway. Internalized proteins are either recycled back or destined for degradation through fusing with lysosomes that contain hydrolytic enzymes that are activated at round pH 5(33). To test their behavior in the endocytic pathway, we expressed the pRO-2 trimers as fissions to +36GFP(34, 35) to facilitate both fluorescent imaging and endocytosis; these fusions also showed signs of pH-induced liposome disruption by cryo-electron microscopy and tomography (FIG. 5D). Following addition to U2-OS cells, +36GFP fusions of pRO-2 and I56V colocalize with lysosomal membranes and are not degraded, whereas pRO-2-noHis is not observed in lysosomes (FIG. 5E-F). I56V, which is the most pH-sensitive and membrane active design in this study (FIG. 5A-C), is the most strongly colocalized with the lysosomal membrane (FIG. 5F). We hypothesize that pRO-2 and I56V disassemble in the lower pH environment of the lysosome and endosome, and interact with membranes to cause proton leakage and neutralization, preventing degradation; pRO-2-noHis is not pH-responsive nor membrane active and is presumably degraded by the lysosomes. To test this hypothesis, U2-OS loaded with dye to track pH (LysoSensor Yellow/Blue DND-160) were incubated for one hour with pRO-2 (5 μM), pRO-2 I56V (5 μM), or pRO-2-noHis (5 μM); design pRO-256V raises the lysosomal pH compared to pRO-2-noHis and normal cell controls (FIG. 5G and FIG. 17). Design pRO-2 I56V produces larger changes in lysosomal pH than two drugs. Bafilomycin A and Chloroquine, known to neutralize lysosomal pH (FIG. 5G).

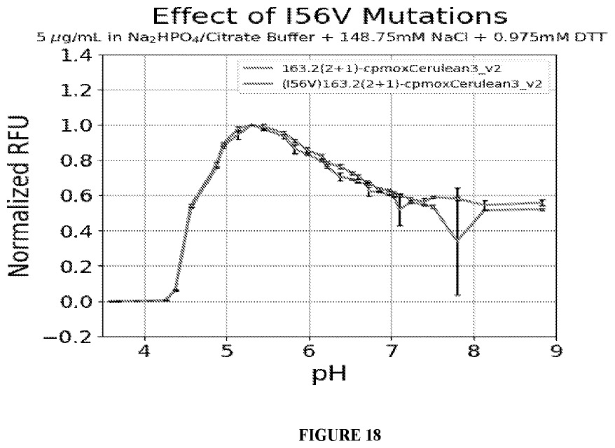

As shown in FIG. 18, the increase in fluorescence between pH 8.0 and 5.3 is shifted towards lower pH for the 163.2(2+1)-cpmoxCerulean3_v2 construct (cyan) compared with the (I56V)163.2(2+1)-cpmoxCerulean3_v2 construct (blue), which supports the theoretical model that reduced interface energy of hydrophobic layers (ΔG_{hydrophobic}) in the helical bundle due to the isoleucine-to-valine mutations increases the pH at which the helical bundle unfolding transition occurs.

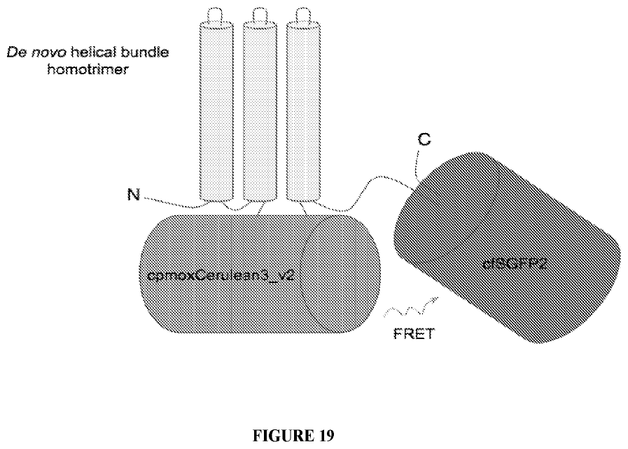

As shown in FIG. 19, at high pH, the helical bundle trimer (grey) is associated, and the cpmoxCerulean3_v2 (cyan) acts as a FRET donor to the C-terminal cfSGFP2 (green), which acts as a FRET acceptor, producing a quantifiable FRET signal. At low pH, the helical bundle timer dissociates due to histidine residues at the trimer interface becoming protonated, the conformational change of which is coupled to the cpmoxCerulean3_v2 FRET donor increasing in fluorescence brightness. The cpmoxCerulean3_v2 has a low pKₐ of unfolding, while the cfSGFP2 has a high pKₐ of unfolding, so at low pH the cpmoxCerulean3_v2 remains folded and the cfSGFP2 unfolds reducing its ability to act as a FRET acceptor. Thus, at low pH, because the FRET donor increases in fluorescence brightness while the FRET acceptor decreases in fluorescence brightness, the overall FRET signal is reduced at low pH. The described mechanism allows the designed conformational change of the helical bundle upon pH change to be coupled to measurable fluorescence readouts.

pH-dependent membrane disruption ability can be conferred to other proteins via fusion at the n-terminus of asymmetrized single-chain pH trimers. In this example, Asym206TEVAnti (magenta) was fused to a nanoparticle and is expressed and purified from E. Coli. Single-chain asymmetrized pH-responsive trimers fused to nanoparticles exhibited pH-dependent lipolysis equal to and greater than pRO2.3 (data not shown). Proteins were mixed with liposomes encapsulating self-quenching sulforhodamine B (SRB) fluorescent dye. Liposome disruption was measured by measuring fluorescence of released and dequenched of dye leaked from disrupted membranes on a spectrofluorometer.

### Conclusions

It was not previously clear how to achieve the high cooperativity that allows proteins to dramatically alter function in response to small changes in the environment. Our results now clearly answer the latter question in the affirmative—The complete loss of trimer pRO-2 over a very narrow pH range in the present disclosure demonstrates that such high cooperativity has been achieved. Furthermore, the disclosure further demonstrates the ability to systematically tune the set point and cooperativity of the conformational change.

The modular and tunable pH set point and cooperativity of our designed homo-oligomers, together with their liposome permeabilizing activity, makes them attractive for delivery of biologics into the cytoplasm through endosomal escape. Delivery methods relying on cell penetrating peptides, supercharged proteins, and lipid-fusing chemical reagents can be toxic because of nonspecific interactions with many types of membranes in a pH-independent manner.

### REFERENCES

1. C. B. Anfinsen, Principles that govern the folding of protein chains. Science. 181, 223-230(1973).
2. P.-S. Huang, S. E. Boyken, D. Baker, The coming of age of de novo protein design. Nature. 537, 320-327 (2016).
3. G. J. Rocklin et al., Global analysis of protein folding using massively parallel design, synthesis, and testing. Science. 357, 168-175 (2017).
4. C. M. Ca, P. S. Kim, A spring-leaded mechanism for the conformational change of influenza hemagglutinin. Cell. 73, 823-832 (1993).
5. J. M. White. S. E. Delos, M. Brecher, K. Schornberg, Structures and mechanisms of viral membrane fusion proteins: multiple variations on a common theme. Crit. Rev. Biochem. Mol. Biol. 43, 189-219(2008).
6. C. M. Mair, K. Ludwig, A. Herrmann, C. Sieben, Receptor binding and pH stability—how influenza A virus hemagglutinin affects host-specific virus infection. Biochimica et Biophysica Acta (BBA)—Biomembranes. 138, 1153-1168 (2014).
7. M. L. Murtaugh, S. W. Fanning, T. M. Sharma, A. M. Terry, J. R. Horn, A combinatorial histidine scanning library approach to engineer highly pH-dependent protein switches. Protein Sci. 20, 1619-1631 (2011).
8. E.-M. Strauch. S. J. Fleishman, D. Baker, Computational design of a pH-sensitive IgG binding protein. Proc. Nat. Acad. Set. U.S.A. 111, 675-680 (2014).
9. N. Gera, A. B. Hill, D. P. White, R. G. Carbonell, B. M. Rao, Design of pH sensitive binding proteins from the hyperthermophilic Sso7d scaffold. PLoS One. 7, e48928 (2012).
10. M. Dalmau, S. Lim, S.-W. Wang, pH-triggered disassembly in a caged protein complex. Biomacromolecules. 10, 3199-3206 (2009).
11. T. Igawa et al., Antibody recycling by engineered pH-dependent antigen binding improves the duration of antigen neutralization. Nat. Biotechnol. 28, 1203-1207 (2010).
12. K. Wads, T. Mizuno, J.-I. Oku, T. Tanaka. pH-induced conformational change in an alpha-helical coiled-coil is controlled by His residues in the hydrophobic core. Protein Pept. Lett. 10, 27-33 (2003).
13. R. Lizatović et al., A De Novo Designed Coiled-Coil Peptide with a Reversible pH-Induced Oligomerization Switch. Structure. 24, 946-935 (2016).
14. K. Pagel et al., Random Coils, β-Sheet Ribbons, and α-Helical Fibers: One Peptide Adopting Three Different Secondary Structures at Will. J. Am. Chem. Soc. 128, 2196-2197 (2006).
15. C. Minelli, J. X. Liew, M. Muthu, H. Andresen, Coiled coil peptide-functionalized surfaces for reversible molecular binding. Soft Matter. 9, 5119-5124 (2013).
16. J. Aupič, F. Lapenta, R. Jerala, SwitCCh: Metal-site design for controlling the assembly of a coiled-coil homodimer. Chembiochem (2018), doi:10.1002/cbic.201800578.
17. Y. Zhang et al., Computational design and experimental characterization of peptides intended for pH-dependent membrane insertion and pore formation. ACS Chem. Biol. 10, 1082-1093 (2015).
18. S. E. Boyken et al., De novo design of protein homo-oligomers with modular hydrogen-bond network-mediated specificity. Science. 352, 680-687 (2016).
19. F. H. C. Crick. The Fourier transform of a coiled-coil. Acta Crystallogr. 6, 685-689 (1953).
20. G. Grigoryan, W. F. Degrado, Probing designability via a generalized model of helical bundle geometry. J. Mol. Biol. 405, 1079-110 (2011).
21. A. Leaver-Fay et al., in Method in Enzymology, M. L. Johnson, L. Brand, Eds. (Academic Press, 2011), vol. 487, pp. 545-574.
22. B. Kuhiman, D. Baker, Native protein sequences are close to optimal for their structures. Proc. Natl. Acad. Sci. U.S.A. 97, 10383-10388 (2000).
23. P.-S. Huang et al., High thermodynamic stability of parametrically designed helical bundles. Science. 346, 481-485 (2014).
24. S. Mehmood, T. M. Allison, C. V. Robinson, Mass spectrometry of protein complexes: from origins to applications. Annu. Rev. Phys. Chem. 66, 453-474 (2015).
25. E. Boer Erba, K. Barylyuk, Y. Yang. R. Zenobi, Quantifying protein-protein interactions within noncovalent complexes using electrospray ionization mass spectrometry. Anal. Chem. 83, 9251-9259 (2011).
26. N. Leloup et al., Low pH-induced conformational change and dimerization of sortilin triggers endocytosed ligand release. Nat. Commun. 8, 1708 (2017).
27. Y.-B. Hu, E. B. Dammer, R.-J. Ren, G. Wang, The endosomal-lysosomal system: from acidification and cargo sorting to neurodegeneration. Transl. Neurodegener. 4, 18 (2015).
28. M. Grabe, G. Oster, Regulation of organelle acidity. J. Gen. Physiol. 117, 329-344 (2001).
29. K. N. Dyer et al., High-throughput SAXS for the characterization of biomolecules in solution: a practical approach. Methods Mol. Biol. 1491, 245-258 (2014).
30. S. Classen et al., Implementation and performance of SIBYLS: a dual endstation small-angle X-ray scattering and macromolecular crystallography beamline at the Advanced Light Source. J. Appl. Crystallogr. 46, 1-13(2013).
31. E. Eiriksdóttir, K. Konate, U. Langel, G. Divita, S. Deshayes, Secondary structure of cell-penetrating peptides controls membrane interaction and insertion. Biochim. Biophys. Acta. 1798, 1119-1128 (2010).
32. L. Gui, K. K. Lee, in Influenza Virus: Methods and Protocols, Y. Yamauchi, Ed. (Springer New York, N.Y., N.Y., 2918), pp. 261-279,
33. C. S. Pillay, E. Elliott, C. Dennison, Endolysosomal proteolysis and its regulation. Biochem. J. 363, 417-429 (2002).
34. M. Li et al., Discovery and characterization of a peptide that enhances endosomal escape of delivered proteins in vitro and in vivo, J. Am. Chem. Soc. 137, 14084-14093 (2015).
35. M. S. Lawrence, K. J. Phillips, D. R. Liu, Supercharging proteins can impart unusual resilience. J. Am. Chem. Soc. 129, 10110-10112 (2007).
36. J. Monod, J. Wyman, J.-P. Changeux, in Selected Papers In Molecular Biology by Jacques Monod, A. Lwoff, A. Ullmann, Eds. (Academic Press, 1978), pp. 593-423.
37. E. L. Snyder, S. F. Dowdy, Cell penetrating peptides in drug delivery. Pharm. Res. 21, 389-393 (2004).
38. Y. S. Choi, M. Y. Lee, A. E. David, Y. S. Park, Nanoparticles for gene delivery: therapeutic and toxic effects. Molecular & Cellular Toxicology. 10, 1-8 (2014).

### Materials and Methods

### Computational Design Methods

Backbone Sampling:

Oligomeric protein backbones with an inner and outer ring of α-helices were produced by systematically varying helical parameters using the Crick generating equations (19, 20). Ideal values were used for the supercoil twist (ω₀) and helical twist (ω₁)(19, 20). Starting points for the superhelical radii were chosen based on successful previous designs (18) and the helical phase (Δϕ₁) was sampled from 0° to 90° with a step size of 10°. The offset along the z-axis (Z-offset) for the first helix was fixed to 0 as a reference point, with the rest of the helices independently sampled from −1.51 Å to 1.51 Å, with a step size of 1.51 Å. For heterodimer designs, supercoil phases (Δϕ₀) were fixed at 0° 90°, 180° and 270°, respectively, for the four helices. The inner and outer helices were connected by short, structured loops as described previously (18). To find backbones that could accommodate more than two histidine networks, a second round of parametric design was performed with finer sampling around the helical parameters of the initial designs. (Note: because the inner and outer helices have different superhelical radii, the repeating geometric cross sections of the helical bundle are not always perfect geometric repeats along the z-axis; hence, because of the geometric sensitivity of hydrogen bonding, finer sampling was required to find backbones that could accommodate the same histidine hydrogen bond networks at multiple layers/cross sections).

Design of Histidine Networks:

the HBNet™ (18) method in Rosetta™ (21) was extended to include program code that allowed for the selection of hydrogen bond networks that contain at least one histidine at oligomeric interfaces, and also the option to select for cases where the histidine residue accepts a hydrogen bond across the oligomeric interface. HBNet™ was used to select backbones that could accommodate 1-4 such networks in the homotrimeric and heterodimeric backbones.

Rosetta™ Design Calculations:

To design the sequence and sidechain rotamer conformations for the rest of the protein surrounding the hydrogen bond networks, the network residues were constrained using AtomPair™ constraints on the donors and acceptors of the hydrogen bonds and RosettaDesign™ calculations carried out, and best designs selected.

Design Strategy to Tune pH Set Point and Cooperativity Via Modular Placement of the Histine Network:

Once successful designs were identified, HBNet™ was used to generate all possible combinations of hydrogen bond network placement for the existing networks within the backbone of that design; for each, the amino acid sequence and side chain rotamer conformations were optimized around those placed networks as described above. From these combinations for pRO-2, designs pRO-2.1-2.5 (FIG. 3) were selected based on placement of networks m and l relative to the hydrophobic layers, n, to test our tuning strategy. Design pRO-2mutants I56V and A54M were designed rationally without any computational design.

### Protein Expression and Purification

Plasmids containing synthetic genes that encode the designed proteins were ordered through Genscript, Inc. (Piscataway, N.J., USA), cloned into the NdeI and XhoI sites of either pET2I-NESG or pET-28b vectors (see table 3). Plasmids were transformed into chemically competent E. coli expression strains BL21(DE3) Star (Invitrogen) or Lemo™21(DE3) (New England Biolabs). Following transformation, single colonies were picked from agar plates and grown overnight in 5 m starter cultures of Luria-Bertani (LB) medium containing 50 μg/mL carbenicillin (for pET21-NESG vectors) or kanamycin (for pET-28b vectors) with shaking at 225 rpm for 12-18 hours at 37° C. 5 ml starter cultures were added to 500 ml TBM-3052 with antibiotic for expression by autoinduction; cells were grown at 37° C. for 4-7 hours and temperature was dropped to 18° C. overnight. After 18-24 hours, cells were harvested by centrifugation for 15 minutes at 5000 ref at 4° C. and resuspended in 20 ml lysis buffer (25 mM Tris pH 8.0 at room temperature, 300 mM NaCl, 20 mM Imidazole).

Cells were lysed by microfluidization in the presence of 1 mM PMSF. Lysates were clarified by centrifugation at 24,000 ref at 4° C. for at least 30 minutes. Proteins were purified by Immobilized metal affinity chromatography (IMAC): supernatant was applied to Ni-NTA (Qiagen) columns pre-equilibrated in lysis buffer. The column was washed twice with 15 column volumes (CV) of wash buffer (25 mM Tris pH 8.0 at room temperature, 300 mM NaCl, 40 mM Imidazole), followed by 3-5 CV of high-salt wash buffer (25 mM Tris pH 8.0 at room temperature, 1 M NaCl, 40 mM Imidazole) then an additional 15 CV of wash buffer. Protein was eluted with 250 mM Imidazole, and buffer-exchanged into 25 mM Tris pH 8.0 and 150 mM NaCl without imidazole for cleavage of the N-terminal hexahistidine tag by purified hexahistidine-tagged TEV protease (with the exception of design pRO-1, which was cleaved using restriction grade thrombin (EMD Millipore 69671-3) at room temperature for 4 hours or overnight, using a 1:3000 dilution of enzyme into sample solution). A second Ni-NTA step was used to remove hexahistidine tag, uncleaved sample and the hexahistidine-tagged TEV protease, and the cleaved proteins were then concentrated and further purified by gel filtration using FPLC and a Superdex™ 75 Increase 10/300 GL (GE) size exclusion column in 25 mM Tris pH 8.0 at room temperature, 150 mM NaCl, and 2% glycerol.

### Buffets for Varying pH

For low-pH experiments involving circular dichroism (CD), small-angle X-ray scattering (SAXS), and size exclusion chromatography (SEC), Na₂PO₄-Citrate buffer was used to ensure that a single buffer system could be used that was stable over the entire pH range to be tested. Buffers were made using established ratios of stock solutions of 0.2 M Na₂PO₄ and 0.1 M Citrate; final pH was adjusted using hydrochloric acid (HCl) or sodium hydroxide (NaOH) if needed. For SAXS and SEC, 150 mM NaCl and 2% glycerol were added. Native mass spectrometry experiments required the use of ammonium acetate buffer, and pH was adjusted using acetic acid, with the final pH value measured (see Native Mass Spectrometry section below). For liposome disruption assays, 10 mM Tris, 150 mM NaCl, 0.02% NaN₃, pH 8.0 was used and pH was changed by rapid acidification using 10 mM HEPES, 150 mM NaCl, 50 mM Citrate and 0.02% NaN₃ buffer at pH 3.0 as described previously (32), and final pH values were measured (see Fluorescence Dequenching Liposome Leakage Assay section below).

Hexahistidine tag was removed for all experiments that tested the effect of pH.

### Circular Dichroism (CD)

CD wavelength scans (260 to 195 nm) and temperature melts (25 to 95° C.) were measured using a JASCO™ J-1500 or an AVIV™ model 420 CD spectrometer. Temperature melts monitored absorption signal at 222 nm and were carried out at a heating rate of 4° C./min; protein samples were at 0.25 mg/mL in either phosphate buffered saline (PBS) pH 7.4 or Na₂PO₄-Citrate at indicated pH values (see Buffers systems for varying pH). Guanidinium chloride (GdmCl) titrations were all performed on an AVIV 420 spectrometer with an automated titration apparatus using either PBS pH 7.4 or Na₂PO₄-Citrate buffers at indicated pH at room temperature, monitoring helical signal at 222 nm, using a protein concentration of 0.025 mg/mL in a 1 cm cuvette with stir bar. Each titration consisted of at least 30 evenly distributed concentration points with one minute mixing time for each step. Titrant solution consisted of the same concentration of protein in the same buffer system plus GdmCl; GdmCl concentration of starting solutions was determined by reactive index.

### Native Mass Spectrometry

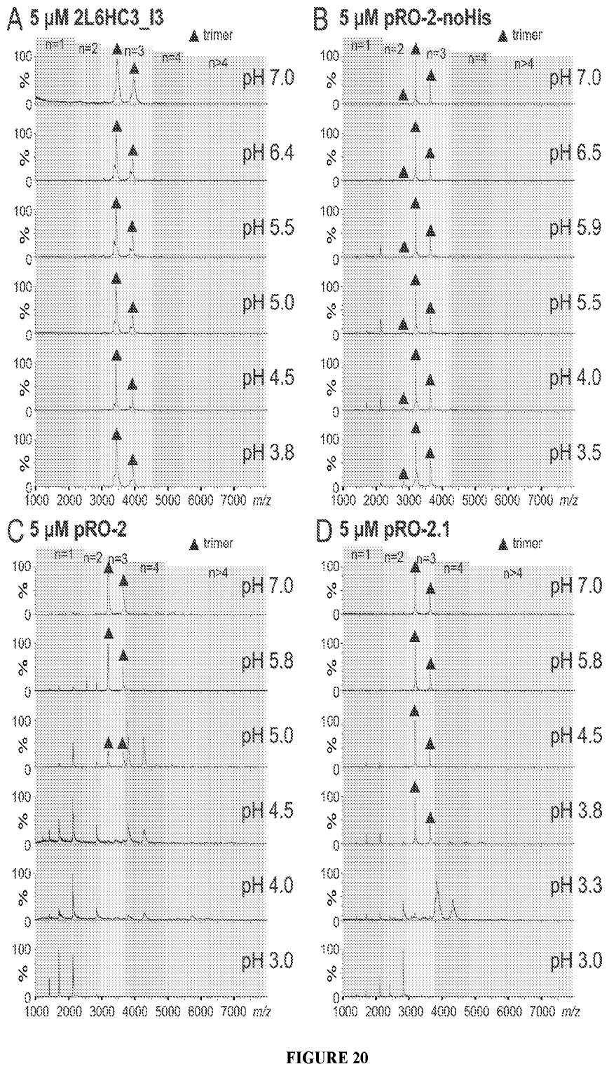

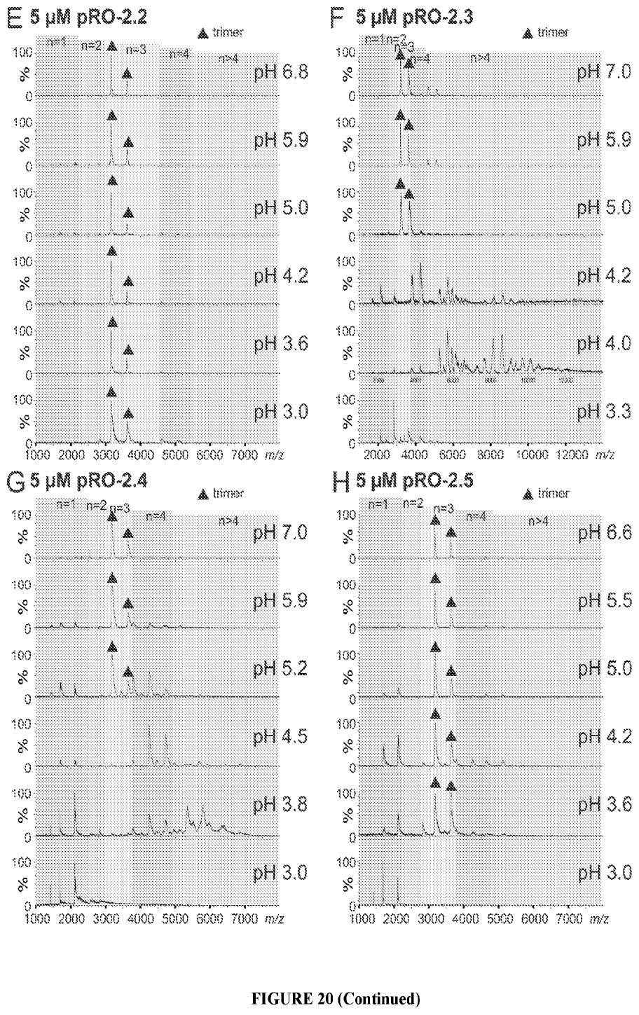

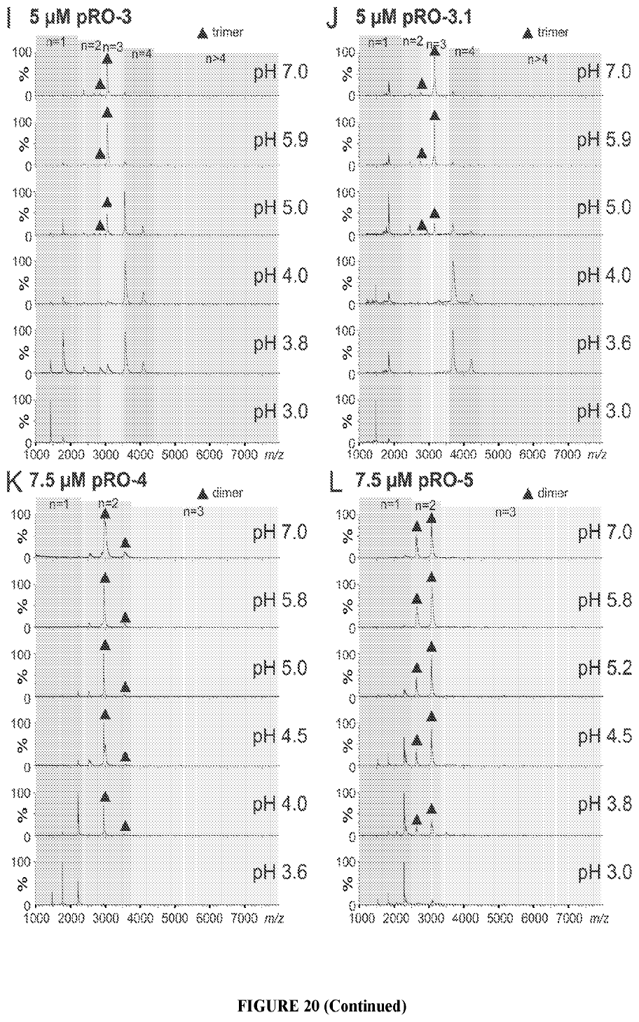

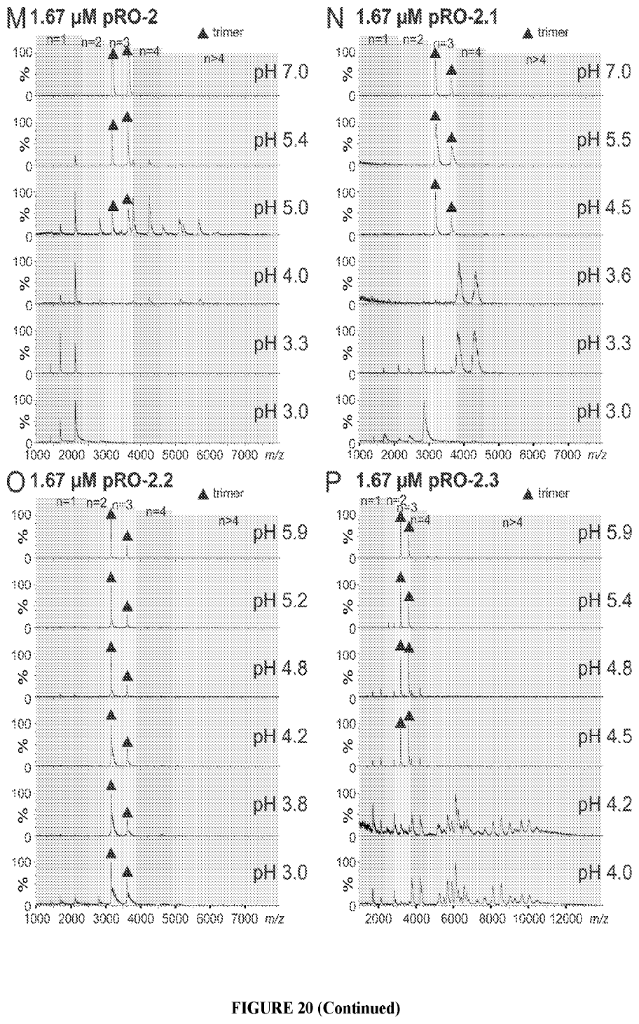

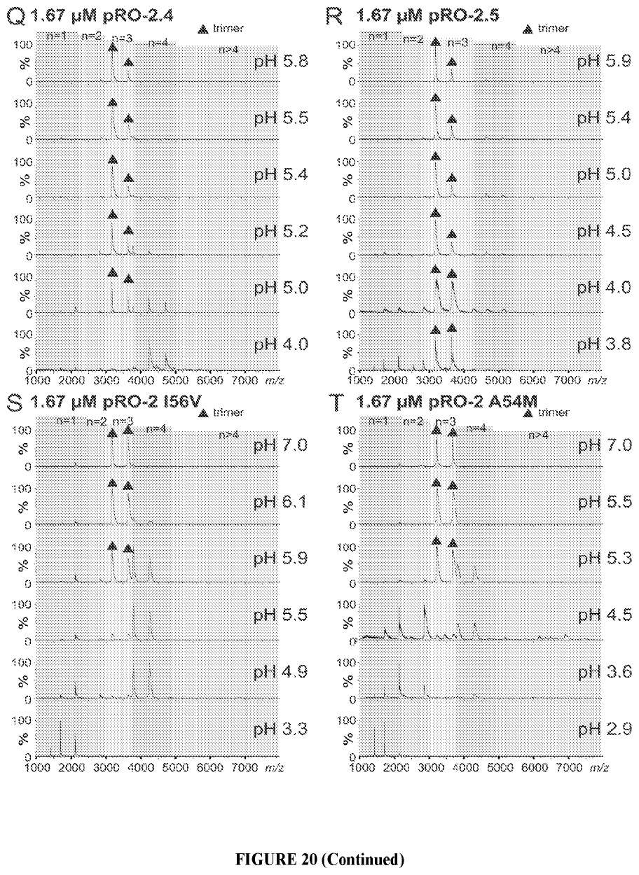

Samples were buffer exchanged twice into 200 mM ammonium acetate (NH₄Ac; MilliporeSigma) using Micro Bio-Spin P-6 columns (Bio-Rad). Protein concentrations were determined by UV absorbance using a Nanodrop 2000c spectrophotometer (Thermo Fisher Scientific) and diluted to make up a 10-fold stock solution (50 μM and 16.7 μM monomer and trimer concentration, respectively). 1 μL of this solution was mixed with 9 μL 200 mM NH₄Ac/50 mM triethylammonium acetate (TEAA; MilliporeSigma), adjusted with acetic acid (Fisher Scientific) to obtain the desired final pH and incubated on ice for 30 min. For experiments to test for the reversibility of disassembly, the pH was subsequently increased either by addition of ammonia or by buffer-exchange to 200 mM NH₄Ac/50 mM TEAA (pH 7.0) via ultrafiltration (Amicon Ultra, MWCO 3 kDa). 5 μL samples were filled into an in-house pulled glass capillary and ionized by nESI at a monomer or a trimer concentration of 5 μM or 1.67 μM respectively. All pH titration data were acquired on an in-house modified SYNAPT® G2 HDMS (Waters Corporation) with a surface-induced dissociation (SID) device incorporated between a truncated trap traveling wave ion guide and the ion mobility cell (39). The following instrument parameters were used spray voltage 0.9-1.3 kV; sampling cone, 20 V; extraction cone, 2 V; source temperature, room temperature; trap gas flow, 4 mL/min; trap bins, 45V. The data were processed with MassLynx™ v4.1 and DriftScope™ v2.1. Smoothed mass spectra (mean; window 20; number of smooths 20) are shown in FIGS. 9 and 20. For relative quantification, charge state series were extracted from DriftScope™, and smoothed spectra (mean; window 20; number of smooths 20) were integrated.

### Small-Angle X-Ray Scattering (SAXS)

Samples were purified by gel filtration in either 25 mM Tris pH 8.0 at room temperature, 150 mM NaCl, and 2% glycerol, or Na₂PO₄-Citrate buffer at indicated pH with 150 mM NaCl and 2% glycerol. For each sample, data was collected for at least two different concentrations to test for concentration-dependent effects; “high” concentration samples ranged from 4-10 mg/ml and “low” concentration samples ranged from 1-5 mg/ml (table 5). Fractions preceding the void volume of the column, or from the flow-through during concentration using spin concentrators (Millipore), were used as blanks for buffer subtraction. SAXS measurements were made at the SiBYLS™ 12.3.1 beamline at the Advanced Light Source. The X-ray wavelength (λ) was 1.27 Å and the sample-to-detector distance of the Mar165 detector was 1.5 m, corresponding to a scattering vector q (q=4π*sin(θ/λ) where 2θ is the scattering angle) range of 0.01 to 0.59 Å⁻¹. Data sets were collected using 34 0.2 second exposures over a period of 7 seconds at 11 keV with protein at a concentration of 6 mg/mL. The light path is generated by a super-bend magnet to provide a 1012 photons/sec flux (1 Å wavelength) and detected on a Pilatus3 2M pixel array detector. Each sample is collected multiple times with the same exposure length, generally every 0.3 seconds for a total of 10 seconds resulting in 30-34 fames per sample. These individual spectra were averaged together over each of the Gunier, Parod, and Wide-q regions depending on signal quality over each region and frame using the FrameSlice™ web server. The averaged spectra for each sample were analyzed using the ScÅtter™ software package as previously described (29, 40). FoXS™ (41,42) was used to compare design models to experimental scattering profiles and calculate quality of fit (X) values.

### X-Ray Crystallography

Purified protein samples were concentrated to 13 ng/ml for pRO-2.3 and 17 mg/ml for pRO-2 Sin 20 mM Tris pH 8.0 at room temperature with 100 mM NaCl. Samples were screened with a 5-position deck Mosquito crystallization robot (ttplabtech) with an active humidity chamber, utilizing JCSG Core™ I-IV screens (Qiagen). Crystals were obtained after 2 to 14 days by the sitting drop vapor diffusion method with the drops consisting of a 1:1, 2:1 and 1:2 mixture of protein solution and reservoir solution. The conditions that resulted in the crystals used for structure determination are as follows: pRO-2.3 crystallized in JCSG-I B7, which consists of 0.2M di-sodium tartrate and 20% w/v PEG 3350; pRO-2.5 crystalized in JCSG-I A9, which consists of 0.2 M Potassium acetate and 20% w/v PEG 3350.

### X-Ray Data Collection and Structure Determination

Protein crystals were looped and placed in reservoir solution containing 20% (v/v) glycerol as a cryoprotectant, and flash-frozen in liquid nitrogen. Datasets were collected at the Advanced Light Source at Lawrence Berkeley National Laboratory with beamlines 8.2.1 and 8.2.2. Data sets were indexed and scaled using XDS (43). Phase information was obtained by molecular replacement using the program PHASER™ (44) from the Phenix software suite (45); computational design models were used for the initial search. Following molecular replacement, the models were improved using Phenix™ autobuild (46); efforts were made to reduce model bias by setting rebuild-in-place to false, and using simulated annealing and prime-and-switch phasing. Iterative rounds of manual building in COOT™ (47) and refinement in Phenix™ were used to produce the final models. Due to the high degree of self-similarity inherit in coiled-coil-like proteins, datasets for the reported structures suffered from a high degree of pseudo translational non-crystallographic symmetry, as report by Phenix™.Xtriage, which complicated structure refinement and may explain the higher than expected R-values reported. RMSDs of bond lengths, angles and dihedrals from ideal geometries were calculated using Phenix™ (45). The overall quality of the final models was assessed using MOLPROBITY (48). Table 4 summarizes diffraction data and refinement statistics.

### Liposomes Preparation and Characterization

Liposomes composed of DOPC (1,2-dioleoyl-sn-glycero-3-phosphocholine), DOPC with 25% cholesterol (molar ratio to DOPC), 3:1 DOPC:POPS (1-palmitoyl-2-oleoyl-sn-glycero-3-phospho-L-serine), and 3:1 DOPC:POPS with 25% cholesterol were prepared identically to a final concentration of 5 mM total lipid as previously described (32); lipids from Avanti Polar Lipids. Lipids solubilized in chloroform were dried under nitrogen gas and stored under vacuum for a minimum of 2 hours to remove residual solvent. The dried lipid film was the resuspended in Tris buffer (10 mM Tris, 150 mM NaCl, and 0.02% NaN₃ pH 8.0) containing 25 mM Sulforhodamine B (SRB) fluorophore (Sigma) and subjected to 10 sequential freeze thaw cycles in liquid nitrogen. Liposomes were extruded 29 times through 100 nm pore size polycarbonate filters (Avanti Polar Lipids) and purified from free fluorophore using a PD-10 gel filtration column (GE Healthcare) into storage buffer (10 mM Tris, 150 mM NaCl, and 0.02% NaN₃ pH 8.0). Liposome size and homogeneity was analyzed by dynamic light scattering (DLS) using a Dynapro Nanostar™ DLS (Wyatt Technologies). On average liposome diameter ranged from 120-130 nm with low polydispersity. Liposomes were stored at 4° C. and used within 5 days of preparation.

### Fluorescence Dequenching Liposome Leakage Assay

Liposome disruption and content leakage was analyzed by fluorescence spectroscopy as previously described (32). Liposomes containing SRB fluorophore at self-quenching concentrations were incubated with 2.5 μM peptide, with respect to monomer, at 24° C. and pH 8.0 in Tris buffer (10 mM Tris, 150 mM NaCl, 0.02% NaN₃, pH 8.0) for 10 minutes. The solution was rapidly acidified to the target pH by addition of a fixed volume of acidification buffer and incubated for 20 minutes. Acidification buffers are mixtures of the Tris pH 8.0 buffer and citrate buffer pH 3.0 (10 mM HEPES, 150 mM NaCl, 50 mM Citrate and 0.02% NaN₃ pH 3.0) in empirically determined ratios to achieve the target pH. SRB fluorescence is independent of pH within the ranges used here. Finally. Triton X-100 (Sigma) was added to a final concentration of 1% to fully disrupt liposomes. Liposome disruption as indicated by content leakage and SRB dequenching was normalized using the formula [F_{ω}−F₍₀₎]/[F_{(Max)}−F₍₀₎] where F₍₀₎ is the average fluorescence intensity before acidification and F_{(Max)} is the average fluorescence intensity ater addition of Triton X-100. All measurements were collected on a Varian Cary Eclipse spectrophotometer using an excitation/emission pairing of 1 565/586 and 2.5 nm slit widths at 24° C. Any data plotted together was collected using the sum batch of liposomes.

### Cryo-EM Specimen Preparation and Imaging

Designs pRO-2, pRO-2 I56V, and pRO-2-noHis were chemically conjugated to 10 nm Gold nanoparticles according to manufacturer's instructions, ensuring all gold nanoparticles were conjugated to protein. The conjugation reactions were performed immediately prior to use for electron microscopy imaging. For each design pRO-2, pRO-2 I56V, and pRO-2-noHis a solution of 2.5 μM purified protein, 0.125 μM gold-conjugated protein, and 1 mM DOPC liposomes was applied to glow-discharged C-Flat 2/2-2C-T holey carbon grids (Protochips, Inc.) and acidified on the grid by addition of HEPES-citrate buffer. The grids were prepared using a Vitrobot Mark IV (FEI) at 4 C and 100% humidity before being plunge-frozen in ethane cooled with liquid nitrogen.

Electron micrographs were collected using a Tecnai G2 Spirit™ Transmission Electron Microscope (FEI) operated at 120 kV and equipped with a 4k×4k Gatan Ultrascan CCD camera at a nominal magnification of 26,000× or a Tecnai TF-20 Transmission Electron Microscope (FEI) operated at 200 kV equipped with a K2 Summit Direct Electron Detector (Gatan).

Projection micrographs collected on the TF-20 were captured with the detector operating in counting mode. Specimens were imaged at 14,500 magnification, giving a pixel size of 0.254 nm, with a dose of ˜18e−/Å² across 75 200 ms movie frames. Data were collected in a semi-automated fashion using Leginon™ (49) and micrograph movie frames were aligned using MotionCor2™ (50). Leginon™ was used to collect tomography tilt series from −48 to +48 degrees bidirectionally in 3 degree increments with a total accumulated dose of ˜100 e−/Å². Reconstructions were processed using etomo in the IMOD™ software suite (31) with CTF parameters estimated from CTFFIND4™ (52). Reconstructed tomograms were visualized and measurements were made using ImageJ™ (53).

### Cell Culture, Plating, and Transfection

U-2 OS (ATCC) cells were cultured in DMEM supplemented with 10% (v/v) inactivated FBS (Corning), 2 mM glutamine, penicillin (100 IU/mL), and streptomycin (100 μg/mL) at 37° C. and 5% CO2. The glass-bottom coverslip chambers were pre-coated with 500 μg/mL of Matrigel (Corning). Transfection of LAMP1-HaloTag™ was performed using Lonza Nucleofector system according to the manufacturer's specifications. After overnight of recovery and expression, the cells expressing LAMP1-HaloTag™ were labeled with 100 nM JF646-HTL for 30 minutes and washed three times with pre-warmed DMEM medium.

### Live Cell Experiments

The final concentration of 5 μM+36GFP fusion proteins was incubated with the LAMP1-HaloTag™ expressing U-2 OS cells on a pre-coated coverslip for 1 hr. Cells were fixed with 4% paraformaldehyde for 20 min at room temperature (RT) and quenched/rinsed with PBS supplemented with 30 mM glycine. Then, the coverslips were mounted on FluoroSave™ (Millipore). For pH measurement of the lysosome, LysoSensor™ Yellow/Blue DND-160 was incubated at 1 mg/mL overnight and washed twice prior to imaging (54). The final concentration of 5 μM protein was incubated with the LAMP1-HaloTag expressing U2-OS cells that were loaded with 1 mg/mL LysoSensor™ Yellow/Blue DND-160 for 1 hr. In separate chambers, LysoSensor™ Yellow/Blue DND-160 loaded cells were incubated with bafilomycin A1 (1 μM) and chloroquine (50 μM) for 1 hr as a control.

### Confocal Microscopy

For fixed cell confocal microscopy, a customized Nikon TiE inverted scope outfitted with a Yokogawa spinning-disk scan head (#CSU-X1) along with an Andor iXon™ EM-CCD camera (DU-897) with 100-ns exposure time was used to collect 3D images using an SR Apo TIRF 100×1.49 oil-immersion objective. Mender's coefficients were calculated in 3D with JF646 signal (LAMP-HaloTag) and +36GFP signal (corresponding proteins) using Imaris software with thresholding. Zeiss 880 equipped with AiryScan™ was also used to obtain high resolution images using a Plan-Apochromatic 63×/1.4 oil DIC objective.

For live cell confocal microscopy, Zeiss 880 was used to collect LysoSensor™ Yellow/Blue signal. LysoSensor™ Yellow/Blue was excited with a 405 nm laser, and its emission was collected into the two regions (Blue=410-499 nm Yellow=500-600 nm) using a Plan-Apochromat 63×/1.4 oil DIC objective. The ratio of the two channels was calculated using the home-built software in Matlab™.

### Visualization and Figure

All structural images for figures were generated using PyMOL™ (55).

### Theoretical Modeling and Fitting to Native Mass Spectrometry Data

Python scripts were written to generate theoretical models according Equation 1, and curve-fitting to native mass spectrometry data (FIGS. 1, 3, 5) according to Equation 2 by nonlinear least squares using curve fit from scipy.optimize. The free energy estimates for individual n, m, and l layers used in Equation 1 modeling were estimated by solving linear equations as follows: values for the free energy of folding for designs pRO-2 and variants were estimated from GdmCl denaturation experiments (FIG. 11); each of these designs have different numbers of n, m, and l layers, thus series of linear equations relating the number of each layer type to the total free energies of folding were solved to estimate dG values of the individual layers of each type. These dG estimates for the individual n, m, and l layers were then used in the theoretical modeling (Eq. 1) shown in FIG. 3C.

TABLE 4
X-ray crystallography data collection and refinement statistics.

| | pRO-2.3 (6MSQ) | pRO-2.5 (6MSR) |
|---|---|---|
| Wavelength | 0.9999 | 1 |
| Resolution range | 43.79-1.28 (1.326-1.28) | 28.7-1.55 (1.605-1.55) |
| Space group | P 63 | C 121 |
| Unit cell | 50.5663 50.5663 130.753 90 | 57.618 33.281 114.455 90 |
| | 90 120 | 99.557 90 |
| Total reflections | 429120 (15514) | 142682 (14317) |
| Unique reflections | 48463 (4882) | 31393 (3139) |
| Multiplicity | 8.8 (6.4) | 4.5 (4.6) |
| Completeness (%) | 99.8 (100.0) | 95.36 (89.40) |
| Mean I/sigma(I) | 7.83 (0.5) | 9.97 (1.49) |
| Wilson B-factor | 16.44 | 24.47 |
| R-merge | 0.117 (3.554) | 0.07484 (1.027) |
| R-meas | 0.125 (3.880) | 0.08526 (1.164) |
| R-pim | 0.042 (1.536) | 0.04017 (0.5402) |
| CC1/2 | 0.998 (0.428) | 0.995 (0.728) |
| CC* | 1 (0.701) | 0.999 (0.918) |
| Reflections used | 48462 (2888) | 31393 (2808) |
| in refinement | | |
| Reflections | 1657 (115) | 1407 (129) |
| used for R-free | | |
| R-work | 0.1726 (0.5196) | 0.2424 (0.3852) |
| R-free | 0.1944 (0.5228) | 0.2639 (0.3803) |
| CC(work) | 0.961 (0.276) | 0.954 (0.770) |
| CC(free) | 0.965 (0.253) | 0.966 (0.803) |
| Number of | 1423 | 1916 |
| non-hydrogen | | |
| atoms | | |
| macromolecules | 1172 | 1755 |
| solvent | 251 | 161 |
| Protein residues | 152 | 228 |
| RMS(bonds) | 0.007 | 0.005 |
| RMS(angles) | 0.73 | 0.83 |
| Ramachandran | 100.00 | 100.00 |
| favored (%) | | |
| Ramachandran | 0.00 | 0.00 |
| allowed (%) | | |
| Ramachandran | 0.00 | 0.00 |
| outliers (%) | | |
| Rotamer | 0.00 | 2.40 |
| outliers (%) | | |
| Clashscore | 0.84 | 3.16 |
| Average B-factor | 26.70 | 43.57 |
| macromolecules | 24.47 | 43.33 |
| solvent | 37.08 | 46.19 |
| Number or TLS | | 6 |
| groups | | |

Statistics for the highest-resolution shell are shown in parentheses.

TABLE 5
SAXS data collection and analysis.

| | | | | | | | Perod | | |
|---|---|---|---|---|---|---|---|---|---|
| | Concent | I(0) (cm⁻¹) | R_{g} (Å) | I(0) (cm⁻¹) | R_{g} (Å) | | volume | | |
| | ration | [from | [from | [from | [from | Dₘₐₓ | estimate | | |
| Design name | (mg ml⁻¹) | P(r)] | P(r)] | Guinier] | Guinier] | (Å) | (Å³) | R_{c} | Pₛ |
| pRO-2 | 5.0 | 1570 | 21.66 | 1670 | 21.97 | 72 | 50287 | 14.2 | 3.4 |
| pRO-2-noHis | 3.8 | 1070 | 21.54 | 1090 | 21.36 | 70 | 46442 | 13.7 | 3.5 |

### REFERENCES

1. L. C. B. Anfinsen, Principles that govern the folding of protein chains. Science. 191, 223-230(1973).
2. P.-S. Huang, S. E. Boyken, D. Baker, The coming of age of de novo protein design. Nature. 537, 320-327 (2016).
3. G. J. Rocklin el al., Global analysis of protein folding using massively parallel design, synthesis, and testing. Science. 357, 168-175 (2017).
4. C. M. Carr, P. S. Kim, A spring-loaded mechanism for the conformational change of influenza hemagglutinin. Cell. 73, 823-832 (1993).
5. L. M. White, S. E. Delos, M. Brecher, K. Schornberg, Structures and mechanisms of viral membrane fusion proteins: multiple variations on a common theme. Crit. Rev. Biochem. Mol. Biol. 43, 189-219 (2008).
6. C. M. Mair, K. Ludwig, A. Herrmann, C. Sieben, Receptor binding and pH stability—how influenza A virus hemagglutinin affects host-specific virus infection. Biochimica et Biophysica Acta (BBA)-Biomembranes. 1838, 1153-1168 (2014).
7. M. L. Murtaugh, S. W. Fanning, T. M. Sharma, A. M. Terry, J. R. Horn, A combinatorial histidine scanning library approach to engineer highly pH-dependent protein switches. Protein Sci. 20, 1619-1631 (2011).
8. E.-M. Strauch, S. J. Fleishman, D. Baker, Computational design of a pH-sensitive IgG binding protein. Proc. Natl. Acad. Sci. U.S.A 111, 675-680 (2014).
9. N. Gera, A. B. Hill, D. P. White, R. G. Carbonell, B. M. Rao, Design of pH sensitive binding proteins from the hyperthermophilic Sso7d scaffold. PLoS One. 7, e48928 (2012).
10. M. Dalman, S. Lim, S.-W. Wang, pH-triggered disassembly in a caged protein complex. Biomacromolecules. 10, 3199-3206 (2009).
11. T. Igawa et al., Antibody recycling by engineered pH-dependent antigen binding improves the duration of antigen neutralization. Nat. Biotechnol. 28, 1203-1207(2010).
12. K. Wada, T. Mizuno, J.-I. Oku, T. Tanaka, pH-induced conformational change in an alpha-helical coiled-coil is controlled by His residues in the hydrophobic core. Protein Pept. Lett. 10, 27-33 (2003).
13. R. Lizatović et al., A De Novo Designed Coiled-Coil Peptide with a Reversible pH-Induced Oligomerization Switch. Structure. 24, 946-955(2016).
14. K. Pagel et al., Random Coils, β-Sheet Ribbons, and α-Helical Fibers: One Peptide Adopting Three Different Secondary Structures at Will. J. Am. Chem. Soc. 128, 2196-2197 (2006).
15. C. Minelli, J. X. Liew, M. Muthu, H. Andresen. Coiled coil peptide-functionalized surfaces for reversible molecular binding. Soft Matter. 9, 5119-5124 (2013).
16. J. Aupič, F. Lapenta, R. Jerala, SwitCCh: Metal-site design for controlling the assembly of a coiled-coil homodimer. Chembiochem (2018), doi:10.1002/cbic.201800578.
17. Y. Zhang et al., Computational design and experimental characterization of peptides intended for pH-dependent membrane insertion and pore formation. ACS Chem. Biol. 10, 1082-1093 (2015).
18. S. E. Boyken el al., De novo design of protein homo-oligomers with modular hydrogen-bond network-mediated specificity. Science. 352, 680-687(2016).
19. F. H. C. Crick, The Fourier transform of a coiled-coil. Acta Crystallogr. 6, 685-689 (1953).
20. G. Grigoryan, W. F. Degrado, Probing designability via a generalized model of helical bundle geometry. J. Mol. Biol. 405, 1079-1100 (2011).
21. A. Leaver-Fay et al., in Methods in Enzymology, M. L. Johnson, L. Brand, Eds. (Academic Press, 2011), vol. 487. pp. 545-574.
22. B. Kuhlman, D. Baker, Native protein sequences are close to optimal for their structures. Proc. Natl. Acad. Sci. U.S.A. 97, 10383-10388 (2000).
23. P.-S. Huang et al., High thermodynamic stability of parametrically designed helical bundles. Science. 346, 481-485 (2014).
24. S. Mehmood, T. M. Allison, C. V. Robinson, Mass spectrometry of protein complexes: from origins to applications. Annu. Rev. Phys. Chem. 66, 453-474 (2015).
25. E. Boer Erba, K. Barylyuk, Y. Yang, R. Zenobi, Quantifying protein-protein interactions within noncovalent complexes using electrospray ionization mass spectrometry. Anal. Chem. 83, 9251-9259 (2011).
26. N. Leloup et al., Low pH-induced conformational change and dimerization of sortilin triggers endocytosed ligand release. Nat. Commun. 8, 1708 (2017).
27. Y.-B. Hu, E. B. Dammer, R.-J. Ren, G. Wang, The endosomal-lysosomal system: from acidification and cargo sorting to neurodegeneration. Transl. Neurodegener. 4, 18 (2015).
28. M. Grabe, G. Oster, Regulation of organelle acidity. J. Gen. Physiol. 117, 329-344 (2001).
29. K. N. Dyer et al., High-throughput SAXS for the characterization of biomolecules in solution: a practical approach. Methods Mol. Biol. 1491, 245-258 (2014).
30. S. Classen et al., Implementation and performance of SIBYLS: a dual endstation small-angle X-ray scattering and macromolecular crystallography beamline at the Advanced Light Source. J. Appl. Crystallogr. 46, 1-13(2013).
31. E. Eiriksdóttir, K. Konate, U. Langel, G. Divita, S. Deshayes, Secondary structure of cell-penetrating peptides controls membrane interaction and insertion. Biochim. Biophys. Acta. 1798, 1119-1128 (2010).
32. L. Gui, K. K. Lee, in Influenza Virus: Methods and Protocols, Y. Yamauchi, Ed. (Springer New York, N.Y., N.Y., 2918), pp. 261-279,
33. C. S. Pillay, E. Elliott, C. Dennison, Endolysosomal proteolysis and its regulation. Biochem. J. 363, 417-429 (2002).
34. M. Li et al., Discovery and characterization of a peptide that enhances endosomal escape of delivered proteins in vitro and in vivo, J. Am. Chem. Soc. 137, 14084-14093 (2015).
35. M. S. Lawrence, K. J. Phillips, D. R. Liu, Supercharging proteins can impart unusual resilience. J. Am. Chem. Soc. 129, 10110-10112 (2007).
36. J. Monod, J. Wyman, J.-P. Changeux, in Selected Papers In Molecular Biology by Jacques Monod, A. Lwoff, A. Ullmann, Eds. (Academic Press, 1978), pp. 593-423.
37. E. L. Snyder, S. F. Dowdy, Cell penetrating peptides in drug delivery. Pharm. Res. 21, 389-393 (2004).
38. Y. S. Choi, M. Y. Lee, A. E. David, Y. S. Park, Nanoparticles for gene delivery: therapeutic and toxic effects. Molecular & Cellular Toxicology. 10, 1-8 (2014).
39. M. Zhou, C. Huang, V. H. Wysocki, Surface-induced dissociation of ion mobility-separated noncovalent complexes in a quadrupole/time-of-flight mass spectrometer. Anal. Chem. 84, 6016-6023 (2012).
40. R. P. Rambo, J. A. Tainer, Characterizing flexible and intrinsically unstructured biological macromolecules by SAS using the Porod-Debye law. Biopolymers. 95, 559-571(2011).
41. D. Schneidman-Duhovny, M. Hammel, A. Sali, FoXS: a web server for rapid computation and fitting of SAXS proles. Nucleic Acids Res. 38, W540-4 (2010).
42. D. Schneidman-Duhovny, M. Hammel, J. A. Tainer, A. Sali, Accurate SAXS profile computation and its assessment by contrast variation experiments. Biophys. J. 105, 962-974(2013).
43. W. Kabsch, XDS. Acta Crysallogr. D Biol. Crystallogr. 66, 125-132(2010).
44. A. J. McCoy et al., Phaser crystallographic software. J. Appl. Crysallogr. 40, 658-674 (2007).
45. P. D. Adams et al., PHENIX: a comprehensive Python-based system for macromolecular structure solution. Acta Crysallogr. D Biol. Crystallogr. 66, 213-221 (2010).
46. T. C. Terwilliger et al., Iterative model building, structure refinement and density modification with the PHENIX AutoBuild wizard. Acta Crystallogr. D Biol. Crystallogr. 64, 61-69 (2008).
47. P. Emsley, K. Cowtan, Coot: model-building tools for molecular graphics. Acta Crysallogr. D Biol. Crystallogr. 60, 2126-2132 (2004).
48. I. W. Davis et al., MolProbity: all-atom contacts and structure validation for proteins and nucleic acids. Nucleic Acids Res. 35, W375-83 (2007).
49. C. Suloway et al., Automated molecular microscopy: the new Leginon system. J. Struct. Biol. 151, 41-40 (2005).
50. S. Q. Zheng et al., MotionCor2: anisotropic correction of beam-induced motion for improved cryo-electron microscopy. Nat. Methods. 14, 331-332 (2017).
51. D. N. Mastronarde, S. R. Held, Automated tilt sore alignment and tomographic reconstruction in IMOD. J. Struct. Biol. 197, 102-113 (2017).
52. A. Rohou, N. Grigorieff, CTFFIND4: Fast and accurate defocus estimation from electron micrographs. J. Struct. Biol. 192, 216-221 (2015).
53. C. A. Schneider, W. S. Rasband, K. W. Eliceiri, NIH Image to ImageJ: 25 years of image analysis. Nat. Methods. 9, 671-675 (2012).
54. Z. Diwu, C. S. Chen, C. L. Zhang, D. H. Klaubert, R. P. Haugland, in ABSTRACT OF PAPERS OF THE AMERICAN CHEMICAL SOCIETY (AMER CHEMICAL SOC 1155 16TH ST, NW, WASHINGTON, DC 20036 USA, 1999), vol. 218, pp. U27-U27.
55. L. L. C. Schrödinger, The PyMOL molecular graphics system, version 1.7. 6.6 (2015).
56. F. Busch et al., Localization of protein complex bound ligands by surface-induced dissociation high-resolution mass spectrometry. Anal. Chem. (2018), doi:10.1021/acs.analchem.8b03263.

1. A non-naturally occurring polypeptide or polypeptide oligomer, comprising a buried hydrogen bond network that comprises at least 1, 2, 3, 4, 5, 6, 7, 8, or 9 pH sensitive amino acids located (i) at an intra-chain interface between different structural elements in one polypeptide, or (ii) at an inter-chain interface between structural elements present in different chains of a polypeptide oligomer, wherein the polypeptide or polypeptide oligomer is stable above a given pH, and wherein the polypeptide or polypeptide oligomer undergoes a conformational transition when subjected to a pH at or below the given pH.

2.-6. (canceled)

7. The polypeptide or polypeptide oligomer of claim 1, wherein the buried hydrogen-bond network comprises one or more histidine-containing layers, wherein each histidine Nε and Nδ atoms are hydrogen-bonded across the one or more interfaces.

8.-9. (canceled)

10. A non-naturally occurring pH-responsive polypeptide, or polypeptide oligomer, comprising an oligomeric helical bundle comprising at least four alpha-helical subunits, wherein the oligomeric helical bundle comprises
one or more interfaces; and
one or more histidine-containing layers that participate in buried hydrogen bond networks, wherein each histidine Ne and NS atoms are hydrogen-bonded across the one or more interfaces;
wherein the polypeptide or polypeptide oligomer is stable above a given pH, and wherein oligomers (including but not limited to dimers or trimers) of the polypeptide undergo a conformational transition when subjected to a pH at or below the given pH.

11.-14. (canceled)

15. The polypeptide of claim 1, wherein the polypeptide is of the formula:
X1-X2-X3-X4-X5-X6-X7-X8-X9-X10-X11-X12-X13-X14-X15-X16-X17, wherein:
X1 and X17 are independently absent or comprise peptides;
X2, X4, X6, X8, X10, X12, X14, and X16 are each 1-2 amino acids that may be comprised of either hydrophobic residues or polar residues, forming a helical secondary structure, wherein at least 1, 2, 3, 4, 5, 6, 7, or all 8 of X2, X4, X6, X8, X10, X12, X14, and X16 include a histidine residue;
X3, X5, X7, X11, X13, and X15 are 5-6 residue variable amino acid linkers forming a helical secondary structure; and
X9 comprises a loop, including but not limited to a hairpin loop, of variable amino acids.

16. The polypeptide of claim 15, wherein 1, 2, 3, 4, 5, 6, or 7 of X2, X4, X6, X8, X10, X12, X14, and X16, when present are comprised of hydrophobic residues.

17. (canceled)

18. The polypeptide of claim 15, wherein each of X1 and X17 when present, are the same length, and/or wherein one or more of X1, X9 and X17 comprise a functional subunit.

19. (canceled)

20. The polypeptide of claim 1, wherein the polypeptide is of the formula:
X6-X7-X8-X9-X10-X11-X12, wherein; (I)
X6-X8 form a first helical secondary structure;
X10-X12 form a second helical structure;
X9 comprises a loop of variable amino acid length and sequence; and
wherein at least 1, 2, 3, 4, 5, or all 6 of X6, X7, X8, X10, X11, and X12 include a pH sensitive amino acid residue;
wherein the polypeptide or an oligomer comprising the polypeptide undergoes a conformational transition when subjected to a pH at or below the given pH;
X4-X5-X6-X7-X8-X9-X10-X11-X12-X13-X14, wherein: (II)
X4-X8 form a first helical secondary structure:
X10-X14 form a second helical structure:
X9 comprises a loop of variable amino acid length and sequence; and
wherein at least 1, 2, 3, 4, 5, 6, 7, 8, 9, or all 10 of X4, X5, X6, X7, X8, X10, X11, X12, X13, and X14 include a pH sensitive amino acid residue:
wherein the polypeptide or an oligomer comprising the polypeptide undergoes a conformational transition when subjected to a pH at or below the given pH; or
X2-X3-X4-X5-X6-X7-X8-X9-X10-X11-X12-X13-X14-X15-X16, wherein; (III)
X2-X8 form a first helical secondary structure;
X10-X16 form a second helical structure;
X9 comprises a loop of variable amino acid length and sequence; and
wherein at least 1, 2, 3, 4, 5, 6, 7, 8, 9, 10, 11, 12, 13, or all 14 of X2, X3, X4, X5, X6, X7, X8, X10, X11, X12, X13, X14, X15, and X16 include a pH sensitive amino acid residue;
wherein the polypeptide or an oligomer comprising the polypeptide undergoes a conformational transition when subjected to a pH at or below the given pH.

21.-22. (canceled)

23. The polypeptide of claim 20, wherein the pH sensitive amino acids are selected from the group consisting of histidine, aspartate, and glutamate residues, and/or wherein the polypeptide comprises at least 2, 3, 4, 5, 6, or more pH sensitive amino acids.

24.-25. (canceled)

26. The polypeptide of claim 20, wherein (a) 1, 2, 3, 4, 5, 6, 7, or all 8 of X2, X4, X6, X8, X10, X12, X14, and X16 (when present) are 1-2 amino acids that may be comprised of hydrophobic residues, polar residues or both, wherein at least 1, 2, 3, 4, 5, 6, 7, or all 8 of X2, X4, X6, X8, X10, X12, X14, and X16 (when present) include a pH sensitive amino acid, and (b) wherein 1, 2, 3, 4, 5, or all 6 of X3, X5, X7, X11, X13, and X15 (when present) are 5-6 residue variable amino acid linkers.

27. (canceled)

28. The polypeptide of claim 20, wherein X9 comprises a hairpin loop, or a flexible linker including but not limited to a flexible GS-based linker.

29.-30. (canceled)

31. The polypeptide of claim 1, comprising the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to the polypeptide of any one of SEQ ID NOS:1-40, 45-46, 60-66, 69-76, and 81-86.

32. The polypeptide of claim 31, wherein the polypeptide includes changes to the highlighted residues of SEQ ID NOS:1-36 in Tables 1-3 only to other polar amino acids, or wherein the polypeptide includes no changes to the highlighted residues of SEO ID NOS:1-36 in Tables 1-3.

33. (canceled)

34. The polypeptide of claim 31, wherein all amino acid substitutions relative to the amino acid sequence of SEQ ID NOS: 1-40, 45-46, 60-66, 69-76, and 81-86 are conservative amino acid substitutions.

35. A non-naturally occurring polypeptide, comprising the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to the amino acid sequence of one of SEQ ID NOS:1-77 and 81-86.

36. The polypeptide of claim 35, wherein the polypeptide includes changes to the highlighted residues of SEQ ID NOS:1-36 in Tables 1-3 only to other polar amino acids, or wherein the polypeptide includes no changes to the highlighted residues of SEO ID NOS:1-36 in Tables 1-3.

37.-38. (canceled)

39. An oligomeric polypeptide comprising two or more polypeptides of claim 10.

40. (canceled)

41. The oligomer of claim 39, comprising
(I) a heterodimer between polypeptides comprising the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to:
(a) the amino acid sequence of SEQ ID NO:81 and the amino acid sequence of SEQ ID NO:82;
(b) the amino acid sequence of SEQ ID NO:81 and the amino acid sequence of SEQ ID NO:84;
(c) the amino acid sequence of SEQ ID NO:83 and the amino acid sequence of SEQ ID NO:82;
(d) the amino acid sequence of SEQ ID NO:83 and the amino acid sequence of SEQ ID NO:84; or
(e) the amino acid sequence of SEQ ID NO:85 and the amino acid sequence of SEQ ID NO:86; or
(II) a homo-trimer of a polypeptide comprising the amino acid sequence at least 25%, 30%, 35%, 40%, 45%, 50%, 55%, 60%, 65%, 70%, 75%, 80%, 85%, 90%, 95%, 96%, 97%, 98%, 99%, or 100% identical to the amino acid sequence of one of SEO ID NOS: 1-26 or 33-36.

42.-43. (canceled)

44. A nucleic acid encoding the polypeptide of claim 1.

45. A recombinant expression vector comprising the nucleic acid of claim 44 operatively linked to a control sequence.

46. A recombinant host cell comprising the nucleic acid of claim 44.

47.-49. (canceled)


SEQUENCE LISTING (86 sequences)

SEQ ID NO: 1 - protein, 95 aa, Artificial Sequence
  Synthetic polypeptide
MGSHHHHHHGSGSENLYFQGSEYEIRKALEELKAATAELKRATASLRAITEELKKNPSEDALVEHNRAIVEHNAIIVEHNRIIAAVLELIVRAIK

SEQ ID NO: 2 - protein, 75 aa, Artificial Sequence
  Synthetic polypeptide
SEYEIRKALEELKAATAELKRATASLRAITEELKKNPSEDALVEHNRAIVEHNAIIVEHNRIIAAVLELIVRAIK

SEQ ID NO: 3 - protein, 95 aa, Artificial Sequence
  Synthetic polypeptide
MGSHHHHHHGSGSENLYFQGSEYEIRKALEELKAALAELKRATASLRAITEELKKNPSEDALVEHNRAIVEHNAIIVEVLRIIAAVLELIVRAIK

SEQ ID NO: 4 - protein, 75 aa, Artificial Sequence
  Synthetic polypeptide
SEYEIRKALEELKAALAELKRATASLRAITEELKKNPSEDALVEHNRAIVEHNAIIVEVLRIIAAVLELIVRAIK

SEQ ID NO: 5 - protein, 95 aa, Artificial Sequence
  Synthetic polypeptide
MGSHHHHHHGSGSENLYFQGSEYEIRKALEELKAALAELKRATASLRAILEELKKNPSEDAIVEAIRAIVEHNAIIVEVLRIIAAVLELIVRAIK

SEQ ID NO: 6 - protein, 75 aa, Artificial Sequence
  Synthetic polypeptide
SEYEIRKALEELKAALAELKRATASLRAILEELKKNPSEDAIVEAIRAIVEHNAIIVEVLRIIAAVLELIVRAIK

SEQ ID NO: 7 - protein, 95 aa, Artificial Sequence
  Synthetic polypeptide
MGSHHHHHHGSGSENLYFQGSEYEIRKALEELKASTAELKRATASLRAITEELKKNPSEDALVEHNRAIVEHNAIIVENNRIIAAVLELIVRAIK

SEQ ID NO: 8 - protein, 75 aa, Artificial Sequence
  Synthetic polypeptide
SEYEIRKALEELKASTAELKRATASLRAITEELKKNPSEDALVEHNRAIVEHNAIIVENNRIIAAVLELIVRAIK

SEQ ID NO: 9 - protein, 95 aa, Artificial Sequence
  Synthetic polypeptide
MGSHHHHHHGSGSENLYFQGSEYEIRKALEELKAATAELKRATASLRASTEELKKNPSEDALVENNRLIVEHNAIIVEHNRIIAAVLELIVRAIK

SEQ ID NO: 10 - protein, 75 aa, Artificial Sequence
  Synthetic polypeptide
SEYEIRKALEELKAATAELKRATASLRASTEELKKNPSEDALVENNRLIVEHNAIIVEHNRIIAAVLELIVRAIK

SEQ ID NO: 11 - protein, 95 aa, Artificial Sequence
  Synthetic polypeptide
MGSHHHHHHGSGSENLYFQGSEYEIRKALEELKASTAELKRATASLRASTEELKKNPSEDALVENNRLIVEHNAIIVENNRIIAAVLELIVRAIK

SEQ ID NO: 12 - protein, 75 aa, Artificial Sequence
  Synthetic polypeptide
SEYEIRKALEELKASTAELKRATASLRASTEELKKNPSEDALVENNRLIVEHNAIIVENNRIIAAVLELIVRAIK

SEQ ID NO: 13 - protein, 98 aa, Artificial Sequence
  Synthetic polypeptide
MGSSHHHHHHSSGLVPRGSHMGTLKEVLERLEEVLRRHREVAREHQRWAREHEQWVRDDPNSAKWIAESTRWILETTDAISRTADVLAEAIRVLAESD

SEQ ID NO: 14 - protein, 81 aa, Artificial Sequence
  Synthetic polypeptide
GSHMGTLKEVLERLEEVLRRHREVAREHQRWAREHEQWVRDDPNSAKWIAESTRWILETTDAISRTADVLAEAIRVLAESD

SEQ ID NO: 15 - protein, 95 aa, Artificial Sequence
  Synthetic polypeptide
MGSHHHHHHGSGSENLYFQGSEYEIRKALEELKAATAELKRATASLRAITEELKKNPSEDALVENNRAIVENNAIIVENNRIIAAVLELIVRAIK

SEQ ID NO: 16 - protein, 76 aa, Artificial Sequence
  Synthetic polypeptide
GSEYEIRKALEELKAATAELKRATASLRAITEELKKNPSEDALVENNRAIVENNAIIVENNRIIAAVLELIVRAIK

SEQ ID NO: 17 - protein, 95 aa, Artificial Sequence
  Synthetic polypeptide
MGSHHHHHHGSGSENLYFQGSEYEIRKALEELKAATAELKRATASLRAITEELKKNPSEDALVEHNRAIVEHNAIVVEHNRIIAAVLELIVRAIK

SEQ ID NO: 18 - protein, 76 aa, Artificial Sequence
  Synthetic polypeptide
GSEYEIRKALEELKAATAELKRATASLRAITEELKKNPSEDALVEHNRAIVEHNAIVVEHNRIIAAVLELIVRAIK

SEQ ID NO: 19 - protein, 95 aa, Artificial Sequence
  Synthetic polypeptide
MGSHHHHHHGSGSENLYFQGSEYEIRKALEELKAATAELKRATASLRAITEELKKNPSEDALVEHNRAIVEHNMIIVEHNRIIAAVLELIVRAIK

SEQ ID NO: 20 - protein, 76 aa, Artificial Sequence
  Synthetic polypeptide
GSEYEIRKALEELKAATAELKRATASLRAITEELKKNPSEDALVEHNRAIVEHNMIIVEHNRIIAAVLELIVRAIK

SEQ ID NO: 21 - protein, 95 aa, Artificial Sequence
  Synthetic polypeptide
MGSHHHHHHGSGSENLYFQGSEYEIRKALEELKAATAELKRATASLRAITEELKKNPSEDALVEHNRAIVEHNAIIVEHNRIIAAVLELNVRAIK

SEQ ID NO: 22 - protein, 76 aa, Artificial Sequence
  Synthetic polypeptide
GSEYEIRKALEELKAATAELKRATASLRAITEELKKNPSEDALVEHNRAIVEHNAIIVEHNRIIAAVLELNVRAIK

SEQ ID NO: 23 - protein, 83 aa, Artificial Sequence
  Synthetic polypeptide
MGSHHHHHHGSGSENLYFQGSEALYELEKALRELKKATAALERATAELKKNPSEDALVEHNRLIAAHNKIIAEVLRIIAKVLK

SEQ ID NO: 24 - protein, 64 aa, Artificial Sequence
  Synthetic polypeptide
GSEALYELEKALRELKKATAALERATAELKKNPSEDALVEHNRLIAAHNKIIAEVLRIIAKVLK

SEQ ID NO: 25 - protein, 83 aa, Artificial Sequence
  Synthetic polypeptide
MGSHHHHHHGSGSENLYFQGSEALYELEKATRELKKATDELERATEELEKNPSEDALVEHNRLIAEHNKIIAEHNRIIAKVLK

SEQ ID NO: 26 - protein, 64 aa, Artificial Sequence
  Synthetic polypeptide
GSEALYELEKATRELKKATDELERATEELEKNPSEDALVEHNRLIAEHNKIIAEHNRIIAKVLK

SEQ ID NO: 27 - protein, 165 aa, Artificial Sequence
  Synthetic polypeptide
MDEEDHLKKLKTHLEKLERHLKLLEDHAKKLEDILKERPEDSAVKESIDELRRSIELVRESIEIFRQSVEEEEGSSHHHHHHSSGENLYFQGDVKELTKILDTLTKILETATKVIKDATKLLEEHRKSDKPDPRLIETHKKLVEEHETLVRQHKELAEEHLKRTR

SEQ ID NO: 28 - protein, 147 aa, Artificial Sequence
  Synthetic polypeptide
MDEEDHLKKLKTHLEKLERHLKLLEDHAKKLEDILKERPEDSAVKESIDELRRSIELVRESIEIFRQSVEEEEGDVKELTKILDTLTKILETATKVIKDATKLLEEHRKSDKPDPRLIETHKKLVEEHETLVRQHKELAEEHLKRTR

SEQ ID NO: 29 - protein, 165 aa, Artificial Sequence
  Synthetic polypeptide
MDEEDHLKKLKTHLEKLERHLKLAEDHAKKLEDILKERPEDSAVKESIDELRRSIELVRESIEIFRQSVEEEEGSSHHHHHHSSGENLYFQGDVKELTKILDTLTKILETATKVIKDATKLLEEHRKSDKPDPRLIETHKKLVEEHETLARQHKELAEEHLKRTR

SEQ ID NO: 30 - protein, 147 aa, Artificial Sequence
  Synthetic polypeptide
MDEEDHLKKLKTHLEKLERHLKLAEDHAKKLEDILKERPEDSAVKESIDELRRSIELVRESIEIFRQSVEEEEGDVKELTKILDTLTKILETATKVIKDATKLLEEHRKSDKPDPRLIETHKKLVEEHETLARQHKELAEEHLKRTR

SEQ ID NO: 31 - protein, 172 aa, Artificial Sequence
  Synthetic polypeptide
MTKEDILERQRKIIERAQEIHRRQQEILKEQEKIIRKPGSSEEAMKRSLKLIEESLRLLKELLELSEESAQLLYEQRGSSHHHHHHSSGENLYFQGTEKRLLEEAERAHREQKEIIKKAQELHKELTKIHQQSGSSEEAKKRALKISQEIRELSKRSLELLREILYLSQEQK

SEQ ID NO: 32 - protein, 154 aa, Artificial Sequence
  Synthetic polypeptide
MTKEDILERQRKIIERAQEIHRRQQEILKEQEKIIRKPGSSEEAMKRSLKLIEESLRLLKELLELSEESAQLLYEQRGTEKRLLEEAERAHREQKEIIKKAQELHKELTKIHQQSGSSEEAKKRALKISQEIRELSKRSLELLREILYLSQEQK

SEQ ID NO: 33 - protein, 99 aa, Artificial Sequence
  Synthetic polypeptide
MGSHHHHHHGSGSENLYFQGSEYEIRKALEELKAATAELKRATASLRAITEELKKGGSGSGSEDALVEHNRAIVEHNAIIVEHNRIIAAVLELIVRAIK

SEQ ID NO: 34 - protein, 80 aa, Artificial Sequence
  Synthetic polypeptide
GSEYEIRKALEELKAATAELKRATASLRAITEELKKGGSGSGSEDALVEHNRAIVEHNAIIVEHNRIIAAVLELIVRAIK

SEQ ID NO: 35 - protein, 99 aa, Artificial Sequence
  Synthetic polypeptide
MGSHHHHHHGSGSENLYFQGSEYEIRKALEELKASTAELKRATASLRAITEELKKGGSGSGSEDALVEHNRAIVEHNAIIVENNRIIAAVLELIVRAIK

SEQ ID NO: 36 - protein, 80 aa, Artificial Sequence
  Synthetic polypeptide
GSEYEIRKALEELKASTAELKRATASLRAITEELKKGGSGSGSEDALVEHNRAIVEHNAIIVENNRIIAAVLELIVRAIK

SEQ ID NO: 37 - protein, 248 aa, Artificial Sequence
  Synthetic polypeptide
GSEEEIKRLLEELRKSSEELRRITKELDDLSKELRVGGSGSGSEMLVEHNKLISEHNRIIVENNRIIVEILEAIARVGGSGSGSVEVERILDELRKSSEELDRVTKELKKLTEELDVGGSENLYFQGSGSVEALVRHNVLITRHNDIIVKNNDIINKILKLIAEAVGGSGSGSELERILRELEESTKELRKATEELRRLSEELKVGGSGSGSVEALVRHNEAIVEHNKIIVKNNDIIVKILELITERI

SEQ ID NO: 38 - protein, 248 aa, Artificial Sequence
  Synthetic polypeptide
GSEEEIKRLLEELRKSSEELRRITKELDDLSKELRVGGSGSGSEMLVEHNKLISEHNRIIVENNRIIVEILEAIARVGGSGSGSVEVERILDELRKSSEELDRVTKELKKLTEELDVGGSGSGSVEALVRHNVLITRHNDIIVKNNDIINKILKLIGEAVGGSENLYFQGSGSEFERWLRQLEESTKELRKFTEELRRFSEELKVGGSGSGSVEALVRHNEAIVEHNKAIVKNNDIIVKILELVTERI

SEQ ID NO: 39 - protein, 278 aa, Artificial Sequence
  Synthetic polypeptide
GSEEEIKRLLEELRKSSEELRRITKELDDLSKELRVGGSGSGSLVPRGSGSGSGSHALVEHNKLISEHNRIVVENNRIIVEILEAIARVGGSGSGSVEVERILDELRKSSEELDRVTKELKKLTEELDVGGSGSGSLVPRGSGSGSGSVEALVRHNVLITRHNDIVVKNNDIINKILKLIAEAVGGSGSGSELERILRELEESTKELRKATEELRRLSEELKVGGSGSGSLVPRGSGSGSGSHEALVRHNEAIVEHNKIVVKNNDIIVKILELITERI

SEQ ID NO: 40 - protein, 278 aa, Artificial Sequence
  Synthetic polypeptide
GSEEEIKRLLEELRKALEELRRITKELDDLSKELRVGGSGSGSLVPRGSGSGSGSHALVEHNKLISEHNRIVVEVLRIIAEILEAIARVGGSGSGSVEVERILDELRKALEELDRVTKELKKLTEELDVGGSGSGSLVPRGSGSGSGSVEALVRHNVLITRHNDIVVKVLDIIAKILKLIAEAVGGSGSGSELERILRELEEALKELRKATEELRRLSEELKVGGSGSGSLVPRGSGSGSGSHEALVRHNEAIVEHNKIVVKVLDIIAKILELITERI

SEQ ID NO: 41 - protein, 228 aa, Artificial Sequence
  Synthetic polypeptide
MGSSHHHHHHSSGLVPRGSHMGSDELKYELEKSTRELQKSTDELEKSTEELERNPSKDVLVENNELIVRNNKIIVKNNIIIVRTEKKGSGGSGDELKEELEKSTRELDKSTKKLERSTEELKRNPSKDALVENNKLIVENNTIIVRNNDIIVRTRKKGSGGSGDELKEELEKSTRELKKSTKELQKSTEELERNPSKDALVKNNKLIADNNRIIVRNNTIIVRDIKAS

SEQ ID NO: 42 - protein, 209 aa, Artificial Sequence
  Synthetic polypeptide
HMGSDELKYELEKSTRELQKSTDELEKSTEELERNPSKDVLVENNELIVRNNKIIVKNNIIIVRTEKKGSGGSGDELKEELEKSTRELDKSTKKLERSTEELKRNPSKDALVENNKLIVENNTIIVRNNDIIVRTRKKGSGGSGDELKEELEKSTRELKKSTKELQKSTEELERNPSKDALVKNNKLIADNNRIIVRNNTIIVRDIKAS

SEQ ID NO: 43 - protein, 261 aa, Artificial Sequence
  Synthetic polypeptide
MGSSHHHHHHSSGLVPRGSHMGSDDEDIDRVLEELRRSTEELDRSTKDLERSTQELRRNPSVDALVKNNNAIVRNNEIIVENNRIILEVLELLLRSIKGSGGSGDREEIKKVLDELRESTERLERSTEELRRSTEELKKNPAVEVLVRNNTIIVKNNKIIVDNNRIIVRVLELLEKTIKGSGGSGDKYEIRKVLKELKDSTEELRNSTKNLTDSTEELKRNPSVEILVKNNILIVENNKIIVENNRIIVDVLELIRKAIAS

SEQ ID NO: 44 - protein, 242 aa, Artificial Sequence
  Synthetic polypeptide
HMGSDDEDIDRVLEELRRSTEELDRSTKDLERSTQELRRNPSVDALVKNNNAIVRNNEIIVENNRIILEVLELLLRSIKGSGGSGDREEIKKVLDELRESTERLERSTEELRRSTEELKKNPAVEVLVRNNTIIVKNNKIIVDNNRIIVRVLELLEKTIKGSGGSGDKYEIRKVLKELKDSTEELRNSTKNLTDSTEELKRNPSVEILVKNNILIVENNKIIVENNRIIVDVLELIRKAIAS

SEQ ID NO: 45 - protein, 261 aa, Artificial Sequence
  Synthetic polypeptide
MGSSHHHHHHSSGLVPRGSHMGSDDEDIDRVLEELRRITEELDRITKDLERLTQELRRNPSVDALVKHNNAIVRHNEIIVEHNRIILEVLELLLRSIKGSGGSGDREEIKKVLDELREATERLERATEELRRLTEELKKNPAVEVLVRHNTIIVKHNKIIVDHNRIIVRVLELLEKTIKGSGGSGDKYEIRKVLKELKDITEELRNMTKNLTDLTEELKRNPSVEILVKHNILIVEHNKIIVEHNRIIVDVLELIRKAIAS

SEQ ID NO: 46 - protein, 242 aa, Artificial Sequence
  Synthetic polypeptide
HMGSDDEDIDRVLEELRRITEELDRITKDLERLTQELRRNPSVDALVKHNNAIVRHNEIIVEHNRIILEVLELLLRSIKGSGGSGDREEIKKVLDELREATERLERATEELRRLTEELKKNPAVEVLVRHNTIIVKHNKIIVDHNRIIVRVLELLEKTIKGSGGSGDKYEIRKVLKELKDITEELRNMTKNLTDLTEELKRNPSVEILVKHNILIVEHNKIIVEHNRIIVDVLELIRKAIAS

SEQ ID NO: 47 - protein, 489 aa, Artificial Sequence
  Synthetic polypeptide
MGSSHHHHHHSSGLVPRGSHMSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTGGSGGSENLYFQGASGGSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARY

SEQ ID NO: 48 - protein, 470 aa, Artificial Sequence
  Synthetic polypeptide
HMSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTGGSGGSENLYFQGASGGSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARY

SEQ ID NO: 49 - protein, 685 aa, Artificial Sequence
  Synthetic polypeptide
MGSSHHHHHHSSGLVPRGSHMSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTGGSGGSDELKYELEKSTRELQKSTDELEKSTEELERNPSKDVLVENNELIVRNNKIIVKNNIIIVRTEKKGSGGSGDELKEELEKSTRELDKSTKKLERSTEELKRNPSKDALVENNKLIVENNTIIVRNNDIIVRTRKKGSGGSGDELKEELEKSTRELKKSTKELQKSTEELERNPSKDALVKNNKLIADNNRIIVRNNTIIVRDIKASGGSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARY

SEQ ID NO: 50 - protein, 666 aa, Artificial Sequence
  Synthetic polypeptide
HMSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTGGSGGSDELKYELEKSTRELQKSTDELEKSTEELERNPSKDVLVENNELIVRNNKIIVKNNIIIVRTEKKGSGGSGDELKEELEKSTRELDKSTKKLERSTEELKRNPSKDALVENNKLIVENNTIIVRNNDIIVRTRKKGSGGSGDELKEELEKSTRELKKSTKELQKSTEELERNPSKDALVKNNKLIADNNRIIVRNNTIIVRDIKASGGSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARY

SEQ ID NO: 51 - protein, 685 aa, Artificial Sequence; identical to SEQ ID NO: 49
  Synthetic polypeptide

SEQ ID NO: 52 - protein, 666 aa, Artificial Sequence; identical to SEQ ID NO: 50
  Synthetic polypeptide

SEQ ID NO: 53 - protein, 268 aa, Artificial Sequence
  Synthetic polypeptide
MGSSHHHHHHSSGENLYFQGSGSGGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEY

SEQ ID NO: 54 - protein, 251 aa, Artificial Sequence
  Synthetic polypeptide
FQGSGSGGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEY

SEQ ID NO: 55 - protein, 502 aa, Artificial Sequence
  Synthetic polypeptide
MGSSHHHHHHSSGENLYFQGSGSGSTKYELRRALEELEKALRELKKSLDELERSLEELEKNPSEDALVENNRLNVENNKIIVEVLRIIAEVLKINAKSDGSGSGSTKYELRRALEELEKALRELKKSLDELERSLEELEKNPSEDALVENNRLNVENNKIIVEVLRIIAEVLKINAKSDGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGGSTKYELRRALEELEKALRELKKSLDELERSLEELEKNPSEDALVENNRLNVENNKIIVEVLRIIAEVLKINAKSD

SEQ ID NO: 56 - protein, 485 aa, Artificial Sequence
  Synthetic polypeptide
FQGSGSGSTKYELRRALEELEKALRELKKSLDELERSLEELEKNPSEDALVENNRLNVENNKIIVEVLRIIAEVLKINAKSDGSGSGSTKYELRRALEELEKALRELKKSLDELERSLEELEKNPSEDALVENNRLNVENNKIIVEVLRIIAEVLKINAKSDGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGGSTKYELRRALEELEKALRELKKSLDELERSLEELEKNPSEDALVENNRLNVENNKIIVEVLRIIAEVLKINAKSD

SEQ ID NO: 57 - protein, 724 aa, Artificial Sequence
  Synthetic polypeptide
FQGSGSGSTKYELRRALEELEKALRELKKSLDELERSLEELEKNPSEDALVENNRLNVENNKIIVEVLRIIAEVLKINAKSDGSGSGSTKYELRRALEELEKALRELKKSLDELERSLEELEKNPSEDALVENNRLNVENNKIIVEVLRIIAEVLKINAKSDGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGGSTKYELRRALEELEKALRELKKSLDELERSLEELEKNPSEDALVENNRLNVENNKIIVEVLRIIAEVLKINAKSDMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 58 - protein, 742 aa, Artificial Sequence
  Synthetic polypeptide
MGSSHHHHHHSSGENLYFQGSGSGSTKYELRRALEELEKALRELKKSLDELERSLEELEKNPSEDALVENNRLNVENNKIIVEVLRIIAEVLKINAKSDGSGSGSTKYELRRALEELEKALRELKKSLDELERSLEELEKNPSEDALVENNRLNVENNKIIVEVLRIIAEVLKINAKSDGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSTKYELRRALEELEKALRELKKSLDELERSLEELEKNPSEDALVENNRLNVENNKIIVEVLRIIAEVLKINAKSDMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 59 - protein, 725 aa, Artificial Sequence
  Synthetic polypeptide
FQGSGSGSTKYELRRALEELEKALRELKKSLDELERSLEELEKNPSEDALVENNRLNVENNKIIVEVLRIIAEVLKINAKSDGSGSGSTKYELRRALEELEKALRELKKSLDELERSLEELEKNPSEDALVENNRLNVENNKIIVEVLRIIAEVLKINAKSDGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSTKYELRRALEELEKALRELKKSLDELERSLEELEKNPSEDALVENNRLNVENNKIIVEVLRIIAEVLKINAKSDMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 60 - protein, 466 aa, Artificial Sequence
  Synthetic polypeptide
MGSSHHHHHHSSGENLYFQGSGSGSEALYELEKATRELKKATDELERATEELEKNPSEDALVEHNRLIAEHNKIIAEHNRIIAKVLKGSGSGSEALYELEKATRELKKATDELERATEELEKNPSEDALVEHNRLIAEHNKIIAEHNRIIAKVLKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGGSEALYELEKATRELKKATDELERATEELEKNPSEDALVEHNRLIAEHNKIIAEHNRIIAKVLK

SEQ ID NO: 61 - protein, 449 aa, Artificial Sequence
  Synthetic polypeptide
FQGSGSGSEALYELEKATRELKKATDELERATEELEKNPSEDALVEHNRLIAEHNKIIAEHNRIIAKVLKGSGSGSEALYELEKATRELKKATDELERATEELEKNPSEDALVEHNRLIAEHNKIIAEHNRIIAKVLKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGGSEALYELEKATRELKKATDELERATEELEKNPSEDALVEHNRLIAEHNKIIAEHNRIIAKVLK

SEQ ID NO: 62 - protein, 467 aa, Artificial Sequence
  Synthetic polypeptide
MGSSHHHHHHSSGENLYFQGSGSGSEALYELEKATRELKKATDELERATEELEKNPSEDALVEHNRLIAEHNKIIAEHNRIIAKVLKGSGSGSEALYELEKATRELKKATDELERATEELEKNPSEDALVEHNRLIAEHNKIIAEHNRIIAKVLKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSEALYELEKATRELKKATDELERATEELEKNPSEDALVEHNRLIAEHNKIIAEHNRIIAKVLK

SEQ ID NO: 63 - protein, 450 aa, Artificial Sequence
  Synthetic polypeptide
FQGSGSGSEALYELEKATRELKKATDELERATEELEKNPSEDALVEHNRLIAEHNKIIAEHNRIIAKVLKGSGSGSEALYELEKATRELKKATDELERATEELEKNPSEDALVEHNRLIAEHNKIIAEHNRIIAKVLKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSEALYELEKATRELKKATDELERATEELEKNPSEDALVEHNRLIAEHNKIIAEHNRIIAKVLK

SEQ ID NO: 64 - protein, 467 aa, Artificial Sequence
  Synthetic polypeptide
MGSSHHHHHHSSGENLYFQGSGSGSEALYELEKATRELKKATDELERATEELEKNPSEDALVEHNRLIAEHNKIVAEHNRIIAKVLKGSGSGSEALYELEKATRELKKATDELERATEELEKNPSEDALVEHNRLIAEHNKIVAEHNRIIAKVLKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSEALYELEKATRELKKATDELERATEELEKNPSEDALVEHNRLIAEHNKIVAEHNRIIAKVLK

SEQ ID NO: 65 - protein, 450 aa, Artificial Sequence
  Synthetic polypeptide
FQGSGSGSEALYELEKATRELKKATDELERATEELEKNPSEDALVEHNRLIAEHNKIVAEHNRIIAKVLKGSGSGSEALYELEKATRELKKATDELERATEELEKNPSEDALVEHNRLIAEHNKIVAEHNRIIAKVLKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSEALYELEKATRELKKATDELERATEELEKNPSEDALVEHNRLIAEHNKIVAEHNRIIAKVLK

SEQ ID NO: 66 - protein, 686 aa, Artificial Sequence
  Synthetic polypeptide
MGSGSEALYELEKATRELKKATDELERATEELEKNPSEDALVEHNRLIAEHNKIIAEHNRIIAKVLKGSGSGSEALYELEKATRELKKATDELERATEELEKNPSEDALVEHNRLIAEHNKIIAEHNRIIAKVLKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSEALYELEKATRELKKATDELERATEELEKNPSEDALVEHNRLIAEHNKIIAEHNRIIAKVLKMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 67 - protein, 511 aa, Artificial Sequence
  Synthetic polypeptide
MGSSHHHHHHSSGENLYFQGSGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSGMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 68 - protein, 494 aa, Artificial Sequence
  Synthetic polypeptide
FQGSGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSGMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 69 - protein, 739 aa, Artificial Sequence
  Synthetic polypeptide
MGSSHHHHHHSSGENLYFQGSGSGDDEDIDRVLEELRRITEELDRITKDLERLTQELRRNPSVDALVKHNNAIVRHNEIIVEHNRIILEVLELLLRSIGSGSGDREEIKKVLDELREATERLERATEELRRLTEELKKNPAVEVLVRHNTIIVKHNKIIVDHNRIIVRVLELLEKTIGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGDKYEIRKVLKELKDITEELRNMTKNLTDLTEELKRNPSVEILVKHNILIVEHNKIIVEHNRIIVDVLELIRKAIMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 70 - protein, 722 aa, Artificial Sequence
  Synthetic polypeptide
FQGSGSGDDEDIDRVLEELRRITEELDRITKDLERLTQELRRNPSVDALVKHNNAIVRHNEIIVEHNRIILEVLELLLRSIGSGSGDREEIKKVLDELREATERLERATEELRRLTEELKKNPAVEVLVRHNTIIVKHNKIIVDHNRIIVRVLELLEKTIGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGDKYEIRKVLKELKDITEELRNMTKNLTDLTEELKRNPSVEILVKHNILIVEHNKIIVEHNRIIVDVLELIRKAIMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 71 - protein, 668 aa, Artificial Sequence
  Synthetic polypeptide
MGSSHHHHHHSSGENLYFQGSGSGSDKEYKLDRILRRLDELIKQLSRILEEIERLVDELEREPLDDKEVQDVIERIVELIDEHLELLKEYIKLLEEYIKTTKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSPSKEYQEKSAERQKELLHEYEKLVRHLRELVEKLQRRELDKEEVLRRLVEILERLKDLHKKIEDAHRKNEEAHKENKMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 72 - protein, 651 aa, Artificial Sequence
  Synthetic polypeptide
FQGSGSGSDKEYKLDRILRRLDELIKQLSRILEEIERLVDELEREPLDDKEVQDVIERIVELIDEHLELLKEYIKLLEEYIKTTKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSPSKEYQEKSAERQKELLHEYEKLVRHLRELVEKLQRRELDKEEVLRRLVEILERLKDLHKKIEDAHRKNEEAHKENKMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 73 - protein, 668 aa, Artificial Sequence
  Synthetic polypeptide
MGSSHHHHHHSSGENLYFQGSGSGSPSKEYQEKSAERQKELLHEYEKLVRHLRELVEKLQRRELDKEEVLRRLVEILERLKDLHKKIEDAHRKNEEAHKENKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSDKEYKLDRILRRLDELIKQLSRILEEIERLVDELEREPLDDKEVQDVIERIVELIDEHLELLKEYIKLLEEYIKTTKMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 74 - protein, 651 aa, Artificial Sequence
  Synthetic polypeptide
FQGSGSGSPSKEYQEKSAERQKELLHEYEKLVRHLRELVEKLQRRELDKEEVLRRLVEILERLKDLHKKIEDAHRKNEEAHKENKGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGSGSDKEYKLDRILRRLDELIKQLSRILEEIERLVDELEREPLDDKEVQDVIERIVELIDEHLELLKEYIKLLEEYIKTTKMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 75 - protein, 95 aa, Artificial Sequence
  Synthetic polypeptide
MGSHHHHHHGSGSENLYFQGSEYEIRKALEELKASTAELKRSTASLRASTEELKKNPSEDALVENNRLIVENNAIIVENNRIIAAVLELIVRAIK

SEQ ID NO: 76 - protein, 76 aa, Artificial Sequence
  Synthetic polypeptide
GSEYEIRKALEELKASTAELKRSTASLRASTEELKKNPSEDALVENNRLIVENNAIIVENNRIIAAVLELIVRAIK

SEQ ID NO: 77 - protein, 741 aa, Artificial Sequence
  Synthetic polypeptide
MGSSHHHHHHSSGENLYFQGSGSGSTKYELRRALEELEKALRELKKSLDELERSLEELEKNPSEDALVENNRLNVENNKIIVEVLRIIAEVLKINAKSDGSGSGSTKYELRRALEELEKALRELKKSLDELERSLEELEKNPSEDALVENNRLNVENNKIIVEVLRIIAEVLKINAKSDGSGIHGNVYITADKQKNGIKANFGLNSNVEDGSVQLADHYQQNTPIGDGPVLLPDNHYLSTQSALSKDPNEKRDHMVLLEFVTAAGITLGMDELYKGGTGGSMVSKGEELFTGVVPILVELDGDVNGHKFSVRGEGEGDATNGKLTLKFISTTGKLPVPWPTLVTTLSWGVQSFARYPDHMKQHDFFKSAMPEGYVQERTIFFKDDGTYKTRAEVKFEGDTLVNRIELKGIDFKEDGNILGHKLEYGGSTKYELRRALEELEKALRELKKSLDELERSLEELEKNPSEDALVENNRLNVENNKIIVEVLRIIAEVLKINAKSDMVSKGEELFTGVVPILVELDGDVNGHKFSVSGEGEGDATYGKLTLKFISTTGKLPVPWPTLVTTLTYGVQMFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 78 - protein, 17 aa, Artificial Sequence
  pRO-2
HNRIIAAVLELIVRAIK

SEQ ID NO: 79 - protein, 17 aa, Artificial Sequence
  pRO-3
HNKIIAEVLRIIAKVLK

SEQ ID NO: 80 - protein, 17 aa, Artificial Sequence
  pRO-3.1
HNKIIAEHNRIIAKVLK

SEQ ID NO: 81 - protein, 73 aa, Artificial Sequence
  Synthetic polpypeptide
MDEEDHLKKLKTHLEKLERHLKLLEDHAKKLEDILKERPEDSAVKESIDELRRSIELVRESIEIFRQSVEEEE

SEQ ID NO: 82 - protein, 92 aa, Artificial Sequence
  Synthetic polpypeptide
  MISC_FEATURE (1)..(18): Optional residues
GSSHHHHHHSSGENLYFQGDVKELTKILDTLTKILETATKVIKDATKLLEEHRKSDKPDPRLIETHKKLVEEHETLVRQHKELAEEHLKRTR

SEQ ID NO: 83 - protein, 73 aa, Artificial Sequence
  Synthetic polpypeptide
MDEEDHLKKLKTHLEKLERHLKLAEDHAKKLEDILKERPEDSAVKESIDELRRSIELVRESIEIFRQSVEEEE

SEQ ID NO: 84 - protein, 92 aa, Artificial Sequence
  Synthetic polpypeptide
  MISC_FEATURE (1)..(18): Optional residues
GSSHHHHHHSSGENLYFQGDVKELTKILDTLTKILETATKVIKDATKLLEEHRKSDKPDPRLIETHKKLVEEHETLARQHKELAEEHLKRTR

SEQ ID NO: 85 - protein, 77 aa, Artificial Sequence
  Synthetic polpypeptide
MTKEDILERQRKIIERAQEIHRRQQEILKEQEKIIRKPGSSEEAMKRSLKLIEESLRLLKELLELSEESAQLLYEQR

SEQ ID NO: 86 - protein, 95 aa, Artificial Sequence
  Synthetic polpypeptide
  MISC_FEATURE (1)..(18): Optional residues
GSSHHHHHHSSGENLYFQGTEKRLLEEAERAHREQKEIIKKAQELHKELTKIHQQSGSSEEAKKRALKISQEIRELSKRSLELLREILYLSQEQK